(19) United States
(12) Patent Application Publication
(10) Pub. No.: US 2022/0175667 A1
(43) Pub. Date: Jun. 9, 2022

(54) Extracellular Matrices, Uses Thereof, and Methods of Making Extracellular Matrices

(71) Applicant: Johns Hopkins University, Baltimore, MD (US)

(72) Inventors: Emily English, Baltimore, MD (US); Matthew DAVENPORT, Baltimore, MD (US); Janty SHOGA, Baltimore, MD (US)

(21) Appl. No.: 17/597,072

(22) PCT Filed: Jun. 26, 2020
(86) PCT No.: PCT/US2020/039864

§ 371 Date: Dec. 23, 2021

Related U.S. Application Data
(60) Provisional application No. 62/867,069, filed on Jun. 26, 2019.

(51) Int. Cl. A61K 9/00; A61K 47/32; A61K 47/36; A61K 35/33; A61K 35/44; A61P 17/02

(52) U.S. Cl. CPC A61K 9/0024; A61K 47/32; A61P 17/02; A61K 35/33; A61K 35/44; A61K 47/36

(57) ABSTRACT

The invention is directed to extracellular matrix replacement (EMR)-drug conjugates, EMR-fluorescent label conjugates, EMR-cell compositions; to methods of making the EMR-drug conjugates, EMR-fluorescent label conjugates, or EMR-cell compositions; to pharmaceutical compositions comprising the EMR-drug conjugates, EMR-fluorescent label conjugates, or EMR-cell combinations; and to methods of treating wounds using the EMR-drug conjugates, EMR-fluorescent label conjugates, or EMR-cell compositions. The invention is also directed to cure-in-place (CIP) EMRs, to methods of making the CIP EMRs, to pharmaceutical compositions comprising the CIP EMRs, and methods of treating wounds using the CIP EMRs.

### BACKGROUND

In developed countries, aging populations and escalating rates of diabetes and obesity have led to the prevalence of chronic wounds. Chronic wounds are estimated to affect more than 6.5 million patients in the United States alone, and the annual healthcare burden associated with their treatment is estimated to be more than $25 billion. Chronic wounds have irregular boundaries and vary greatly in size, shape, and depth. Although the market is flooded with products for treating these chronic wounds, current products are expensive and show limited clinical effectiveness. Further, current products offer little capability to fit the precise shape and size of a wound bed.

Normal wound healing requires interactions of numerous cell types, soluble mediators, and the extracellular milieu to proceed through the wound healing cascade: inflammation, re-epithelialization, angiogenesis, tissue formation, wound contraction, and tissue remodeling. Chronic wounds remain stalled in the inflammatory stage.

One type of chronic wound is the diabetic ulcer, which is painful for diabetic patients and requires expensive long-term treatment. Diabetic ulcers often remain unhealed for over eight weeks and frequently reoccur. In the worst circumstances, diabetic ulcers lead to limb amputations. For example, diabetic foot ulcers precede 84% of all diabetes-related lower-leg amputations. In addition, these diabetic ulcers often recur, generally because they are unable to progress through the stages of normal wound healing. Diabetic patients suffer from macrovascular disease and microcirculatory deficiencies that reduce capillary size, impair endothelial function, and cause abnormal blood flow. These factors, combined with neuropathy, may render diabetic patients susceptible to pressure forces in their numbed feet, which may lead to foot ulcers and eventually to non-healing chronic wounds.

A significant and common problem associated with chronic wounds is infection, which contributes to their chronicity. Current treatment for non-healing chronic diabetic foot ulcers includes debridement followed by application of bioactive dressings and skin substitutes. These bioactive dressings and skin substitutes are expensive, require specialized training for application, have a short shelf life, and are unable to be further processed to incorporate active pharmaceutical ingredients. Because chronic wounds have been difficult to heal and have a high rate of recurrence, the effective treatment of chronic wounds remains an unmet challenge.

### SUMMARY OF THE INVENTION

The invention described herein is directed to extracellular matrix replacement (EMR)-comprising a pharmaceutically active compound, e.g., a small molecule/drug. In some embodiments the drug is conjugated to the EMR, (“EMR-drug conjugates”) and in some embodiments the drug is trapped within the spaces/pores of the EMR (“interstitial EMR drug compositions”). The invention described herein is also directed to methods of making the EMRs comprising the drugs, to pharmaceutical compositions comprising the EMRs comprising the drugs, and to methods of treating wounds using the EMRs comprising the drugs.

The invention described herein is also directed to fluorescently labeled EMRs, to methods of making the fluorescently labeled EMRs, to pharmaceutical compositions comprising the fluorescently labeled EMRs, and to methods of treating wounds using the fluorescently labeled EMRs.

The invention described herein is also directed to cure-in-place EMRs, to methods of making the cure-in-place EMRs, to pharmaceutical compositions comprising the cure-in-place EMRs, and methods of treating wounds using the cure-in-place EMRs. The cure-in-place EMRs of this invention effectively conform to the size, shape, and depth of a wound bed thereby filling the wound bed and further enhancing wound healing.

The invention described herein is also directed to a composition comprising an EMR and cells (“EMR-cell composition”). The invention described herein is also directed to methods of making the EMR-cell compositions, to pharmaceutical compositions comprising the EMR-cell compositions, and to methods of treating wounds using the EMR-cell compositions.

### BRIEF DESCRIPTION OF THE FIGURES

FIG. 1. Scheme for incorporating polyethylene(glycol)diacrylate into dextramate and for incorporating polyethylene(glycol)diacrylate and acrylate-polyethylene(glycol)-succinimidyl valeric acid into dextramate.

FIG. 2A. Scheme for reacting 5-fluoresceinamine with an EMR.

FIG. 2B. Scheme for reacting 5-fluoresceinamine with an EMR.

FIG. 3A. Scheme for reacting valsartan with polyethylene(glycol)acrylate.

FIG. 3B. Scheme for reacting a small molecule comprising a carboxylic acid group with polyethylene(glycol)acrylate.

FIG. 4A. Scheme for incorporating polyethylene(glycol)diacrylate and a valsartan-substituted polyethylene(glycol)acrylate into dextramate.

FIG. 4B. Scheme for incorporating polyethylene(glycol)diacrylate and a small molecule-substituted polyethylene(glycol)acrylate into dextramate.

FIG. 5A. Fluorescein-labeled gel imaged immediately after labeling.

FIG. 5B. Fluorescein-labeled gel imaged after labeling and after washing.

FIG. 6: Optical Power Output at Various Wavelengths by Various Curing Light Sources over 30 min at a Distance of 5 cm—Scheme for showing various frequency lightwaves used to cure cell cultures.

FIG. 7A: Proliferation of hMSCs and NuFFs after light exposure to 415 nm wavelength LED, 365 nm wavelength LED, and UV Lamps.

FIG. 7B: Live/dead stain of NuFFs exposed to light from UV lamps, a 365-nm LED, and a 415-nm LED light at 1 and 2 days post-seeding. Green staining indicates live cells; red staining indicates dead cells.

FIG. 8A: Depicts the viability of a live fraction of hMSCs in EMR-cell compositions over 0 to 10 days. Data points of EMR-cell compositions that were prepared by curing using an Irgacure catalyst are depicted as circles. Data points of EMR-cell compositions that were prepared by curing using a LAP catalyst are depicted as squares.

FIG. 8B: Depicts the proliferation of hMSCs in EMR-cell compositions over 0 to 10 days. Data points of EMR-cell compositions that were prepared by curing using an Irgacure catalyst are depicted as circles. Data points of EMR-cell compositions that were prepared by curing using a LAP catalyst are depicted as squares.

FIG. 9A: Depicts live/dead stains of hMSCs in LAP EMR scaffolds at Day 3. Green staining indicates live cells; red staining indicates dead cells.

FIG. 9B: Depicts live/dead stains of hMSCs in LAP EMR scaffolds at Day 8. Green staining indicates live cells; red staining indicates dead cells.

FIG. 9C: Depicts a comparison of the viability of hMSCs and HDFs in EMR-cell compositions at day 3. Both EMR-cell compositions were prepared by curing using a LAP catalyst.

FIG. 10A: Scheme depicting how acrylate-PEG-RGD may be incorporated into an EMR of the invention.

FIG. 10B: Scheme depicting how acrylate-PEG-DGR may be incorporated into an EMR of the invention.

FIG. 11A: Histological section after 5 days of implantation showing the EMR-cell composition filling the wound void and cell penetration.

FIG. 11B: Histological section after 21 days of implantation, showing that the EMR-cell composition has degraded, and the skin, with nascent hair follicles, is restored.

FIG. 11C: Image of a wound 5 days after wounding, showing the presence of the EMR-cell composition in the still-open wound.

FIG. 11D: Image of a wound 21 days after wounding, showing that the EMR-cell composition has degraded and the wound has fully closed with minimal scarring.

FIG. 12: Illustration of the encapsulation of cells into an EMR material.

FIG. 13 shows a comparison of the tissue inhibitor of metallopeptidase 1 (“TIMP 1”), tissue inhibitor of metallopeptidase 2 (“TIMP 2”), and vascular endothelial growth factor (“VEGF”) of EMR-cell compositions comprising three different donor cell lines (“SC-EMR Donor 1,” “SC-EMR Donor 2,” and “SC-EMR Donor 3”). FIG. 13 also shows the healing of third degree burns 14 days after implanting a control EMR (“acellular EMR,” an EMR that is unfunctionalized and does not have encapsulated cells) into the murine model. The healing of the acellular EMR was compared to the healing of third degree burns 14 days after implantation into the murine model of EMR-cell compositions comprising the three different donor cell lines at two different concentrations (100 thousand cells and 1 million cells).

FIG. 14 shows hematoxylin and eosin (H&E) histology of after day 5 of implantation into a third degree burn murine model of a control EMR (“acellular EMR,” an EMR that is unfunctionalized and does not have encapsulated cells), implantation of the EMR-cell composition comprising the cells of Donor 1 at a concentration of 100 thousand cells (“SC-EMR Donor 1, 100K cells”), the EMR-cell composition comprising the cells of Donor 1 at a concentration of 1 million cells (“SC-EMR Donor 1, 1M cells”), the EMR-cell composition comprising the cells of Donor 2 at a concentration of 100 thousand cells (“SC-EMR Donor 2, 100K cells”), the EMR-cell composition comprising the cells of Donor 2 at a concentration of 1 million cells (“SC-EMR Donor 2, 1M cells”), the EMR-cell composition comprising the cells of Donor 3 at a concentration of 100 thousand cells (“SC-EMR Donor 3, 100K cells”), and the EMR-cell composition comprising the cells of Donor 3 at a concentration of 1 million cells (“SC-EMR Donor 3, 1M cells”).

FIG. 15 shows H&E histology of after day 14 of implantation into a third degree burn murine model of acellular EMR; SC-EMR Donor 1, 100K cells; SC-EMR Donor 1, 1M cells; SC-EMR Donor 2, 100K cells; SC-EMR Donor 2, 1M cells; SC-EMR Donor 3, 100K cells; and SC-EMR Donor 3, 1M cells.

FIG. 16 shows HLA-A (hu) histology of after day 5 of implantation into a third degree burn murine model of acellular EMR; SC-EMR Donor 1, 100K cells; SC-EMR Donor 1, 1M cells; SC-EMR Donor 2, 100K cells; SC-EMR Donor 2, 1M cells; SC-EMR Donor 3, 100K cells; and SC-EMR Donor 3, 1M cells.

FIG. 17 shows HLA-A (hu) histology of after day 14 of implantation into a third degree burn murine model of acellular EMR; SC-EMR Donor 1, 100K cells; SC-EMR Donor 1, 1M cells; SC-EMR Donor 2, 100K cells; SC-EMR Donor 2, 1M cells; SC-EMR Donor 3, 100K cells; and SC-EMR Donor 3, 1M cells.

FIG. 18 shows MT histology of after day 14 of implantation into a third degree burn murine model of acellular EMR; SC-EMR Donor 1, 100K cells; SC-EMR Donor 1, 1M cells; SC-EMR Donor 2, 100K cells; SC-EMR Donor 2, 1M cells; SC-EMR Donor 3, 1K cells; and SC-EMR Donor 3, 1M cells.

FIG. 19 shows proliferation (WST-1) of hMSCs and HDFns in a two dimensional culture (i.e., standard tissue culture techniques in flasks or wells) and a three dimensional culture (i.e., an EMR scaffold) after exposure to UV light and after exposure to blue light.

FIG. 20 compares the proliferation of fibroblasts and hMSCs after no exposure, after exposure to blue light (415 nm), and after exposure to UV light (365 nm), each for 30 minutes.

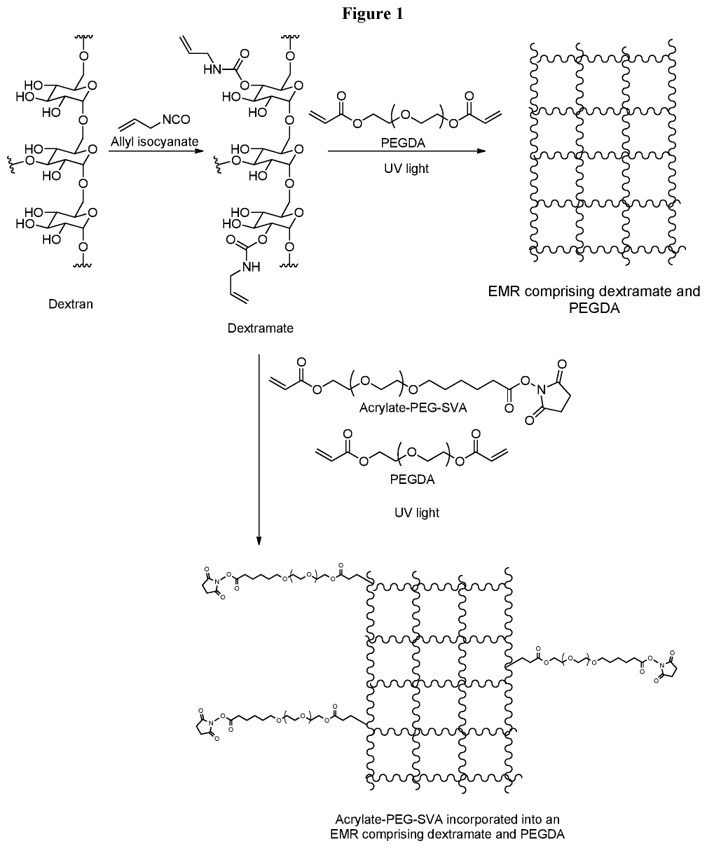

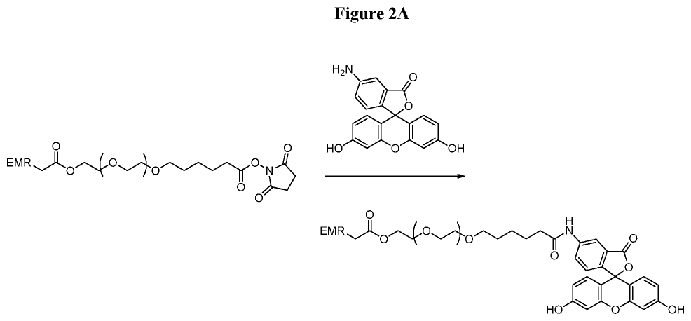

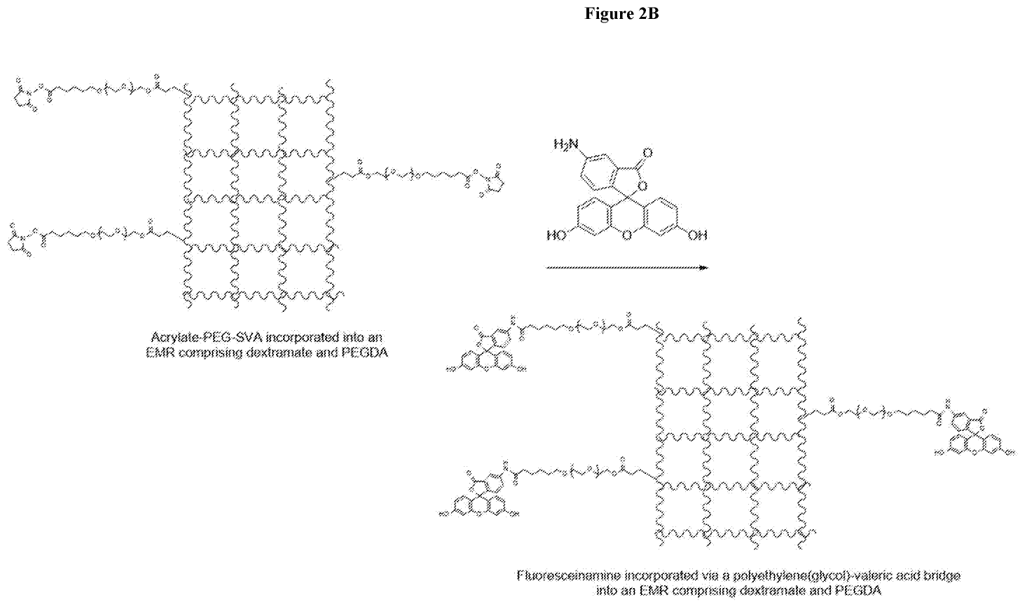

### DETAILED DESCRIPTION OF THE INVENTION

Described herein are EMRs, biocompatible hydrogels, that are useful in wound healing biomaterials because of their unique properties including physiochemical tunability, biocompatibility, degradability, and hydrophilicity. The EMRs described herein may also comprise cells, e.g., stem cells. These properties allow the EMRs to provide a moist environment to the wound bed, promote autolysis, and adsorb wound exudate while functioning as a barrier to further trauma. The EMRs described herein provide a scaffold that stimulates cell infiltration, elicits an early and efficient inflammatory response, and recruits the necessary cell types for rapid revascularization and granulation tissue formation with complete scar-free skin regeneration and hair regrowth. Other applications for the invention include bone, nerve, or adipocyte regeneration, treatment of skin blistering disorders, burn would healing, and cosmetic applications such as skin wrinkles and augmentation surgery.

The EMRs described herein are made from at least one polymer. The EMRs are prepared by mixing one or more polymerizable entities (e.g., unfunctionalized glucans, e.g., unfunctionalized dextrans, and functionalized glucans, e.g. functionalized dextrans, and acrylate-comprising compounds) in aqueous solution. These polymerizable entities are then cured using UV light and/or visible light to generate the EMR. In some embodiments, once the EMR is cured, it is swelled in water, packaged, and sterilized. Clinicians can then apply the EMR as a patch by cutting it to size and placing it directly in the wound bed. In addition the EMRs of this invention comprising cells may be prepared by mixing one or more polymerizable entities (e.g., unfunctionalized glucans, e.g., unfunctionalized dextrans, and functionalized glucans, e.g. functionalized dextrans, and acrylate-comprising compounds) and cells, e.g., stem cells, in an appropriate solution, e.g., a cell culture medium or a saline solution, and then cured. The solution comprising the polymerizable entities and cells may be a high viscosity solution and the high-viscosity solution may be applied to a wound to fill the woundbed and then cured in the wound.

### EMRs Comprising Drugs

The EMRs of this invention enhance wound healing. In some embodiments the EMRs of this invention are combined with a pharmaceutically active compound, which compound enhances wound healing. Pharmaceutically active compounds that are suitable for use in EMRs of the invention described herein include compounds that are suitable for administration to a subject and provides a biological activity or other direct effect that enhances wound healing, e.g., small molecules/drugs, including an angiotensin receptor blocker (ARB), antibiotics, and analgesics.

The EMR and the drugs can be combined in different ways. In some embodiments the drug is trapped within the interstices of the EMR, producing an “interstitial” EMR-drug composition. In other embodiments, the drug is covalently bound to the EMR, producing an EMR-drug conjugate.

In an embodiment of this invention, the drug of the interstitial EMR-drug composition is any pharmaceutically active compound that promotes wound healing, prevents wound infection, and/or provides pain relief. Examples of suitable pharmaceutically active compounds for use in the interstitial EMR-drug compositions are small molecules, e.g., angiotensin receptor blockers (ARBs), antibiotics, and analgesics. Examples of suitable ARBs are valsartan, olmesartan, azilsartan, eprosartan, candesartan, telmisartan, carboxylosartan, losartan, and irbesartan. Suitable examples of antibiotics are penicillins, cephalosporins, sulfonamides, tetracyclines, aminoglycosides, glycopeptides, and macrolides. Examples of suitable analgesics are non-steroidal inflammatory drugs (e.g., aspirin, salicylic acid, ketorolac, diclofenac, indomethacin, ibuprofen, ketoprofen, and naproxen), opioids, opiates, gabapentin, and pre-gabalin.

In an embodiment of this invention, the drug of the EMR-drug conjugate is any pharmaceutically active compound that comprises a carboxylic acid group. Examples of such pharmaceutically active compounds include small molecules, e.g. angiotensin receptor blockers (ARBs), antibiotics, and analgesics that comprise a carboxylic acid group. Examples of ARBs comprising a carboxylic acid group are valsartan, olmesartan, azilsartan, eprosartan, candesartan, telmisartan, carboxylosartan, and irbesartan. Examples of suitable antibiotics comprising a carboxylic acid group are penicillins and cephalosporins. Examples of analgesics comprising a carboxylic acid group are gabapentin, pre-gabalin, aspirin, salicylic acid, ketorolac, diclofenac, indomethacin, ibuprofen, ketoprofen, and naproxen.

In an embodiment of this invention, the drug of the EMR-drug conjugate is any pharmaceutically active compound that comprises a functional group (e.g., an alcohol group, an ester group) that can be transformed into a carboxylic acid group. Examples of pharmaceutically active compounds that comprise a functional group that can be transformed into a carboxylic acid group are ARBs (e.g., losartan).

In an embodiment of this invention, the drug of the interstitial EMR-drug composition or the EMR-drug conjugate is an ARB, e.g., valsartan, olmesartan, azilsartan, eprosartan, candesartan, telmisartan, carboxylosartan, irbesartan, losartan, or mixtures thereof. In an embodiment of this invention, the interstitial EMR-drug composition or the EMR-drug conjugate comprising an ARB accelerates wound closure.

In an embodiment, the interstitial EMR-drug composition or EMR-drug conjugate of the invention closely match the physical properties of unfunctionalized EMRs, including the swelling properties, the stiffness, the porosity, and the oxygen permeability. Suitable examples of unfunctionalized EMRs, as well as methods for preparing and characterizing unfunctionalized EMRs may be found in, e.g., U.S. Pat. Nos. 8,900,868 and 9,655,844, U.S. Pre-Grant Publication Nos. 2013/0102531 and 2015/0174154, and Sun et al., PNAS 2011, 108, 20976-20981 and Shen et al., Acellular Hydrogels for Regenerative Burn Wound Healing: Translation from a Porcine Model, Journal of Investigative Dermatology 2015. The properties of the EMR-drug conjugates, like the properties of unfunctionalized EMRs, are determined through conventional methods. See, e.g., Sun et al., PNAS 2011, 108, 20976-20981, which is incorporated in its entirety by reference.

For example, the swelling ratios of the interstitial EMR-drug compositions or EMR-drug conjugates are determined via gravimetric analysis to evaluate the capacity of the interstitial EMR-drug compositions or EMR-drug conjugates to absorb water (as a surrogate for wound exudate) and desorb water (as a surrogate for wound hydration). A moist environment promotes autolytic debridement.

As another example, critical stiffness of the EMR is required to maintain the integrity of the wound bed for the healing of self-contracting wounds without compromising the properties of the EMR. Standard mechanical measurements of the elastic modulus are collected for the interstitial EMR-drug compositions or EMR-drug conjugates using a rheometer. A suitable shear storage modulus for the interstitial EMR-drug compositions or EMR-drug conjugates of the invention is between about 10 Pa and about 2000 Pa, between about 10 Pa and about 1500 Pa, between about 10 Pa and about 1000 Pa, between about 10 Pa and about 500 Pa, and between about 10 Pa and about 250 Pa.

As another example, the porosity and morphology of the EMRs, which are directly related to the crosslinking density, affects cell infiltration, tissue ingrowth and degradation rate of the polymers of the EMR. The morphology and porosity of all interstitial EMR-drug compositions or EMR-drug conjugates can be determined using scanning electron microscopy to provide a representative view of the microstructure of the interstitial EMR-drug compositions or EMR-drug conjugates. A suitable oxygen permeability for the interstitial EMR-drug compositions or EMR-drug conjugates is full oxygen permeability under normoxic conditions.

As another example, oxygen transfer facilitates wound healing because it increases granulation tissue formation, epithelialization, and fibroblast recruitment and promotes wound contraction. The interstitial EMR-drug compositions or EMR-drug conjugates must be oxygen permeable to enhance wound healing and prevent anaerobic bacterial infections, which is thought to occur at the interface of the wound bed and oxygen impermeable wound dressings. The rate of oxygen transfer through hydrated interstitial EMR-drug compositions or EMR-drug conjugates of various thicknesses is measured continuously using oxygen sensors under both physiological and hypoxic conditions. Monitoring oxygen permeability over time provides a temporal profile of oxygen transfer through the EMR.

In all of these characterization steps, the interstitial EMR-drug compositions or EMR-drug conjugates of the invention are compared to unfunctionalized EMR as a control. Unfunctionalized EMRs are well-characterized and produced by known methods. See, e.g., U.S. Pat. Nos. 8,900,868 and 9,655,844, incorporated herein in their entirety. See also U.S. Pre-Grant Publication Nos. 2013/0102531 and 2015/0174154, incorporated herein in their entirety. See also Sun et al., PNAS 2011, 108, 20976-20981 and Shen et al., Acellular Hydrogels for Regenerative Burn Wound Healing: Translation from a Porcine Model, Journal of Investigative Dermatology 2015, incorporated herein in their entirety. The properties of the unfunctionalized EMRs include an elastic modulus of approximately 1.5 kPa, pore sizes of ˜10 μM, and full oxygen permeability under normoxic conditions.

The interstitial EMR-drug composition or the EMR-drug conjugate is applied to a chronic wound (e.g., a diabetic ulcer), which: (1) provides mechanical support by mimicking the mechanical properties of tissue; (2) increases the rate of wound healing and closure by increasing cell migration and revascularization of the wound site; and (3) enhances the rate of wound healing and closure, as compared to the EMR without the drug, by releasing the drug into the wound for a prolonged period.

Wound healing involves the mediation of many initiators that are critical for tissue repair. For example, fibroblasts are recruited to the wound area, where they proliferate and aid in remodeling the extracellular environment and wound contraction.

A scratch wound assay may be used to evaluate basic fibroblast cell recruitment induced by interstitial EMR-drug compositions or EMR-drug conjugates. Briefly, fibroblast cells are grown to confluence and a thin wound is created by scratching the monolayer with a pipette tip. The interstitial EMR-drug composition or EMR-drug conjugate is then applied to the thin wound at various time points to assess its effect on fibroblast cell recruitment. For example, the EMR-drug conjugate or interstitial EMR-drug composition may be applied immediately after scratching the fibroblasts, or application of the EMR-drug conjugate or interstitial EMR-drug composition may be delayed for minutes or hours thereafter. The migratory speed and number of fibroblasts infiltrating the wound area in the presence of varying amounts of the EMR-drug conjugate or interstitial EMR-drug compositions are counted using time lapse light microscopy and compared to controls containing no EMR or an unfunctionalized EMR. Together, these data provide insight into the ability of the EMR-drug conjugate or interstitial EMR-drug compositions to promote fibroblast recruitment. Furthermore, any early indicators of potential cytotoxicity of the EMR-drug conjugate or interstitial EMR-drug compositions are discovered in these assays. This assay can also be performed with mammalian endothelial cells (e.g., human endothelial cells), which are essential for the neovascularization of the wound bed to provide critical nutrients and oxygen to the damaged tissue.

Cytotoxicity of the EMR-drug conjugates or interstitial EMR-drug compositions may be evaluated by culturing human fibroblasts with the EMR-drug conjugates or interstitial EMR-drug compositions of the invention in wells and then quantifying fibroblast viability, morphology, and proliferation in the presence of the EMR-drug conjugates or interstitial EMR-drug compositions of the invention. Cytotoxicity of the EMR-drug conjugates or interstitial EMR-drug compositions may also be evaluated by culturing other cell types (e.g., keratinocytes) with the EMR-drug conjugates and interstitial EMR-drug compositions of this invention in wells and then quantifying the viability, morphology, and proliferation of these other cell types in the presence of EMR-drug conjugates and interstitial EMR-drug compositions of this invention.

Cell morphology, fibroblast viability, and proliferation may be analyzed by any well-known method in the art, e.g., cell morphology may be analyzed via light microscopy, to identify any structural changes to the cell, and the fibroblast viability may be confirmed via a two-color fluorescence cell viability assay that visualizes both viable and non-viable cells using fluorescence microscopy or flow cytometry.

Fibroblast proliferation may be measured using a WST assay (commercially available WST-1 Cell Proliferation Assay kits are available from Cayman Chemical).

The EMR-drug conjugates and interstitial EMR-drug compositions of the invention stay intact for long enough to provide sufficient mechanical support for cells to migrate into the wound bed but degrade sufficiently to allow sustained drug release, further cell invasion, and tissue regeneration. In vitro degradation of the EMR in the EMR-drug conjugates and interstitial EMR-drug compositions is quantified using a neutrophil-like cell suspension using HL-60s differentiated in DMSO for five days is used. Samples of the inventive interstitial EMR-drug compositions or EMR-drug conjugates are incubated in these neutrophil-like cultures for about 12, 24, 36, 48, 60, and 72 hours, or anytime there between, after which the washed EMR-drug conjugates or interstitial EMR-drug compositions are lyophilized, and the initial dry weight of the EMR-drug conjugates or interstitial EMR-drug compositions are compared to the post-degraded dry weight of the EMR-drug conjugates or interstitial EMR-drug compositions. This provides the culture-based degradation kinetics for each formulation of interstitial EMR-drug composition or EMR-drug conjugates. Drug release is also evaluated in this experiment, and the amount of drug released into the culture media is quantified by HPLC/MS (high performance liquid chromatography/mass spectrometry) or ELISA. Methods for quantifying drugs via HPLC/MS are well known in the art.

For assaying and comparing the release kinetics of the drug from EMR-drug conjugates or interstitial EMR-drug compositions in cell culture, a panel of endogenous enzymes that are expected to release the drug from the EMR scaffold is selected. This panel of enzymes includes, e.g., proteinases, proteases, esterases, and glycosidases such as those that have known roles in extracellular matrix remodeling during wound healing (e.g., matrix metalloproteinase, gelatinases and collagenases). The EMR-drug conjugates or interstitial EMR-drug compositions are incubated with a single enzyme or with a mixture of enzymes selected from the panel of enzymes. Drug release is measured over time on incubation of the EMR-drug conjugates or interstitial EMR-drug compositions with the single enzymes or mixture of enzymes.

Without wishing to be bound by theory, it is contemplated that the drug component of the EMR-drug conjugates or interstitial EMR-drug compositions is released as the polymers of the EMR component of the EMR-drug conjugates or interstitial EMR-drug compositions are digested by endogenous enzymes at the wound site. The effectiveness of treating a wound with the EMR-drug conjugates or interstitial EMR-drug compositions may be measured using methods known in the art and may be compared to a control, e.g., treating the wound with the drug alone (e.g., an ARB not combined with an EMR) and an EMR without the drug.

The EMR-drug conjugates and interstitial EMR-drug compositions of the invention ensure patient compliance. Adherence to a regimen of cleaning and applying a dressing or drug to a wound, as is required in conventional would treatment, is difficult for diabetic patients in poor health. The EMR-drug conjugates and inventive interstitial EMR-drug compositions of the invention provide mechanical support to the wound, increase cell migration and revascularization of the wound site, and provide drug release into the wound for a prolonged time period. The prolonged time period is at least 5 days, preferably at least 7 days, preferably at least 8 days, preferably at least 10 days, preferably at least 12 days, preferably at least 14 days, preferably at least 20 days. Thus, with the EMR-drug conjugates and inventive interstitial EMR-drug compositions of the invention, patients do not have to clean and apply a dressing or drug to a wound daily, which leads to increased patient compliance and faster healing.

This invention provides a method for preparing the interstitial EMR-drug composition of the invention, comprising: (a) mixing a pharmaceutically active compound and a polymerizable entity; and (b) curing the mixture of step (a) with UV light and/or visible light. Examples of suitable polymerizable entities include unfunctionalized glucans (e.g., unfunctionalized dextrans) and functionalized glucans (e.g., unfunctionalized dextrans) and functionalized dextrans as described herein.

In one embodiment of the method for preparing the interstitial EMR-drug composition of the invention, the pharmaceutically active compound and the polymerizable entity are mixed together with a cross-linking catalyst selected from a UV-crosslinking catalyst or a visible light-cross-linking catalyst. Examples of suitable UV-crosslinking catalysts are Irgacure catalysts (e.g., Irgacure 2959) and lithium phenyl-2,4,6-trimethylbenzoylphosphinate (LAP). An example of a suitable visible-light cross-linking catalysts is eosin-Y.

In one embodiment of the method for preparing the interstitial EMR-drug composition of the invention, the pharmaceutically active compound, the polymerizable entity, and the cross-linking catalyst are further mixed together with an acrylate-comprising compound (e.g., polyethylene(glycol)diacrylate, polyethylene(glycol)acrylate, and acrylate-polyethylene(glycol)-succinimidyl valeric acid).

This invention provides a method for preparing the EMR-drug conjugate of the invention, comprising: (a) conjugating a pharmaceutically active compound with a polymerizable entity; and (b) curing the product of step (a) with UV light and/or visible light. Examples of suitable polymerizable entities include unfunctionalized glucans (e.g., unfunctionalized dextrans) and functionalized glucans (e.g., functionalized dextrans) as described herein.

In one embodiment of the method for preparing the EMR-drug conjugate of the invention, the pharmaceutically active compound and the polymerizable entity are mixed together with a cross-linking catalyst selected from a UV-crosslinking catalyst or a visible light-crosslinking catalyst. Examples of suitable UV-crosslinking catalysts are Irgacure catalysts (e.g., Irgacure 2959) and lithium phenyl-2,4,6-trimethylbenzoylphosphinate (LAP). An example of a suitable visible-light cross-linking catalysts is eosin-Y.

In one embodiment of the method for preparing the EMR-drug conjugate of the invention, the pharmaceutically active compound, the polymerizable entity, and the cross-linking catalyst are further mixed together with an acrylate-comprising compound (e.g., polyethylene(glycol)diacrylate, polyethylene(glycol)acrylate, and acrylate-polyethylene(glycol)-succinimidyl valeric acid).

In one embodiment of the method for preparing the interstitial EMR-drug composition or the EMR-drug conjugate of the invention, the polymerizable entity is unfunctionalized glucan. Suitable unfunctionalized glucans that may be used in the interstitial EMR-drug compositions or the EMR-drug conjugates of the invention have a molecular weight range between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The molecular weight may be number average or weight average.

In one embodiment of the method for preparing the interstitial EMR-drug composition or the EMR-drug conjugate of the invention, the polymerizable entity is unfunctionalized dextran, which has the following structure:

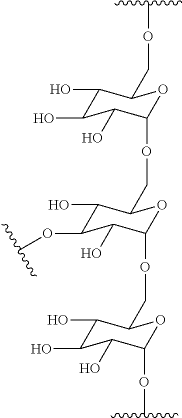 [SMILES: *OCC1OC(OCC2OC(OCC3OC(O*)C(O)C(O)C3O)C(O)C(O*)C2O)C(O)C(O)C1O]

Suitable unfunctionalized dextrans that may be used in the interstitial EMR-drug compositions or the EMR-drug conjugates of the invention have a molecular weight range between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The molecular weight may be number average or weight average.

In one embodiment of the method for preparing the interstitial EMR-drug composition or the EMR-drug conjugates of the invention, the polymerizable entity is a functionalized glucan. Suitable functionalized glucans are glucans that are functionalized with polymerizable compounds, such as ethylamine, allyl carbamate, and mixtures thereof. Suitable molecular weight ranges for functionalized glucans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. In a preferred embodiment of the invention the polymerizable compound is allyl carbamate.

For use in the interstitial EMR-drug compositions and EMR-drug conjugates of the invention, suitable functionalized glucans have a degree of substitution between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the functionalized glucans have a degree of substitution between about 0.05 and about 0.5. In another preferred embodiment, the functionalized glucans have a degree of substitution between about 0.05 and about 0.25.

In one embodiment of the method for preparing the interstitial EMR-drug composition or the EMR-drug conjugates of the invention, the polymerizable entity is a functionalized dextran. Suitable functionalized dextrans are dextrans that are functionalized with polymerizable compounds, such as ethylamine, allyl carbamate, and mixtures thereof. Suitable molecular weight ranges for functionalized dextrans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. In a preferred embodiment of the invention the polymerizable compound is allyl carbamate.

For use in the interstitial EMR-drug compositions and EMR-drug conjugates of the invention, suitable functionalized dextrans have a degree of substitution between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the functionalized dextrans have a degree of substitution between about 0.05 and about 0.5. In another preferred embodiment, the functionalized dextrans have a degree of substitution between about 0.05 and about 0.25.

In a preferred embodiment of the interstitial EMR-drug compositions and EMR-drug conjugates of the invention, the polymerizable entity is dextramate. Dextramate is a dextran that has been reacted with allyl isocyanate molecules to produce a dextran that is functionalized with allyl carbamate groups. In some embodiments of the invention, the degree of substitution in the dextramate is about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. The degree of substitution refers to the degree of hydroxyl groups on dextran that have reacted with allyl isocyanate molecules (i.e., the degree of hydroxyl groups on dextran that are substituted with allyl carbamate groups). In a preferred embodiment, the degree of substitution in dextramate is between about 0.05 and about 0.5. In another preferred embodiment, the degree of substitution in dextramate is between about 0.05 and about 0.25.

For use in the interstitial EMR-drug compositions and EMR-drug conjugates of the invention, suitable acrylate-comprising compounds have a molecular weight range between about 100 Da and about 250,000 Da, between about 150 Da and about 100,000 Da, between about 200 Da and about 50,000 Da, between about 250 Da and about 25,000 Da, between about 300 Da and about 10,000 Da, and between about 350 Da and about 5,000 Da. In one preferred embodiment, the acrylate-comprising compounds have a molecular weight range between about 1,000 Da and about 5,000 Da. In another preferred embodiment, the acrylate-comprising compounds have a molecular weight range between about 2,000 Da and about 4,000 Da.

One embodiment of the invention is an interstitial EMR-drug composition or an EMR-drug conjugate wherein the EMR component comprises a mixture of a functionalized dextran and an acrylate-comprising compound, in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

One preferred embodiment of the invention is an interstitial EMR-drug composition or an EMR-drug conjugate wherein the EMR component comprises a mixture of dextramate and PEGDA in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

One embodiment of the invention is an interstitial EMR-drug composition or an EMR-drug conjugate wherein the drug component makes up about 0.001% to about 25% of the total weight of the interstitial EMR-drug composition or an EMR-drug conjugate, about 0.01% to about 10% of the total weight of the interstitial EMR-drug composition or an EMR-drug conjugate, or about 0.1% to about 5% of the total weight of the interstitial EMR-drug composition or an EMR-drug conjugate.

In one embodiment, the EMR-drug conjugate has the following formula (I), wherein a pharmaceutically active compound is covalently linked to the EMR component:

Q-X—Y (I),

Q is the pharmaceutically active compound, X is a linker group, and Y is an EMR.

One embodiment of the invention is an EMR-drug conjugate of formula (I) wherein the pharmaceutically active compound is a small molecule comprising a carboxylic acid group.

This invention provides a method for preparing the EMR-drug conjugate of the invention, comprising:

(a) reacting a small molecule of formula (XVIII) with a compound of formula (XIXa) or formula (XIXb) to yield a compound of formula (XXa) or (XXb):

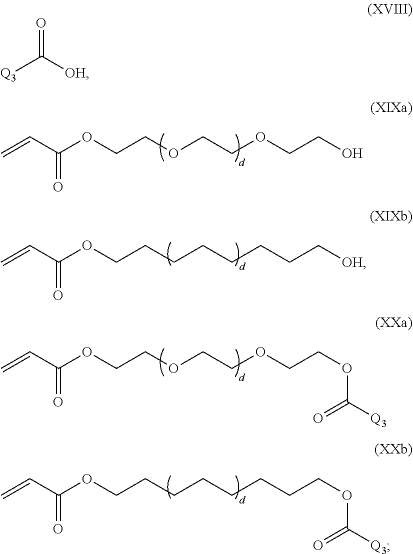

wherein Q₃ is a residue of any small molecule (e.g., a small molecule selected from the group consisting of ARBs, antibiotics, and analgesics) and wherein d is 0 or an integer between 1 and 500, between 1 and 250, between 1 and 200, between 1 and 100, between 1 and 50, or between 1 and 10;

(b) reacting the compound of formula XXa or formula XXb with acryl carbamate groups on a dextran of formula X:

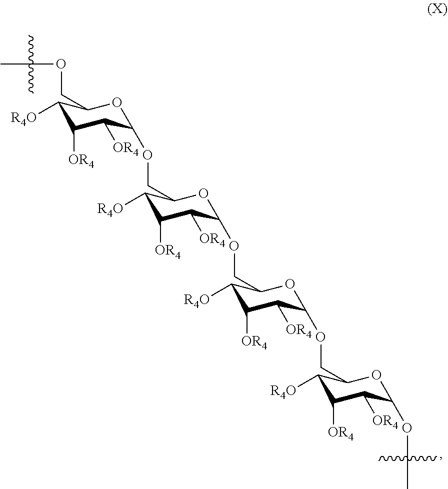

wherein R₄ is H, allyl carbamate, or mixtures thereof;

(c) optionally adding to the product of step (b) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and

(d) curing the product of step (b) or step (c) with UV light and/or visible light.

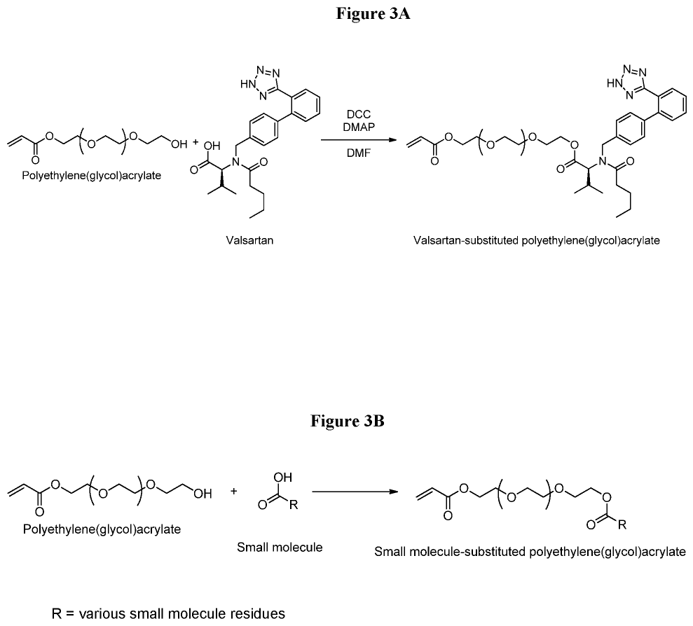

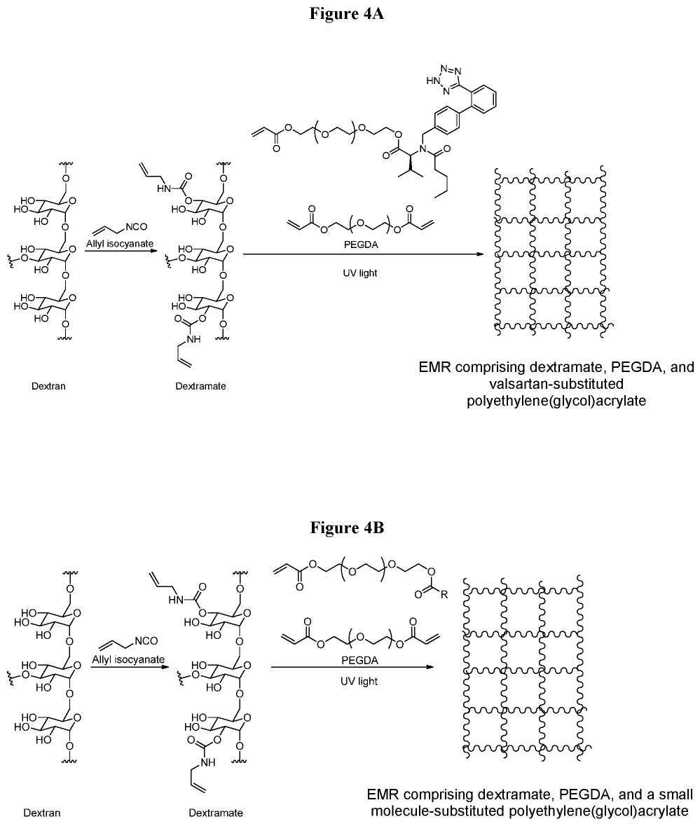

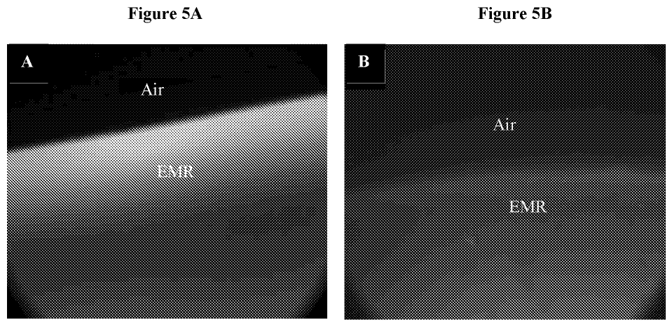

Step (b) of this method is illustrated in FIG. 3B. Steps (c) and (d) of this method are illustrated in FIG. 4B.

In one preferred embodiment of the method, Q₃ in formula (XVIII) is selected from the group consisting of

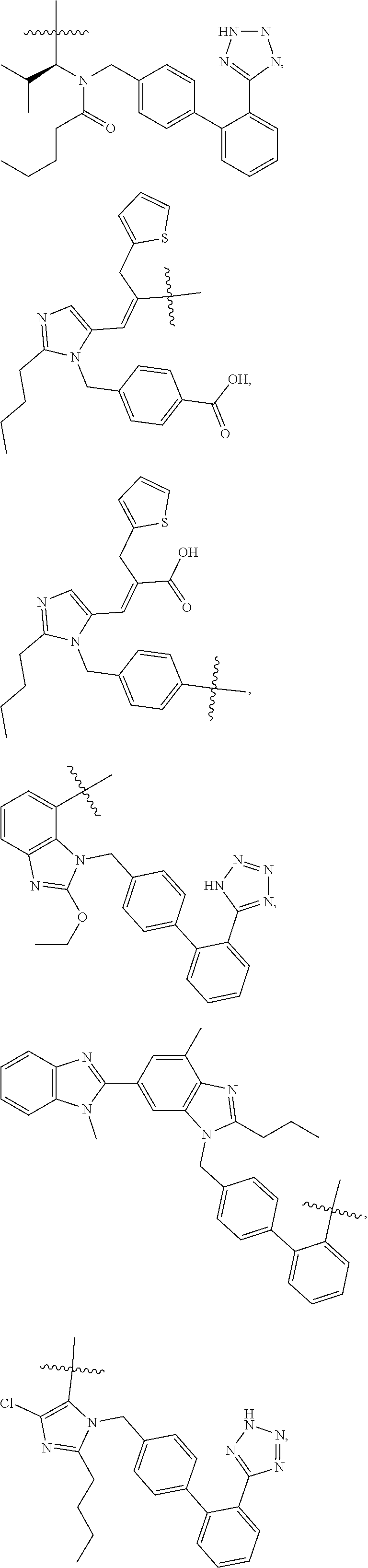

and mixtures thereof.

One embodiment of the invention is an EMR-drug conjugate of formula (I) wherein the pharmaceutically active compound is an ARB is selected from the group consisting of valsartan, olmesartan, azilsartan, eprosartan, candesartan, telmisartan, carboxylosartan, irbesartan, losartan, and mixtures thereof.

Another embodiment of the invention is an EMR-drug conjugate of formula (I) wherein the linker group is selected from the group consisting of: a bond,

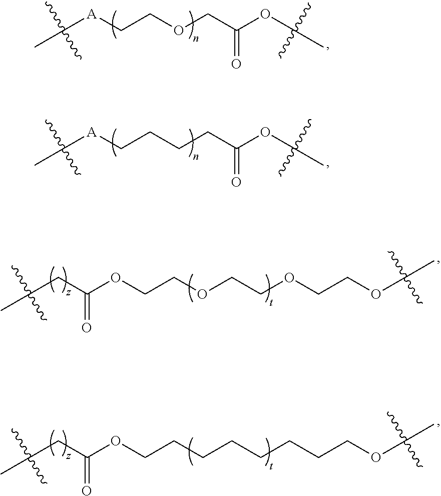

and mixtures thereof, wherein A is O, S, or NH, n is 0 or an integer between 1 and 500, between 1 and 250, between 1 and 200, between 1 and 100, between 1 and 50, or between 1 and 10; t is 0 or an integer between 1 and 500, between 1 and 250, between 1 and 200, between 1 and 100, between 1 and 50, or between 1 and 10; and z is 0 or an integer between 1 and 10.

In one embodiment of the EMR-drug conjugate of formula (I), the pharmaceutically active compound is valsartan.

In some embodiments of the invention, the EMR-drug conjugate further comprises a pharmaceutically active compound that is trapped within the interstices of the EMR-drug conjugate. Examples of suitable pharmaceutically active compounds that may be trapped within the interstices of the EMR-drug conjugate ARBs, antibiotics, and analgesics.

This invention provides a method for preparing the EMR-drug conjugate of the invention, comprising:

(a) transforming an ARB with a structure of formula (IIa) into a compound of formula (IIb):

Q₁-OH (IIa)

Q₁-L (IIb),

wherein Q₁ is selected from the group consisting of:

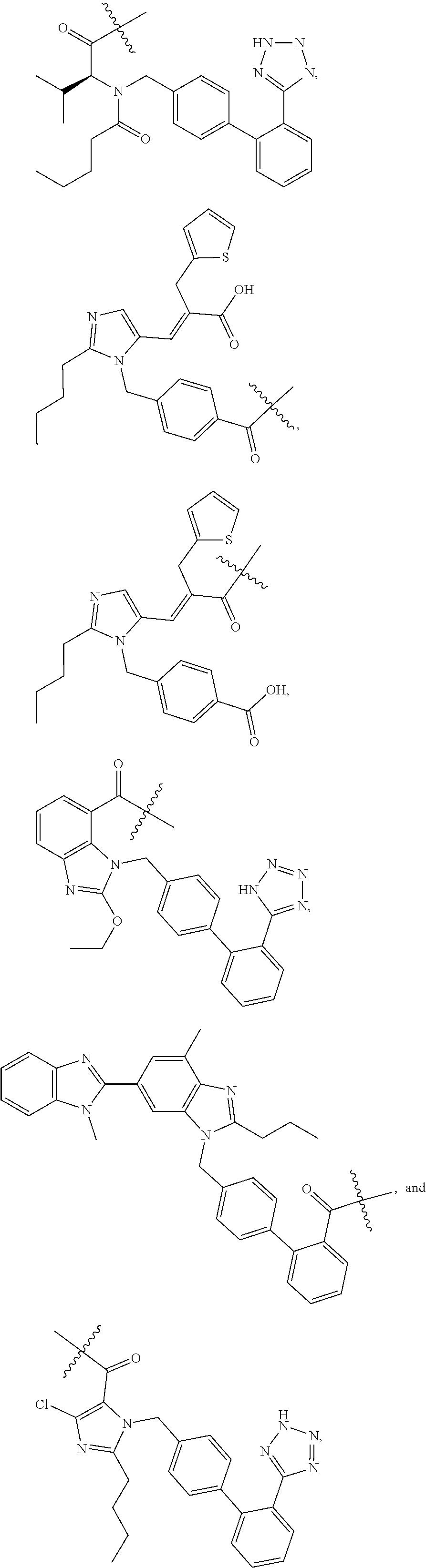

wherein L is selected from the group consisting of: —Cl, —Br, —I, and —OR₁, wherein R₁ is C₁-C₁₀ alkyl or

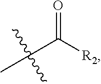

and wherein R₂ is C₁-C₁₀ alkyl;

(b) reacting the compound of formula (IIb) with a compound of formula (Ma) or formula (IIIb) to yield a compound of formula (IVa) or (IVb):

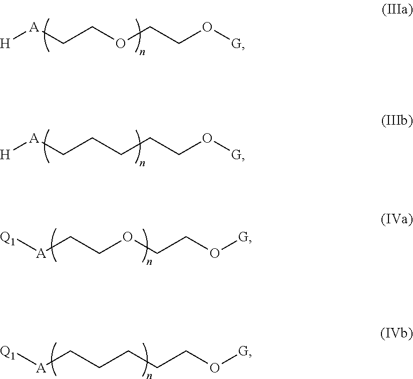

wherein G is a protecting group, A is O, S, or NH, n is 0 or an integer between 1 and 500, between 1 and 250, between 1 and 200, between 1 and 100, between 1 and 50, or between 1 and 10; and Q₁ is the same as defined in step (a);

(c) deprotecting the compound of formula (IVa) or formula (IVb) to yield a compound of formula (Va) or formula (Vb):

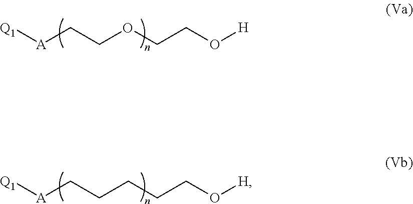

wherein Q₁ is the same as defined in step (a) and A and n are the same as defined in step (b);

(d) oxidizing the compound of formula (IVa) or formula (IVb) to yield a product of formula (VIa) or formula (VIb):

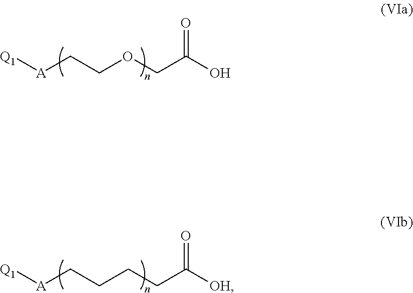

wherein Q₁ is the same as defined in step (a) and A and n are the same as defined in step (b);

(e) transforming the compound of formula (VIa) or formula (VIb) to yield a compound of formula (VIIa) or formula (VIIb):

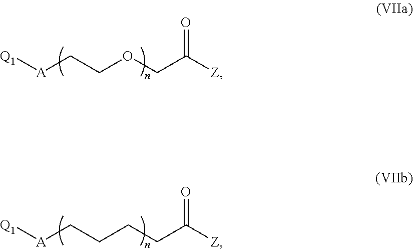

wherein Z is —Cl, —Br, —I, and —OR₃, wherein R₃ is C₁-C₁₀ alkyl, and wherein Q₁ is the same as defined in step (a) and A and n are the same as defined in step (b);

(f) reacting the compound of formula (VIIa) or formula (VIIb) with free hydroxyl groups on a polymerizable entity selected from the group consisting of unfunctionalized dextran, functionalized dextran, and mixtures thereof;

(g) optionally adding to the product of step (f) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and

(h) curing the product of step (f) or step (g) with UV light and/or visible light.

Another embodiment of the invention is a method for preparing the EMR-drug conjugate of the invention, comprising:
(a) oxidizing an ARB with a structure of formula (IIc)

Q₂-CH₂OH (IIc),

wherein Q₂ is

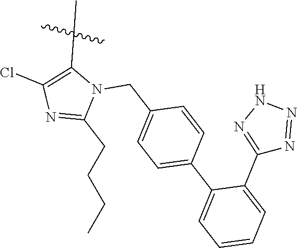 [SMILES: *c1c(Cl)nc(CCCC)n1Cc1ccc(-c2ccccc2-c2nn[nH]n2)cc1]

into a compound of formula (IIa-1):

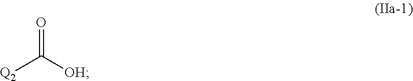

(b) transforming the compound formula (IIa-1) into a compound of formula (IIb-1):

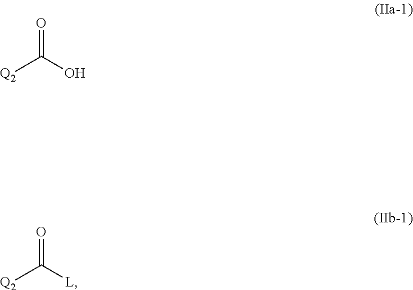

wherein Q₂ is the same as defined in step (a), wherein L is selected from the group consisting of —Cl, —Br, —I, and —OR₁, wherein R₁ is C₁-C₁₀ alkyl or

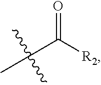

and wherein R₂ is C₁-C₁₀ alkyl;

(c) reacting the compound of formula (IIb-1) with a compound of formula (Ma) or formula (IIIb) to yield a compound of formula (IVa-1) or (IVb-1):

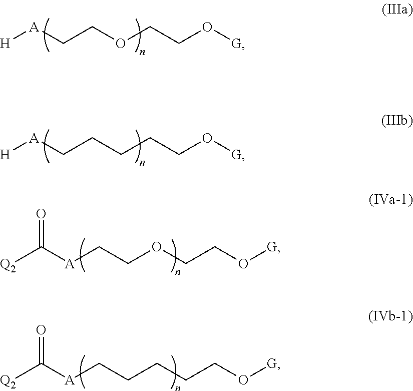

wherein G is a protecting group, A is O, S, or NH, n is 0 or an integer between 1 and 500, between 1 and 250, between 1 and 200, between 1 and 100, between 1 and 50, or between 1 and 10; and Q₂ is the same as defined in step (a)

(d) deprotecting the compound of formula (IVa-1) or formula (IVb-1) to yield a compound of formula (Va-1) or formula (Vb-1):

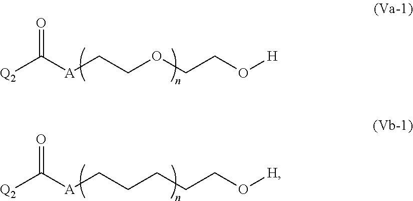

wherein Q₂ is the same as defined in step (a) and A and n are the same as defined in step (c);

(e) oxidizing the compound of formula (IVa-1) or formula (IVb-1) to yield a product of formula (VIa-1) or formula (VIb-1):

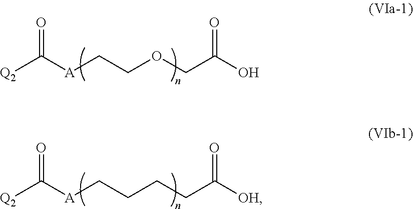

wherein Q₂ is the same as defined in step (a) and A and n are the same as defined in step (c);

(f) transforming the compound of formula (VIa-1) or formula (VIb-1) to yield a compound of formula (VIIa-1) or formula (VIIb-1):

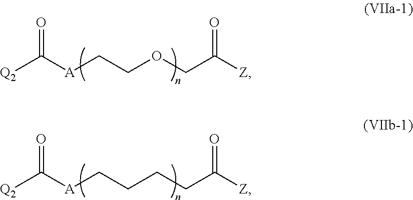

wherein Z is —Cl, —Br, —I, and —OR₃, wherein R₃ is C₁-C₁₀ alkyl, and wherein Q₂ is the same as defined in step (a) and A and n are the same as defined in step (c);

(g) reacting the compound of formula (VIIa-1) or formula (VIIb-1) with free hydroxyl groups on a polymerizable entity selected from the group consisting of unfunctionalized dextran, functionalized dextran, and mixtures thereof;

(h) optionally adding to the product of step (g) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and

(i) curing the product of step (g) or step (h) with UV light and/or visible light.

Another embodiment of the invention is a method for preparing the EMR-drug conjugate of the invention, wherein the protecting group G is selected from the group consisting of C₁-C₁₀ unbranched or branched alkyl; —SiMe₃; —SiEt₃; —Si(iPr)₃; —SiPh₃; —SiMe₂iPr; —SiMe₂Et; —SiEt₂iPr; and —CH₂-Ph, wherein the Ph is unsubstituted or substituted with at least one substituent selected from the group consisting of —OMe, —NO₂, —F, —Cl, —Br, —I, —CF₃, —SiMe₃, and —CN.

In an embodiment of the invention the EMR-drug conjugate comprises a functionalized dextran of formula (X):

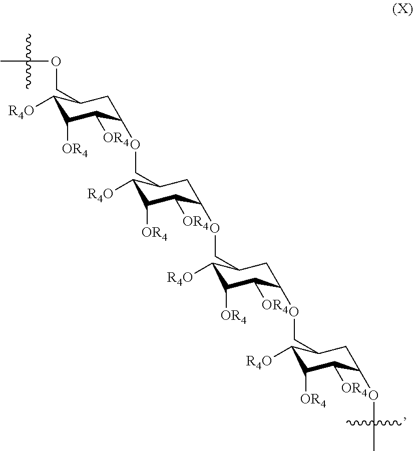

wherein R₄ is H, allyl carbamate, or mixtures thereof.

Another embodiment of the invention is a method for preparing the EMR-drug conjugate of the invention, comprising:

(a) reacting a compound of formula (IIa):

Q₁-OH (IIa),

wherein Q₁ is selected from the group consisting of:

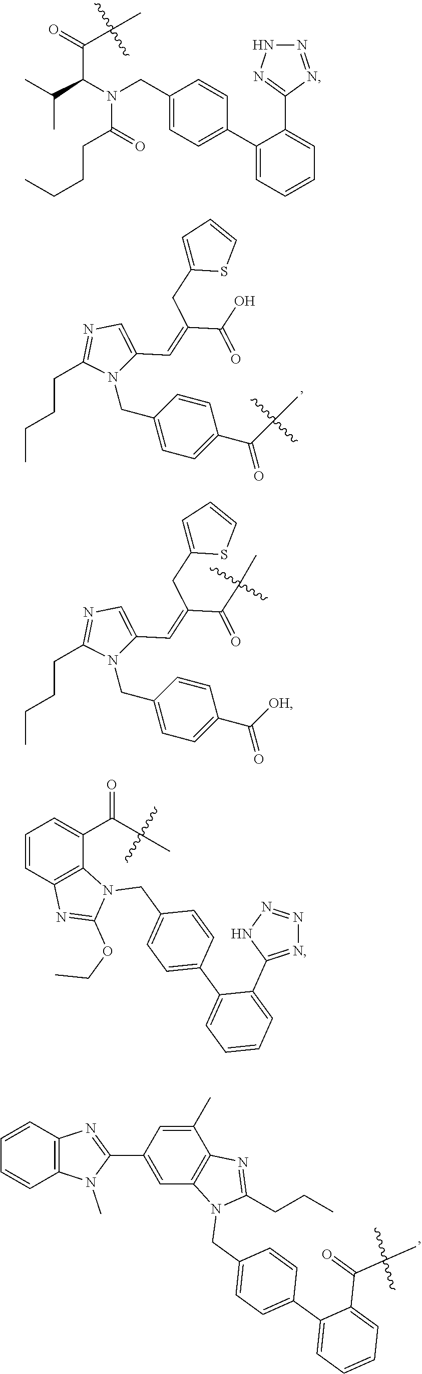

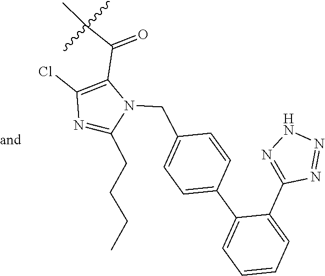 [SMILES: *C(=O)c1c(Cl)nc(CCCC)n1Cc1ccc(-c2ccccc2-c2nn[nH]n2)cc1]

with a structure of formula (XIVa) or formula (XIVb):

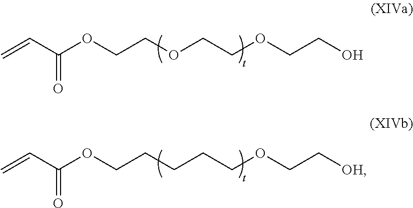

wherein t is 0 or an integer between 1 and 500, between 1 and 250, between 1 and 200, between 1 and 100, between 1 and 50, or between 1 and 10,

to yield a compound of formula (XVa) or (XVb):

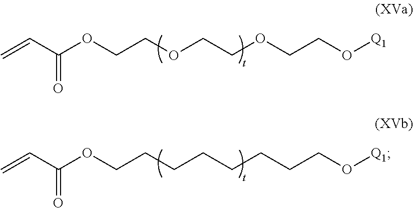

(b) mixing the compound of formula (XVa) or formula (XVb) with a compound of formula (X):

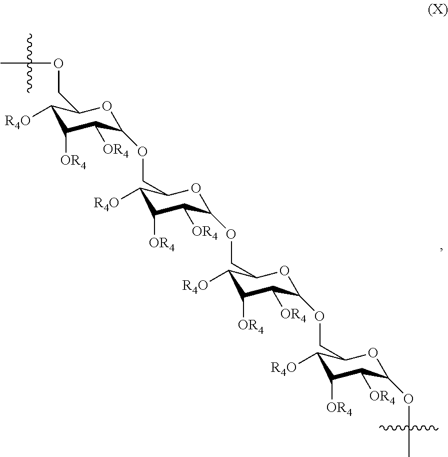

wherein R₄ is H, allyl carbamate, or mixtures thereof;

(c) optionally adding to the product of step (b) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and

(d) curing the product of step (b) or step (c) with UV light and/or visible light.

Another embodiment of the invention is a method for preparing the EMR-drug conjugate of the invention, comprising:

(a) transforming a compound of formula (XIVa) or formula (XIVb):

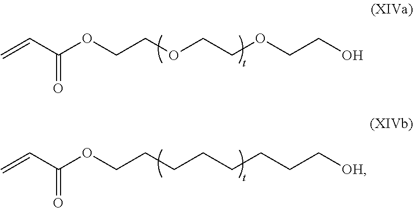

into a compound of formula (XIVa-1) or formula (XIVb-1):

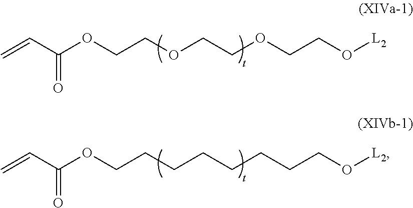

wherein t is 0 or an integer between 1 and 500, between 1 and 250, between 1 and 200, between 1 and 100, between 1 and 50, or between 1 and 10; and L₂ is a group selected from the group consisting of C₁-C₁₀ alkyl or

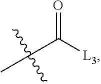

and wherein L₃ is C₁-C₁₀ alkyl;

(b) reacting the compound of formula (XIVa-1) or formula (XIVb-1) with a compound of formula (IIc):

Q₂-CH₂OH (IIc)

to yield a compound of formula (XIVa-2) or formula (XIVb-2):

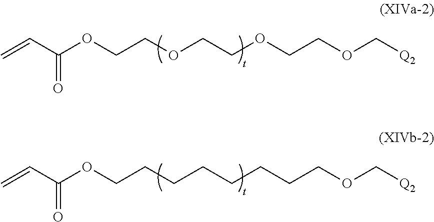

wherein Q₂ is

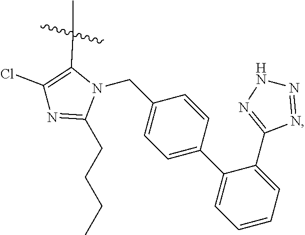 [SMILES: *c1c(Cl)nc(CCCC)n1Cc1ccc(-c2ccccc2-c2nn[nH]n2)cc1]

and wherein t and L₂ are as defined in step (a);

(c) mixing the compound of formula (XIVa-2) or formula (XIVb-2) with a compound of formula (X):

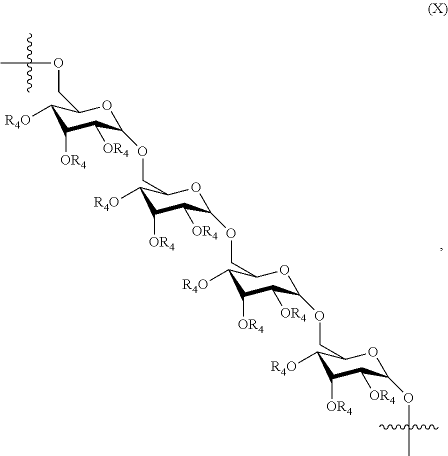

wherein R₄ is H, allyl carbamate, or mixtures thereof; and

(d) optionally adding to the product of step (c) an acrylate-comprising compound selected from the group consisting of polyethylene(glycol)diacrylate, polyethylene(glycol)acrylate, acrylate-polyethylene(glycol)-succinimidyl valeric acid, and mixtures thereof; and

(e) curing the product of step (c) or step (d) with UV light and/or visible light.

Suitable pore sizes for the cured interstitial EMR-drug compositions or EMR-drug conjugates of the invention are about 0.001 microns to about 100 microns, about 5 microns to about 90 microns, about 10 microns to about 80 microns, about 15 microns to about 70 microns, about 20 microns to about 60 microns, or about 25 microns to about 50 microns.

This invention provides a pharmaceutical composition comprising the interstitial EMR-drug composition or the EMR-drug conjugate of the invention and at least one pharmaceutically acceptable excipient.

This invention provides a method of treating wounds, comprising applying to a wound in a patient in need thereof an effective amount of the interstitial EMR-drug composition or the EMR-drug conjugate of the invention. An effective amount of the interstitial EMR-drug composition or the EMR-drug conjugate is an amount such that wound healing occurs faster for wounds treated with the interstitial EMR-drug composition or the EMR-drug conjugate than occurs for a control, e.g., an untreated wound or a wound treated with the EMR not conjugated to the drug. A patient in need thereof includes e.g., a mammal having a wound as described herein. The mammal may be, e.g., a primate, e.g., a human or a monkey, a horse, a cow, a pig, a dog, a cat, or a mouse.

Another embodiment of the invention is a method of treating wounds in a subject in need thereof by applying an effective amount of an interstitial EMR-drug composition or an EMR-drug conjugate of this invention to the wound, wherein the effective amount of the interstitial EMR-drug composition or the EMR-drug conjugate is an amount such that wound healing occurs faster in wounds treated with the interstitial EMR-drug composition or the EMR-drug conjugate, than occurs in a control, e.g. an untreated wound or a wound treated with the EMR not conjugated to the drug.

Another embodiment of the invention is a method of treating wounds by applying an effective amount of the interstitial EMR-drug composition or the EMR-drug conjugate s of this invention to the wound, wherein wound healing occurs within between 1 day and 100 days after applying the interstitial EMR-drug composition or the EMR-drug conjugate s to the wound, or between 1 day and 10 days after applying the interstitial EMR-drug composition or the EMR-drug conjugate s to the wound.

Another embodiment of the invention is a method of treating wounds by applying an effective amount of the interstitial EMR-drug composition or the EMR-drug conjugate of this invention to the wound, wherein the interstitial EMR-drug composition or the EMR-drug conjugate is applied twice daily, once daily, twice weekly, once weekly, twice monthly, or once monthly.

Another embodiment of the invention is a method of treating wounds by applying an effective amount of the interstitial EMR-drug composition or the EMR-drug conjugate of this invention to the wound, wherein the wounds are acute wounds or chronic wounds.

Another embodiment of the invention is a method of treating wounds by applying an effective amount of the interstitial EMR-drug composition or the EMR-drug conjugate of this invention to the wound, wherein the wounds are excision wounds or burn wounds.

Another embodiment of the invention is a method of treating wounds by applying an effective amount of the EMR-cell composition of this invention to the wound, wherein the wounds are chronic excisional wounds, acute excisional wounds, or burn excisional wounds.

Another embodiment of the invention is a method of treating wounds by applying an effective amount of the interstitial EMR-drug composition or the EMR-drug conjugate of this invention to the wound, wherein the wounds are diabetic ulcers or pressure wounds.

Another embodiment of the invention is a method of treating wounds by applying an effective amount of the interstitial EMR-drug composition or the EMR-drug conjugate of this invention to the wound, wherein the interstitial EMR-drug composition or the EMR-drug conjugate is applied to the wound and subsequently degraded by endogenous enzyme activity in the wound bed as healing proceeds.

Another embodiment of the invention is a method of delivering a drug to a wound in a subject in need thereof, comprising applying an effective amount of the interstitial EMR-drug composition or the EMR-drug conjugate of this invention to the wound. The effective amount of the interstitial EMR-drug composition or the EMR-drug conjugate of this invention is an amount such that wound healing occurs faster in wounds treated with the interstitial EMR-drug composition or the EMR-drug conjugate, than occurs in a control, e.g. an untreated wound or a wound treated with the EMR not conjugated to the drug.

Another embodiment of the invention is a method of prolonging delivery of a drug to a wound in a subject in need thereof, comprising applying an effective amount of the interstitial EMR-drug composition or the EMR-drug conjugate of this invention to the wound, wherein the interstitial EMR-drug composition or the EMR-drug conjugate is applied twice daily, once daily, twice weekly, once weekly, twice monthly, or once monthly, and wherein delivery of the drug occurs over at least 12 hours, at least 24 hours, at least 7 days, at least 15 days, or at least 30 days.

In the methods of this invention the interstitial EMR-drug composition or the EMR-drug conjugate of this invention may be applied to the wound in the form of a pharmaceutical composition comprising the interstitial EMR-drug composition or the EMR-drug conjugate and a pharmaceutically acceptable excipient.

### EMRs Comprising Fluorescent Labels

Another embodiment of the invention is an EMR combined with a fluorescent label. The EMRs comprising a fluorescent label as described herein may be applied to a wound (e.g., a chronic wound, such as a diabetic ulcer).

In another embodiment of the invention, an EMR is combined with a fluorescent label to indirectly monitor the progression of wound healing. The EMR comprising the fluorescent label is applied to the wound, e.g., a chronic wound, such as a diabetic ulcer, and the level of fluorescence is measured over time. By measuring the level of fluorescence of the EMR and the surrounding wound environment over time one measures indirectly the progress of wound healing as the EMR degrades and the fluorescent label is released. One may also assess the release of a drug from an EMR-drug conjugate or interstitial EMR-drug composition by combining such EMRs with an EMR-fluorescent label conjugate, applying the EMRs to the wound, and measuring a change in fluorescence in the EMR-fluorescent label conjugate or the surrounding wound environment, which change reflects a degradation of the EMR and the associated release of the drug into the wound.

In another embodiment of the invention, an EMR is combined with fluorescent labels that facilitate assessment of degradation rate or products of the EMR. The method comprises combining an EMR with a fluorescent label and then measuring the level of fluorescence in the EMR, and/or the surrounding environment over time. Preferably the fluorescent label is conjugated to a polymerizable entity of the EMR. As the EMR is degraded the fluorescent label is released and the fluorescence in the EMR and/or in the surrounding environment changes. Those of skill in the art appreciate that depending on the fluorescent label incorporated into the EMR, the intensity or amount of the fluorescence will decrease or be quenched when it is released from the EMR or the intensity or amount of fluorescence will increase once it is released from the EMR or the fluorescence wavelength will change when it is released from the EMR. The fluorescent label may be conjugated to a polymerizable entity or to an acrylate-comprising compound as described herein or by any other suitable conjugation method. The fluorescent label may also be trapped in the interstices of the polymers of the cured EMR.

In an embodiment of this invention, the fluorescent labels are selected from dyes that have emission wavelengths in the range of 350 to 2500 nm. In a preferred embodiment, the fluorescent labels are selected from dyes that have emission wavelengths in the range of 350 to 780 nm. In another preferred embodiment, the fluorescent labels are selected from dyes that have emission wavelengths in the range of 350 to 650 nm. Suitable dyes include CF®350, CF®4055, CF®405M, CF®405L, CF®430, CF®440, CF®450, CF®488A, CF®514, CF®532, CF®535ST, CF®543, CF®555, CF®568, CF®570, CF®583, CF®594, CF®594ST, CF®620R, CF®633, CF®640R, CF®647, CF®660C, CF®660R, CF®680, CF®680R, near-infrared CF® dyes, CF® dyes for multi-color super-resolution microscopy, and mixtures thereof. Suitable dyes also include fluorescein, 5-fluoresceinamine, rhodamine, acridine yellow, and mixtures thereof.

In another embodiment, EMR-fluorescent label conjugates of the invention closely match the physical properties of unfunctionalized EMRs, including the swelling properties, the stiffness, the porosity, and the oxygen permeability. The properties of the EMR-fluorescent label conjugates, like the properties of unfunctionalized EMRs, are determined through conventional methods, as described above for the interstitial EMR-drug compositions or EMR-drug conjugates. For example, a suitable shear storage modulus for the EMR-fluorescent label conjugates of the invention is between about 10 Pa and about 2000 Pa, between about 10 Pa and about 1500 Pa, between about 10 Pa and about 1000 Pa, between about 10 Pa and about 500 Pa, and between about 10 Pa and about 250 Pa. Suitable oxygen permeability for the EMR-fluorescent label conjugates of the invention is full oxygen permeability under normoxic conditions.

The swelling ratios of the EMR-fluorescent label conjugates are determined via gravimetric analysis, as described above for interstitial EMR-drug compositions or EMR-drug conjugates to evaluate the capacity of the EMR-fluorescent label conjugates to absorb water (as a surrogate for wound exudate) and desorb water (as a surrogate for wound hydration).

As described above for the interstitial EMR-drug compositions or the EMR-drug conjugates, standard mechanical measurements of the elastic modulus are collected for all EMR-fluorescent label conjugates samples using a rheometer.

As described above for the interstitial EMR-drug compositions or EMR-drug conjugates, the morphology and porosity of all EMR-fluorescent label conjugates can be determined using scanning electron microscopy to provide a representative view of the EMR-fluorescent label conjugate microstructure.

The EMR-fluorescent label conjugates must be oxygen permeable to ensure wound healing and prevent anaerobic bacterial infections. The rate of oxygen transfer through hydrated EMR-fluorescent label conjugates of various thicknesses is measured continuously using oxygen sensors under both physiological and hypoxic conditions. Monitoring oxygen permeability over time provides a temporal profile of oxygen transfer through the EMR.

In all of these characterization steps, the EMR-fluorescent label conjugates of the invention are compared to unfunctionalized EMR as a control, as described above for the interstitial EMR-drug compositions or EMR-drug conjugates.

Cytotoxicity of the EMR-fluorescent label conjugates may be evaluated by culturing human fibroblasts with the EMR-fluorescent label conjugates of the invention in wells and then quantifying fibroblast viability, morphology, and proliferation in the presence of the EMR-fluorescent label conjugates of the invention.

Cell morphology, fibroblast viability, and proliferation may be analyzed by any well-known method in the art. Fibroblast proliferation may be measured using a WST assay.

As described above for the interstitial EMR-drug compositions or EMR-drug conjugates, a scratch wound assay may be used to evaluate basic fibroblast cell recruitment induced by EMR-fluorescent label conjugates.

In vitro degradation of the EMR in the conjugates of this invention is quantified to determine a suitable degradation time, such that in vivo the EMR stays intact for long enough to provide sufficient mechanical support for cells to migrate into the wound bed but degrades sufficiently to allow further cell invasion and tissue regeneration. Degradation of the EMR may be evaluated as described above for the interstitial EMR-drug compositions or EMR-drug conjugates.

This invention provides an EMR-fluorescent label conjugate of formula (XI):

W-J-Y (XI),

wherein W is a fluorescent label, J is a linker group, and Y is an EMR.

One embodiment of the invention is an EMR-fluorescent label conjugate of formula (XI) wherein the fluorescent label is at least one dye with emission wavelengths in the range of 350 to 2500 nm.

Another embodiment of the invention is an EMR-fluorescent label conjugate of formula (XI) wherein the linker group is

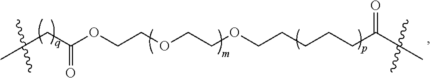

wherein q is 0 or an integer between 1 and 10, m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10.

Another embodiment of the invention is an EMR-fluorescent label conjugate of formula (XI) wherein the linker group is

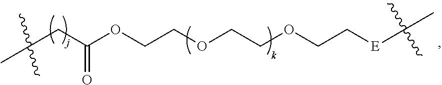

wherein E is O, N, or S, wherein j is 0 or an integer between 1 and 10, and k is 0 or an integer between 1 and 500, between 1 and 250, between 1 and 200, between 1 and 100, between 1 and 50, or between 1 and 10.

Another embodiment of the invention is an EMR-fluorescent label conjugate of formula (XI) wherein the EMR is prepared by curing a compound selected from the group consisting of unfunctionalized glucan, functionalized glucan, and mixtures thereof.

Suitable unfunctionalized glucans that may be used in the EMR-fluorescent label conjugates of the invention have a molecular weight range between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The molecular weight may be number average or weight average.

In one embodiment of the method for preparing the EMR-fluorescent label of the invention, the polymerizable entity is unfunctionalized dextran. Suitable unfunctionalized dextrans that may be used in the EMR-fluorescent label conjugates of the invention have a molecular weight range between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The molecular weight may be number average or weight average.

In one embodiment of the method for preparing the EMR-fluorescent label conjugates of the invention, the polymerizable entity is a functionalized glucan. Suitable functionalized glucans are glucans that are functionalized with polymerizable compounds, such as ethylamine, allyl carbamate, and mixtures thereof. Suitable molecular weight ranges for functionalized glucans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. In a preferred embodiment of the invention the polymerizable compound is allyl carbamate.

For use in the EMR-fluorescent label conjugates of the invention, suitable functionalized glucans have a degree of substitution between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the functionalized glucans have a degree of substitution between about 0.05 and about 0.5. In another preferred embodiment, the functionalized glucans have a degree of substitution between about 0.05 and about 0.25.

In one embodiment of the method for preparing the EMR-fluorescent label conjugates of the invention, the polymerizable entity is a functionalized dextran. Suitable functionalized dextrans are dextrans that are functionalized with polymerizable compounds, such as ethylamine, allyl carbamate, and mixtures thereof. Suitable molecular weight ranges for functionalized dextrans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. In a preferred embodiment of the invention the polymerizable compound is allyl carbamate.

For use in the EMR-fluorescent label conjugates of the invention, suitable functionalized dextrans have a degree of substitution between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the functionalized dextrans have a degree of substitution between about 0.05 and about 0.5. In another preferred embodiment, the functionalized dextrans have a degree of substitution between about 0.05 and about 0.25.

In a preferred embodiment of the EMR-fluorescent label conjugates of the invention, the polymerizable entity is dextramate. In some embodiments of the invention, the degree of substitution in the dextramate is between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the degree of substitution in dextramate is between about 0.05 and about 0.5. In another preferred embodiment, the degree of substitution in dextramate is between about 0.05 and about 0.25.

For use in the EMR-fluorescent label conjugates of the invention, suitable acrylate-comprising compounds have a molecular weight range between about 100 Da and about 250,000 Da, between about 150 Da and about 100,000 Da, between about 200 Da and about 50,000 Da, between about 250 Da and about 25,000 Da, between about 300 Da and about 10,000 Da, and between about 350 Da and about 5,000 Da. In one preferred embodiment, the acrylate-comprising compounds have a molecular weight range between about 1,000 Da and about 5,000 Da. In another preferred embodiment, the acrylate-comprising compounds have a molecular weight range between about 2,000 Da and about 4,000 Da.

One embodiment of the invention is an EMR-fluorescent label conjugates wherein the EMR component comprises a mixture of a functionalized dextran and an acrylate-comprising compound, in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

One preferred embodiment of the invention is an EMR-fluorescent label conjugates wherein the EMR component comprises a mixture of dextramate and PEGDA in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

One embodiment of the invention is an EMR-fluorescent label conjugates wherein the fluorescent label component makes up about 0.001% to about 25% of the total weight of the EMR-fluorescent label conjugate, about 0.01% to about 10% of the total weight of the EMR-fluorescent label conjugate, or about 0.1% to about 5% of the total weight of the EMR-fluorescent label conjugate.

Another embodiment of the invention is an EMR-fluorescent label conjugate of formula (XI) wherein the fluorescent label is 5-fluoresceinamine.

This invention provides a method for preparing the EMR-fluorescent label conjugate of the invention, comprising:

(a) reacting a fluorescent label of formula (XI-a):

W₁-H (XI-a)

with a structure of formula (XIIa):

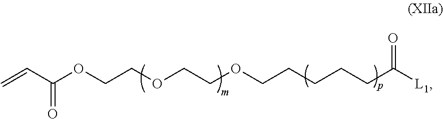

wherein L₁ is selected from the group consisting of —Cl, —Br, —I, and —ORₐ, wherein Rₐ is H, C₁-C₁₀ alkyl,

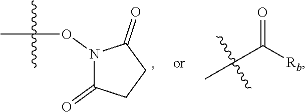

and wherein R_{b} is C₁-C₁₀ alkyl; m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10, to yield a compound of formula (XIIIa):

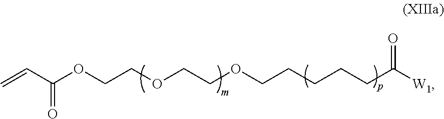

wherein W₁ is selected from the group consisting of:

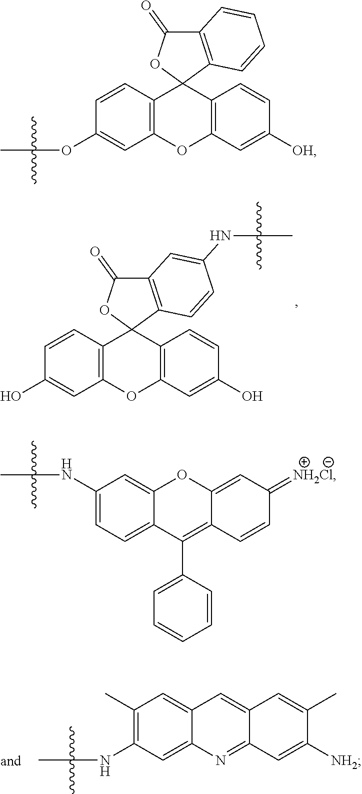

m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10;

(b) mixing the compound of formula (XIIIa) with a compound of formula (X):

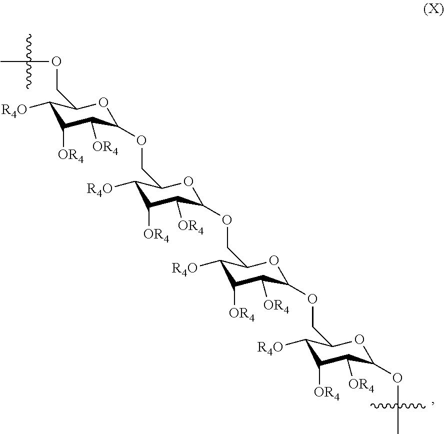

wherein R₄ is H, allyl carbamate, or mixtures thereof;

(c) optionally mixing an acrylate-comprising compound with the product of step (b); and

(d) curing the product of step (b) or step (c) with UV light and/or visible light.

Another embodiment of the invention is a method for preparing the EMR-fluorescent label conjugate of the invention, comprising:

(a) reacting a fluorescent label of formula (XI-a):

W₁-H (XI-a)

with a structure of formula (XVI):

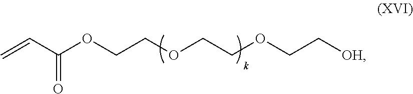

wherein k is 0 or an integer between 1 and 500, between 1 and 250, between 1 and 200, between 1 and 100, between 1 and 50, or between 1 and 10,

to yield a compound of formula (XVII):

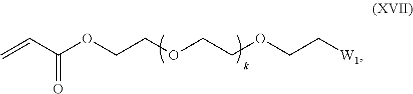

wherein W₁ is selected from the group consisting of:

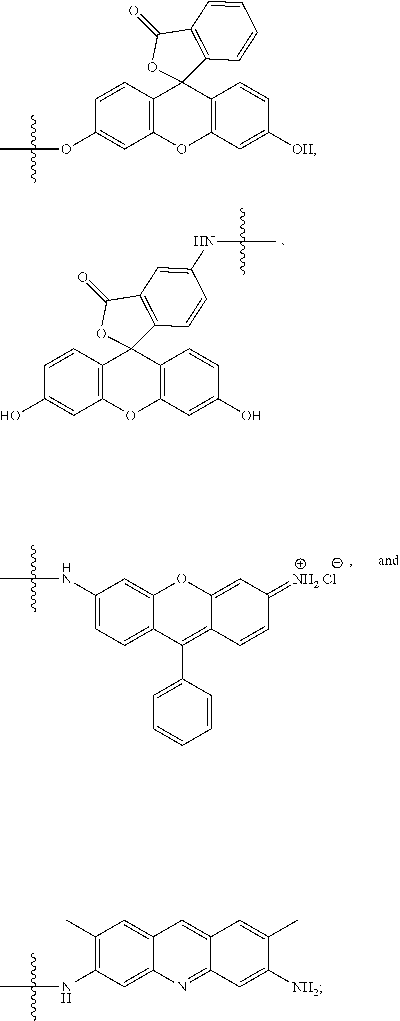

and

wherein k is 0 or an integer between 1 and 500, between 1 and 250, between 1 and 200, between 1 and 100, between 1 and 50, or between 1 and 10;

(b) mixing the compound of formula (XVII) with a compound of formula (X):

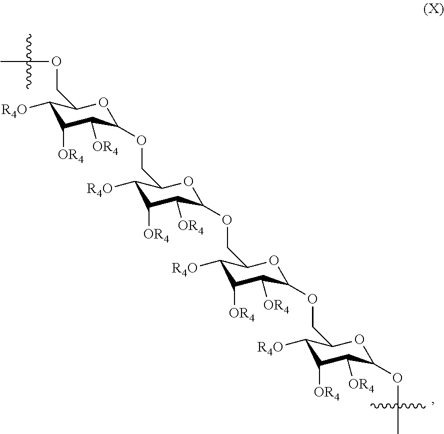

wherein R₄ is H, allyl carbamate, or mixtures thereof;

(c) optionally mixing an acrylate-comprising compound with the product of step (b); and

(d) curing the product of step (b) or step (c) with UV light and/or visible light.

In another embodiment, the EMR-fluorescent label conjugate of the invention is prepared by a method comprising the steps of:

(a) mixing a compound of formula (X):

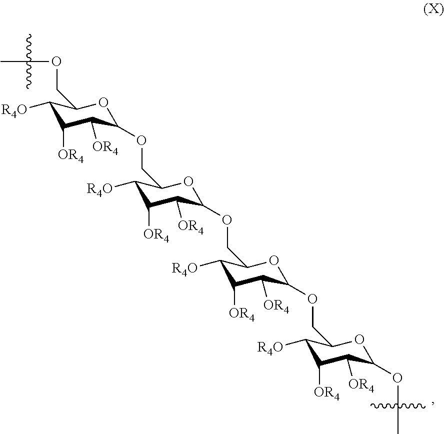

wherein R₄ is H, allyl carbamate, or mixtures thereof,

with an acrylate-comprising compound selected from the group consisting of polyethylene(glycol)diacrylate, acrylate-polyethylene(glycol)-succinimidyl valeric acid, polyethylene(glycol)acrylate, and mixtures thereof;

(b) curing the product of step (a) with UV light and/or visible light; and

(c) reacting the product of step (b) with a fluorescent label of formula (XI-a):

W₁-H (XI-a),

wherein W₁ is selected from the group consisting of:

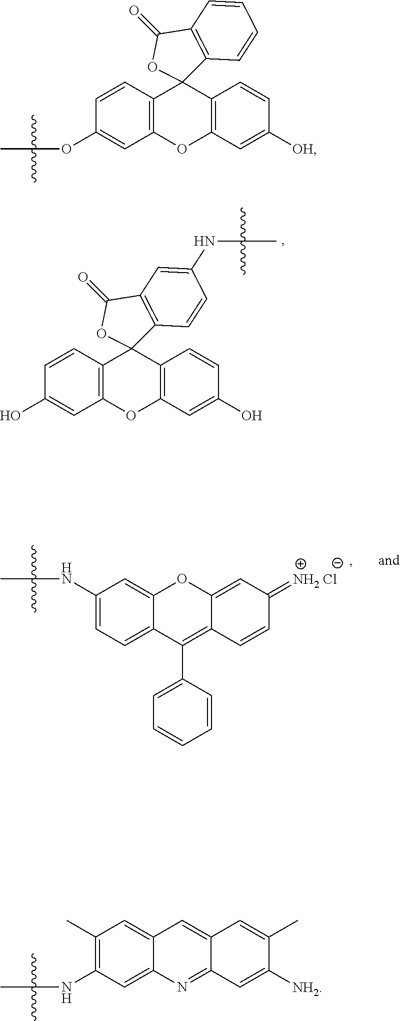

In another embodiment, the EMR-fluorescent label conjugate of the invention is prepared by a method comprising the steps of:

(a) mixing a compound of formula (X):

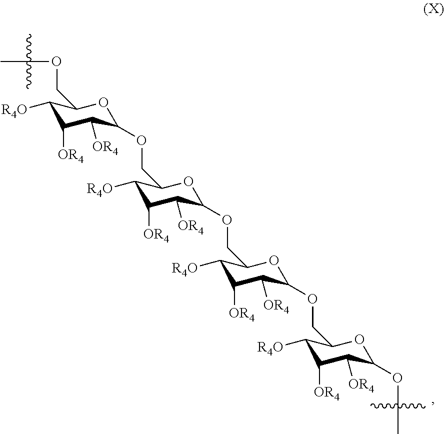

wherein R₄ is H, allyl carbamate, or mixtures thereof,

with a compound selected from the group consisting of polyethylene(glycol)diacrylate, acrylate-polyethylene(glycol)-succinimidyl valeric acid, polyethylene(glycol)acrylate, and mixtures thereof;

(b) reacting the product of step (a) with a fluorescent label of formula (XI-a):

W₁-H (XI-a),

wherein W₁ is selected from the group consisting of:

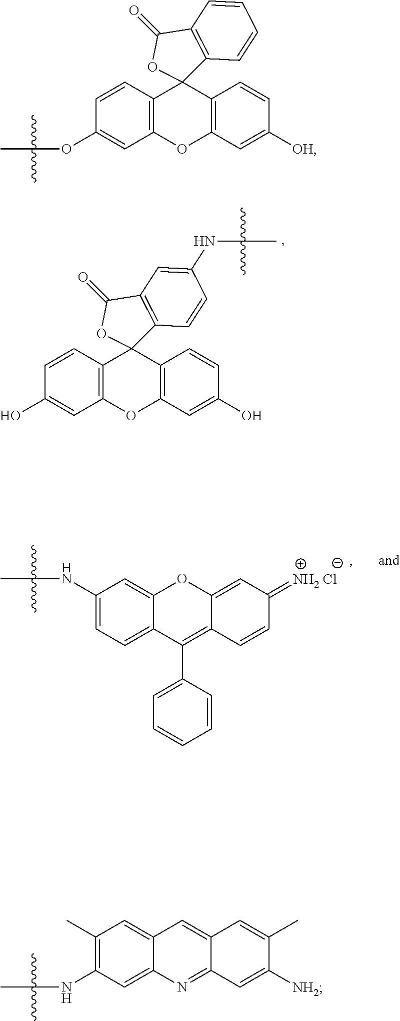

and

(c) curing the product of step (b) with UV light and/or visible light.

Suitable pore sizes for the EMR-fluorescent label conjugates of the invention are about 0.001 microns to about 100 microns, about 5 microns to about 90 microns, about 10 microns to about 80 microns, about 15 microns to about 70 microns, about 20 microns to about 60 microns, or about 25 microns to about 50 microns.

This invention provides a pharmaceutical composition comprising the EMR-fluorescent label conjugate of the invention and at least one pharmaceutically acceptable excipient.

This invention also provides a method of measuring the progress of wound treatment, comprising applying an EMR-fluorescent label conjugate to a wound and measuring healing of the wound via fluorescence microscopy and/or fluorescent spectrophotometer. Measuring may occur twice a day, once a day, twice a week, once a week, twice a month, or once a month.

This invention also provides a method of indirectly measuring the amount and/or rate of drug release from an EMR-drug conjugate, comprising applying an EMR-fluorescent label conjugate and an EMR-drug conjugate to a wound and measuring the amount and/or rate of fluorescence released from the EMR-fluorescent conjugate. The amount of fluorescence in the EMR and the surrounding wound environment may be measured via fluorescence microscopy and/or fluorescent spectrophotometer over time. The amount of fluorescence in the EMR and the surrounding wound environment is indicative of the level of EMR degradation and thus an indirect measure of drug release from the EMR-drug conjugate or EMR interstitial drug composition. Measuring may occur twice a day, once a day, twice a week, once a week, twice a month, or once a month.

This invention also provides a method of indirectly measuring the degradation products of an EMR, comprising applying an EMR-fluorescent label conjugate to a wound and measuring the amount of fluorescence in the EMR and the surrounding wound environment. The amount of fluorescence in the EMR-fluorescent composition and the surrounding wound environment is indicative of the amount of degradation products of the EMR. The amount of fluorescent label in the EMR and surrounding environment may be measured via fluorescence microscopy and/or fluorescent spectrophotometer over time. Measuring may occur twice a day, once a day, twice a week, once a week, twice a month, or once a month.

This invention also provides a method of measuring the progress of wound treatment, comprising applying an EMR-fluorescent label conjugate to a wound and measuring amount of fluorescence in the EMR and the surrounding wound environment. The amount of fluorescence in the EMR-fluorescent composition and the surrounding wound environment is indicative of the amount of healing of the wound. The amount of fluorescence in the EMR-fluorescent composition and the surrounding wound environment may be measured via fluorescence microscopy and/or fluorescent spectrophotometer. Measuring may occur twice a day, once a day, twice a week, once a week, twice a month, or once a month.

In the methods of this invention the EMR-fluorescent label conjugate of this invention may be applied to the wound in the form of a pharmaceutical composition comprising the EMR-fluorescent label conjugate and a pharmaceutically acceptable excipient.

### EMR-Cell Compositions

Another embodiment of the invention is an EMR-cell composition comprising an EMR and cells. The EMR-cell compositions of this invention may be prepared by combining, in vitro or in situ, polymerizable entities described herein for generating an EMR of this invention, with cells prior to curing and then curing the mixture such that the cells are entrapped within the EMR. The EMR-cell composition so generated may then be stored under conditions that maintain the cells viability and may be used to treat a subject in need thereof.

Examples of suitable cells that may be used in the EMR-cell composition include mammalian cells. In one embodiment, suitable cells are human cells, simian cells, porcine cells, murine cells, equine cells, bovine cells, ovine cells, feline cells, canine cells, leporine cells, or combinations thereof. In one embodiment, suitable cells are stem cells, such as mesenchymal stem cells (MSCs) (e.g., human mesenchymal stem cells (hMSCs)) which may differentiate into neurogenic, osteogenic, chondrogenic, adipogenic, tenogenic, myogenic, or stromal lineages; fibroblasts, such as primary human dermal fibroblasts (HDFs) and newborn human foreskin fibroblasts (NuFFs)); keratinocytes, such as primary human epidermal keratinocytes (HEKs); endothelial cells, such as primary endothelial cells (e.g., human umbilical vein endothelial cells (HUVECs), endothelial colony forming cells (ECFCs), primary coronary artery endothelial cells (HCAEC), primary pulmonary artery endothelial cells (HPAEC), and primary dermal microvascular endothelial cells (HDMVECs)); nerve cells; adipocytes; chondrocytes; osteocytes; myocytes; and combinations thereof. In one embodiment, the concentration of the cells in the EMR-cell composition is about at least 500 cells/mm³, about 500 to about 100,000 cells/mm³, about 5000 cell/mm³ to about 50,000 cells/mm³, about 5000 cells/mm³ to about 25,000 cells/mm³, about 7500 cells/mm³ to about 10,000 cells/mm³. In one embodiment, the concentration of the cells in the EMR-cell composition is about 1,500 cells/mm³ to about 15,000 cells/mm³.

The EMR-cell composition may comprise any of the EMRs described herein. For example, in the cured EMR-cell composition the cells are entrapped in an interstitial EMR-drug composition as described above, an EMR-drug conjugate as described above, or an EMR-fluorescent label conjugate as described above. The EMR-cell composition may be prepared by mixing the cells with a polymerizable entity selected from the group consisting of unfunctionalized glucan, functionalized glucan, and mixtures thereof. The unfunctionalized glucans may have a molecular weight range between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The molecular weight may be number average or weight average.

In another embodiment, the polymerizable entity is unfunctionalized dextran. Suitable unfunctionalized dextrans that may be used in the EMR-cell compositions of the invention have a molecular weight range between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The molecular weight may be number average or weight average.

In an embodiment of the invention, the polymerizable entity is a functionalized glucan. Suitable functionalized glucans are glucans that are functionalized with polymerizable compounds, such as ethylamine, allyl carbamate, and mixtures thereof. In a preferred embodiment of the invention, the functionalized glucan is functionalized with allyl carbamate.

Suitable molecular weight ranges for functionalized glucans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The functionalized glucans may have a degree of substitution between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the functionalized glucans have a degree of substitution between about 0.05 and about 0.5. In another preferred embodiment, the functionalized glucans have a degree of substitution between about 0.05 and about 0.25.

In an embodiment of the invention, the polymerizable entity is a functionalized dextran. Suitable functionalized dextrans are dextrans that are functionalized with polymerizable compounds, such as ethylamine, allyl carbamate, and mixtures thereof. In a preferred embodiment of the invention, the functionalized dextran is functionalized with allyl carbamate.

Suitable molecular weight ranges for functionalized dextrans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. The functionalized dextrans have a degree of substitution between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the functionalized dextrans have a degree of substitution between about 0.05 and about 0.5. In another preferred embodiment, the functionalized dextrans have a degree of substitution between about 0.05 and about 0.25.

In a preferred embodiment of the EMR-cell compositions of the invention, the polymerizable entity is dextramate. In some embodiments of the invention, the degree of substitution in the dextramate is between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the degree of substitution in dextramate is between about 0.05 and about 0.5. In another preferred embodiment, the degree of substitution in dextramate is between about 0.05 and about 0.25.

In another embodiment, the EMR-cell composition may be prepared by mixing the cells with a polymerizable entity selected from the group consisting of unfunctionalized glucan, functionalized glucan, and mixtures thereof; and with acrylate-comprising compounds and/or functionalized acrylate-comprising compounds and then curing the mixture.

Examples of suitable acrylate-comprising compound are polyethylene(glycol)diacrylate, acrylate-polyethylene(glycol)-succinimidyl valeric acid, polyethylene(glycol)acrylate, or mixtures thereof.

For use in the EMR-cell compositions of the invention, suitable acrylate-comprising compounds have a molecular weight range between about 100 Da and about 250,000 Da, between about 150 Da and about 100,000 Da, between about 200 Da and about 50,000 Da, between about 250 Da and about 25,000 Da, between about 300 Da and about 10,000 Da, and between about 350 Da and about 5,000 Da. In one preferred embodiment, the acrylate-comprising compounds have a molecular weight range between about 1,000 Da and about 5,000 Da. In another preferred embodiment, the acrylate-comprising compounds have a molecular weight range between about 2,000 Da and about 4,000 Da.

Examples of suitable functionalized acrylate-comprising compounds are acrylate-comprising compounds (e.g., polyethylene(glycol)diacrylate, acrylate-polyethylene(glycol)-succinimidyl valeric acid, polyethylene(glycol)acrylate, or mixtures thereof) that are functionalized with small molecules comprising a carboxylic acid (e.g., ARBs, antibiotics, analgesics), fluorescent labels (e.g., 5-fluorescein amine), a peptide motif, or mixtures thereof.

For use in the EMR-cell compositions of the invention, suitable functionalized acrylate-comprising compounds have a molecular weight range between about 100 Da and about 250,000 Da, between about 150 Da and about 100,000 Da, between about 200 Da and about 50,000 Da, between about 250 Da and about 25,000 Da, between about 300 Da and about 10,000 Da, and between about 350 Da and about 5,000 Da. In one preferred embodiment, the functionalized acrylate-comprising compounds have a molecular weight range between about 1,000 Da and about 5,000 Da. In another preferred embodiment, the functionalized acrylate-comprising compounds have a molecular weight range between about 2,000 Da and about 4,000 Da.

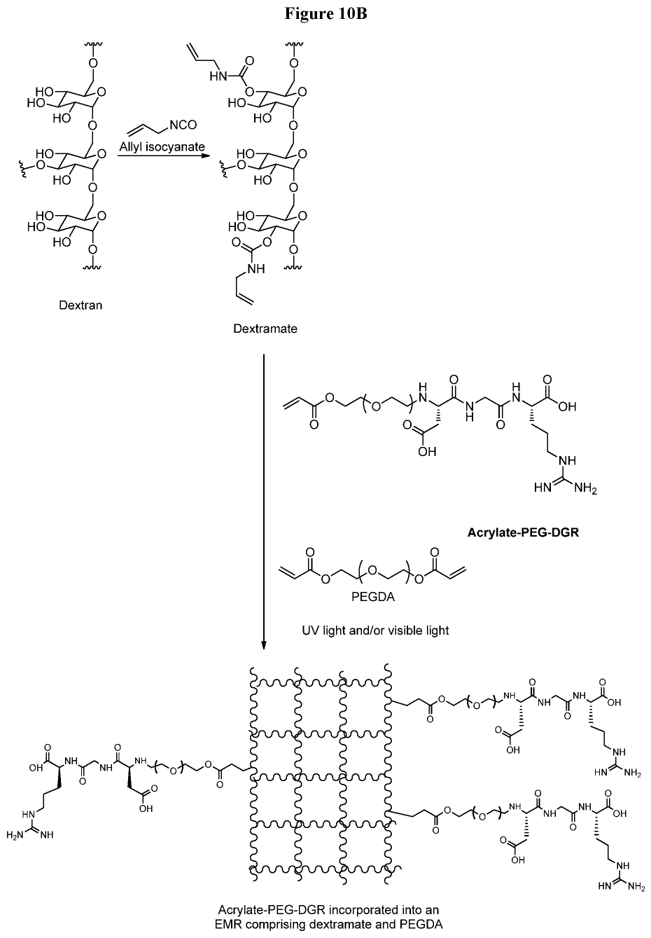

Suitable peptide motifs include peptide motifs capable of facilitating cell attachment through integrin-regulated pathways. Such peptide motifs include the arginine-glycine-aspartate tripeptide (“RGD”). In a preferred embodiment of the invention, the functionalized acrylate-comprising compound is functionalized with RGD. In a preferred embodiment of the invention, the functionalized acrylate-comprising compound is Arg-Gly-Asp-polyethylene(glycol)acrylate (RGD-PEG-acrylate). In another preferred embodiment, the functionalized acrylate-comprising compound is Asp-Gly-Arg-polyethylene(glycol)acrylate (DGR-PEG-acrylate). See FIG. 10.

In one preferred embodiment of the invention, the Arg-Gly-Asp-polyethylene(glycol)acrylate (RGD-PEG-acrylate) has a molecular weight range between about 300 Da and about 10,000 Da. In one preferred embodiment of the invention, the Arg-Gly-Asp-polyethylene(glycol)acrylate (RGD-PEG-acrylate) has a molecular weight range between about 1,000 Da and about 5,000 Da. In one preferred embodiment of the invention, the Arg-Gly-Asp-polyethylene(glycol)acrylate (RGD-PEG-acrylate) has a molecular weight range between about 2,000 Da and about 4,000 Da.

In one preferred embodiment of the invention, the Asp-Gly-Arg-polyethylene(glycol)acrylate (DGR-PEG-acrylate) has a molecular weight range between about 300 Da and about 10,000 Da. In one preferred embodiment of the invention, the Asp-Gly-Arg-polyethylene(glycol)acrylate (DGR-PEG-acrylate) has a molecular weight range between about 1,000 Da and about 5,000 Da. In one preferred embodiment of the invention, the Asp-Gly-Arg-polyethylene(glycol)acrylate (DGR-PEG-acrylate) has a molecular weight range between about 2,000 Da and about 4,000 Da.

One embodiment of the invention is an EMR-cell compositions wherein the EMR component comprises a mixture of a functionalized dextran and an acrylate-comprising compound, in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

One preferred embodiment of the invention is an EMR-cell compositions wherein the EMR component comprises a mixture of dextramate and PEGDA in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

Suitable pore sizes for the EMR in the EMR-cell compositions of the invention are about 0.001 microns to about 500 microns, about 0.01 microns to about 400 microns, about 0.1 microns to about 300 microns, about 1 micron to about 250 microns, about 2 microns to about 200 microns, about 3 microns to about 150 microns, about 4 microns to about 100 microns, about 5 microns to about 90 microns, about 10 microns to about 80 microns, about 15 microns to about 70 microns, about 20 microns to about 60 microns, or about 25 microns to about 50 microns.

In an embodiment of this invention, EMR-cell compositions of the invention closely match the physical properties of unfunctionalized EMRs, including the swelling properties, the stiffness, the porosity, and the oxygen permeability. The properties of the EMR-cell compositions, like the properties of unfunctionalized EMRs, are determined through conventional methods, as described above for the interstitial EMR-drug compositions or EMR-drug conjugates. For example, a suitable shear storage modulus for the EMR-cell compositions of the invention is between about 10 Pa and about 2000 Pa, between about 10 Pa and about 1500 Pa, between about 10 Pa and about 1000 Pa, between about 10 Pa and about 500 Pa, and between about 10 Pa and about 250 Pa. In an embodiment of an EMR-cell composition wherein the cells are bone cells, e.g., osteoclasts, osteoblasts, or osteocytes, a suitable shear storage modulus for the EMR-cell compositions of the invention is between about 2,000 Pa and about 25,000 Pa, between about 2,500 Pa and about 20,000 Pa, between about 3,000 Pa and about 15,000 Pa, between about 3,500 Pa and about 10,000 Pa, and between about 4,000 Pa and about 5,000 Pa. Preferably, the oxygen permeability for the EMR-cell compositions of the invention is full oxygen permeability under normoxic conditions.

The swelling ratios of the EMR-cell compositions are determined via gravimetric analysis, as described above for interstitial EMR-drug compositions or EMR-drug conjugates to evaluate the capacity of the EMR-cell compositions to absorb water (as a surrogate for wound exudate) and desorb water (as a surrogate for wound hydration).

As described above for the interstitial EMR-drug compositions or the EMR-drug conjugates, standard mechanical measurements of the elastic modulus are collected for all EMR-cell compositions samples using a rheometer.

As described above for the interstitial EMR-drug compositions or EMR-drug conjugates, the morphology and porosity of all EMR-cell compositions can be determined using scanning electron microscopy to provide a representative view of the EMR-cell composition microstructure.

The EMR-cell compositions must be oxygen permeable to facilitate wound healing and prevent anaerobic bacterial infections. The rate of oxygen transfer through hydrated EMR-cell compositions of various thicknesses is measured continuously using oxygen sensors under both physiological and hypoxic conditions. Monitoring oxygen permeability over time provides a temporal profile of oxygen transfer through the EMR.

In all of these characterization steps, the EMR-cell compositions of the invention are compared to unfunctionalized EMR as a control, as described above for the interstitial EMR-drug compositions or EMR-drug conjugates.

Cytotoxicity of the EMR-cell compositions and/or extractables and/or leachables may be evaluated by culturing human fibroblasts with the EMR-cell compositions of the invention in wells and then quantifying fibroblast viability, morphology, and proliferation in the presence of the EMR-cell compositions of the invention. Cytotoxicity of the EMR-cell compositions may be evaluated by incubating the EMR-cell composition in a cell culture medium, collecting the medium after the incubation (the conditioned medium) and then assaying the conditioned medium for cytotoxic effects on cells, e.g. human fibroblasts, by e.g., quantifying cell viability, morphology, and proliferation in the presence the conditioned medium.

Cell morphology, fibroblast viability, and proliferation may be analyzed by any well-known method in the art. Fibroblast proliferation may be measured using a WST assay.

As described above for the interstitial EMR-drug compositions or EMR-drug conjugates, a scratch wound assay may be used to evaluate basic fibroblast cell recruitment induced by EMR-cell compositions.

In vitro degradation of the EMR in the EMR-cell compositions of this invention is quantified to determine a suitable degradation time, such that in vivo the EMR stays intact for long enough to provide sufficient mechanical support for the cells within the composition to proliferate and permit additional cells to migrate into the wound bed but degrades sufficiently to allow further cell invasion and tissue regeneration. Degradation of the EMR may be evaluated as described above for the interstitial EMR-drug compositions or EMR-drug conjugates.

In one embodiment of the invention, the EMR-cell composition comprises a cell culture medium or a saline solution, e.g., phosphate buffered saline (PBS). In one embodiment of the invention, the EMR-cell composition is stored in a cell culture medium or a saline solution.

In one embodiment of the invention, a concentration of cells in cell culture medium is about 10,000 to about 10,000,000 cells mixed into about 25 to about 150 microliters of cell culture medium, about 30,000 to about 5,000,000 cells mixed into about 50 to about 100 microliters of cell culture medium, about 75,000 to about 2,500,000 cells mixed into about 60 to about 85 microliters of cell culture medium, or about 100,000 to about 1,000,000 cells mixed into 70 microliters of cell culture medium.

In another embodiment, the EMR-cell compositions of the invention are stored in cell culture medium. Without wishing to be bound by theory, it is contemplated that the EMR-cell compositions stored in cell culture medium may swell, causing the volume of the EMR in the EMR-cell compositions to increase. In such an embodiment, the concentration of cells per cubic millimeter may decrease as compared to EMR-cell composition immediately or shortly after curing such that the cell concentration may be, e.g., about at least 500 cells/mm³, about 500 to about 100,000 cells/mm³, about 5000 cell/mm³ to about 50,000 cells/mm³, about 5000 cells/mm³ to about 25,000 cells/mm³, about 7500 cells/mm³ to about 10,000 cells/mm³. In one embodiment, the concentration of the cells is about 1,500 cells/mm³ to about 15,000 cells/mm³.

The EMR-cell compositions of this invention may further comprise a cell culture medium. Those of skill in the art are well aware of cell culture media that are suitable for culturing different cell types. Examples of cell culture media suitable for the EMR-cell compositions of this invention include commercially available MSC media (e.g., xeno-free RB Basal-MSC media), Dulbecco's Modified Eagle Medium (DMEM), Basal Medium Eagle (BME), and Minimum Essential Medium (MEM). A preferred embodiment of the invention uses DMEM. Another preferred embodiment of the invention uses MSC media.

In another embodiment, the cell culture medium may further comprise one or more growth factors in an amount sufficient to stimulate and promote cell proliferation. Examples of suitable growth factors include epidermal growth factor (EGF); vascular endothelial growth factors (VEGF); transforming growth factors, such as transforming growth factor alpha (TGF-α) or transforming growth factor beta (TGF-β); platelet derived growth factors (PDGF); fibroblast growth factors (FGFs); keratinocyte growth factors (KGF); and neurotrophins.

The morphology, viability, and proliferation of the cells within the EMR-cell compositions of this invention may be determined by any well-known method in the art. Cell morphology may be determined by staining methods such as Eosin staining, Fuchsin staining, or another known assay in the art. Cell viability may be determined by staining methods such as red/green stains wherein cells are stained using cell-permeable dyes and living cells may be distinguished from non-living cells. or another known assay in the art. Cell proliferation may be measured using a WST assay, luminescent assay, Trypan Blue assay, or another known assay in the art. Fibroblast proliferation may be measured using a WST assay (commercially available WST-1 Cell Proliferation Assay kits are available from Cayman Chemical). Cell viability may be measured using a luminescent assay (commercially available luminescent assay kits are available from Promega). Cell viability may be measured using a Trypan Blue assay (commercially available Trypan Blue assay kits are available at Sigma Aldrich).

This invention provides a pharmaceutical composition comprising the EMR-cell composition of the invention and at least one pharmaceutically acceptable excipient.

This invention provides a method of treating wounds, comprising applying to a wound in a patient in need thereof an effective amount of the EMR-cell composition of the invention. An effective amount of the EMR-cell composition is an amount such that wound healing occurs faster for wounds treated with the EMR-cell composition than occurs for a control, e.g., an untreated wound or a wound treated solely with an EMR not combined with cells. A patient in need thereof includes e.g., a mammal having a wound as described herein. The mammal may be, e.g., a primate, e.g., a human or a monkey, a horse, a cow, a pig, a dog, a cat, or a mouse.

Another embodiment of the invention is a method of treating wounds in a subject in need thereof with an effective amount of an EMR-cell composition of this invention, wherein the effective amount of the EMR-cell composition is an amount such that wound healing occurs faster in wounds treated with the EMR-cell composition, than occurs in a control, e.g. an untreated wound or a wound treated solely with an EMR not combined with cells.

Another embodiment of the invention is a method of treating wounds by applying an effective amount of the EMR-cell composition s of this invention to the wounds, wherein wound healing occurs within between 1 day and 100 days after applying the EMR-cell compositions to the wound, or between 1 day and 10 days after applying the EMR-cell compositions to the wound.

Another embodiment of the invention is a method of treating wounds by applying an effective amount of the EMR-cell composition of this invention to the wounds, wherein the EMR-cell composition is applied twice daily, once daily, twice weekly, once weekly, twice monthly, or once monthly.

Another embodiment of the invention is a method of treating wounds by applying an effective amount of the EMR-cell composition of this invention to the wounds, wherein the wounds are acute wounds or chronic wounds.

Another embodiment of the invention is a method of treating wounds by applying an effective amount of the EMR-cell composition of this invention to the wounds, wherein the wounds are excision wounds or burn wounds.

Another embodiment of the invention is a method of treating wounds by applying an effective amount of the EMR-cell composition of this invention to the wounds, wherein the wounds are chronic excisional wounds, acute excisional wounds, or burn excisional wounds.

Another embodiment of the invention is a method of treating wounds by applying an effective amount of the EMR-cell composition of this invention to the wounds, wherein the wounds are diabetic ulcers or pressure wounds.

Another embodiment of the invention is a method of treating wounds by applying an effective amount of the EMR-cell composition of this invention to the wounds, wherein the EMR-cell composition is applied to the wound and subsequently degraded by endogenous enzyme activity in the wound bed as healing proceeds.

Another embodiment of the invention is a method of delivering cells to a wound in a subject in need thereof, comprising applying an effective amount of the EMR-cell composition of this invention. The effective amount of the EMR-cell composition of this invention is an amount such that wound healing occurs faster in wounds treated with the EMR-cell composition, than occurs in a control, e.g. an untreated wound or a wound treated solely with an EMR not combined with cells.

Another embodiment of the invention is a method of prolonging delivery of cells to a wound in a subject in need thereof, comprising applying an effective amount of the EMR-cell composition of this invention to the wound, wherein the EMR-cell composition is applied twice daily, once daily, twice weekly, once weekly, twice monthly, or once monthly, and wherein delivery of the drug occurs over at least 12 hours, at least 24 hours, at least 7 days, at least 15 days, or at least 30 days.

In the methods of this invention the EMR-cell composition of this invention may be applied to the wound in the form of a pharmaceutical composition comprising the EMR-cell composition and a pharmaceutically acceptable excipient.

Also an embodiment of this invention, is a composition comprising an -EMR-drug conjugate or interstitial EMR-drug compositions as described herein and cells incorporated into the EMR-drug conjugate or interstitial EMR-drug compositions. The drug of these composition may be any pharmaceutically active compound that promotes wound healing, prevents wound infection, and/or provides pain relief. Examples of suitable pharmaceutically active compounds for use in the interstitial EMR-drug compositions are small molecules, e.g., angiotensin receptor blockers (ARBs), antibiotics, and analgesics. Examples of suitable ARBs are valsartan, olmesartan, azilsartan, eprosartan, candesartan, telmisartan, carboxylosartan, losartan, and irbesartan. Suitable examples of antibiotics are penicillins, cephalosporins, sulfonamides, tetracyclines, aminoglycosides, glycopeptides, and macrolides. Examples of suitable analgesics are non-steroidal inflammatory drugs (e.g., aspirin, salicylic acid, ketorolac, diclofenac, indomethacin, ibuprofen, ketoprofen, and naproxen), opioids, opiates, gabapentin, and pre-gabalin. The cells of the composition may include stem cells, such as mesenchymal stem cells (MSCs) (e.g., human mesenchymal stem cells (hMSCs)) which may differentiate into neurogenic, osteogenic, chondrogenic, adipogenic, tenogenic, myogenic, or stromal lineages; fibroblasts, such as primary human dermal fibroblasts (HDFs) and newborn human foreskin fibroblasts (NuFFs)); keratinocytes, such as primary human epidermal keratinocytes (HEKs); endothelial cells, such as primary endothelial cells (e.g., human umbilical vein endothelial cells (HUVECs), endothelial colony forming cells (ECFCs), primary coronary artery endothelial cells (HCAEC), primary pulmonary artery endothelial cells (HPAEC), and primary dermal microvascular endothelial cells (HDMVECs)); nerve cells; adipocytes; chondrocytes; osteocytes; myocytes; and combinations thereof.

In an embodiment of this invention, the drug conjugated to the EMRs described herein may be any pharmaceutically active compound that comprises a carboxylic acid group. Examples of such pharmaceutically active compounds include small molecules, e.g. angiotensin receptor blockers (ARBs), antibiotics, and analgesics that comprise a carboxylic acid group. Examples of ARBs comprising a carboxylic acid group are valsartan, olmesartan, azilsartan, eprosartan, candesartan, telmisartan, carboxylosartan, and irbesartan. Examples of suitable antibiotics comprising a carboxylic acid group are penicillins and cephalosporins. Examples of analgesics comprising a carboxylic acid group are gabapentin, pre-gabalin, aspirin, salicylic acid, ketorolac, diclofenac, indomethacin, ibuprofen, ketoprofen, and naproxen. The cells of the composition may include stem cells, such as mesenchymal stem cells (MSCs) (e.g., human mesenchymal stem cells (hMSCs)) which may differentiate into neurogenic, osteogenic, chondrogenic, adipogenic, tenogenic, myogenic, or stromal lineages; fibroblasts, such as primary human dermal fibroblasts (HDFs) and newborn human foreskin fibroblasts (NuFFs)); keratinocytes, such as primary human epidermal keratinocytes (HEKs); endothelial cells, such as primary endothelial cells (e.g., human umbilical vein endothelial cells (HUVECs), endothelial colony forming cells (ECFCs), primary coronary artery endothelial cells (HCAEC), primary pulmonary artery endothelial cells (HPAEC), and primary dermal microvascular endothelial cells (HDMVECs)); nerve cells; adipocytes; chondrocytes; osteocytes; myocytes; and combinations thereof.

In an embodiment of this invention, the drug conjugated to the EMRs described herein may be any pharmaceutically active compound that comprises a functional group (e.g., an alcohol group, an ester group) that can be transformed into a carboxylic acid group. Examples of pharmaceutically active compounds that comprise a functional group that can be transformed into a carboxylic acid group are ARBs (e.g., losartan). The cells of the composition may include stem cells, such as mesenchymal stem cells (MSCs) (e.g., human mesenchymal stem cells (hMSCs)) which may differentiate into neurogenic, osteogenic, chondrogenic, adipogenic, tenogenic, myogenic, or stromal lineages; fibroblasts, such as primary human dermal fibroblasts (HDFs) and newborn human foreskin fibroblasts (NuFFs)); keratinocytes, such as primary human epidermal keratinocytes (HEKs); endothelial cells, such as primary endothelial cells (e.g., human umbilical vein endothelial cells (HUVECs), endothelial colony forming cells (ECFCs), primary coronary artery endothelial cells (HCAEC), primary pulmonary artery endothelial cells (HPAEC), and primary dermal microvascular endothelial cells (HDMVECs)); nerve cells; adipocytes; chondrocytes; osteocytes; myocytes; and combinations thereof.

In an embodiment of this invention, the EMR-cell composition may comprise a cured EMR-fluorescent label conjugate as described above. Without wishing to be bound by theory, it is contemplated that the label facilitates assessment of degradation rate or products of the EMR-cell composition, or migration of the cells into, within, or out of the composition. The fluorescent label maybe conjugated to a polymerizable entity of the EMR or to the cells. In an embodiment of this invention, a fluorescent label is selected from dyes that have emission wavelengths in the range of 350 to 2500 nm and conjugated to the cells. In a preferred embodiment, the fluorescent labels are selected from dyes that have emission wavelengths in the range of 350 to 780 nm. In another preferred embodiment, the fluorescent labels are selected from dyes that have emission wavelengths in the range of 350 to 650 nm. Suitable dyes include CF®350, CF®4055, CF®405M, CF®405L, CF®430, CF®440, CF®450, CF®488A, CF®514, CF®532, CF®535ST, CF®543, CF®555, CF®568, CF®570, CF®583, CF®594, CF®594ST, CF®620R, CF®633, CF®640R, CF®647, CF®660C, CF®660R, CF®680, CF®680R, near-infrared CF® dyes, CF® dyes for multi-color super-resolution microscopy, and mixtures thereof. Suitable dyes also include fluorescein, 5-fluoresceinamine, rhodamine, acridine yellow, and mixtures thereof. The cells of the composition may include stem cells, such as mesenchymal stem cells (MSCs) (e.g., human mesenchymal stem cells (hMSCs)) which may differentiate into neurogenic, osteogenic, chondrogenic, adipogenic, tenogenic, myogenic, or stromal lineages; fibroblasts, such as primary human dermal fibroblasts (HDFs) and newborn human foreskin fibroblasts (NuFFs)); keratinocytes, such as primary human epidermal keratinocytes (HEKs); endothelial cells, such as primary endothelial cells (e.g., human umbilical vein endothelial cells (HUVECs), endothelial colony forming cells (ECFCs), primary coronary artery endothelial cells (HCAEC), primary pulmonary artery endothelial cells (HPAEC), and primary dermal microvascular endothelial cells (HDMVECs)); nerve cells; adipocytes; chondrocytes; osteocytes; myocytes; and combinations thereof.

In another embodiment of the invention, a fluorescent label is conjugated to mitochondrial membranes to indicate mitochondrial health. Without wishing to be bound by theory, it is contemplated that active mitochondria in healthy cells maintain a mitochondrial membrane potential, and TMRM is brightly fluorescent in these mitochondria. Examples of suitable fluorescent labels include tetramethylrhodamines, such as tetramethylrhodamines methyl ester (TMRM).

In an embodiment of this invention, the EMR-cell composition may comprise cells comprising a fluorescent protein. Without wishing to be bound by theory, it is contemplated that the fluorescent protein in the cells facilitates assessment of degradation rate or products of the EMR-cell composition, or migration of the cells into, within, or out of the composition. In an embodiment of this invention, the cells expressing the fluorescent protein are transgenic cells, transfected with a nucleic acid molecule encoding the fluorescent protein. Suitable examples of fluorescent proteins include green fluorescent proteins (GFPs), yellow fluorescent proteins (YFPs), cyan fluorescent proteins (CFPs), blue fluorescent proteins (BFPs), orange fluorescent proteins (OFPs), red fluorescent proteins (RFPs), and combinations thereof.

Suitable examples of green fluorescent proteins include Emerald, Superfolder GFP, Azami Green, mWasabi, TagGFP, TurboGFP, AcGFP, ZsGreen, T-Sapphire, and proteins which have an emission maximum of between 505 nm and 511 nm.

Suitable examples of blue fluorescent proteins include EBFP, EBFP2, Azurite, mTagBFP, and proteins which have an emission maximum of between 445 nm and 456 nm.

Suitable examples of cyan fluorescent proteins include ECFP, mECFP, Cerulean, mTurquoise, CyPet, AmCyanl, Midori-Ishi Cyan, TagCFP, mTFP1, and proteins which have an emission maximum of between 474 nm and 495 nm.

Suitable examples of yellow fluorescent proteins include EYFP, Topaz, Venus, mCitrine, YPet, TagYFP, PhiYFP, ZsYellowl, mBanana, and proteins which have an emission maximum of between 524 nm and about 539 nm.

Suitable examples of orange fluorescent proteins include Kusabira Orange, Kusabira Orange2, mOrange, mOrange2, dTomato, dTomato-Tandem, TagRFP, TagRFP-T, DsRed, DsRed2, DsRed-Express, mTangerine, and proteins which have an emission maximum of between 559 nm and about 586 nm.

Suitable examples of red fluorescent proteins include mRuby, mApple, mStrawberry, AsRed2, mRFP1, JRed, mCherry, HcRedl, mRaspberry, dKeima-Tandem, HcRed-Tandem, mPlum, AQ₁₄₃, and proteins which have an emission maximum of between 592 nm and 655 nm.

In one preferred embodiment, a GFP is attached to the cells in the solution. Without wishing to be bound by theory, it is contemplated that GFP converts visible light into green light and may be attached to cells to monitor cell morphology, proliferation and viability.

### Cure-In-Place Extracellular Matrix Replacements

As previously described, EMRs, EMR-drug conjugates, and EMR-fluorescent label conjugates may be generated by mixing one or more polymerizable entities and curing using UV light and/or visible light. Once the EMR, EMR-drug conjugate, or EMR-fluorescent label conjugate is generated, it is swelled in water, packaged, and sterilized. Clinicians may then apply the EMR, EMR-drug conjugate, or EMR-fluorescent label conjugate as a patch by cutting it to size and placing it directly in the wound bed.

An embodiment of this invention is a cure-in-place (CIP) EMR. These CIP-EMRs allow a wound-specific fit by filling the wound with uncured or partially cured CIP-EMRs having a high viscosity and then curing the uncured or partially cured CIP-EMRs in the filled wound with UV light and/or visible light. Treatment of chronic wounds with the CIP-EMRs improves healing, reduces scarring, and reduces wound dehiscence for a variety of wounds.

The CIP-EMRs described herein fully fill an irregularly-shaped wound to give maximum contact between the CIP-EMRs and the wound bed. Without wishing to be bound by theory, it is contemplated that the CIP-EMRs improve healing in at least two ways: (1) by mechanically stabilizing the entire wound bed; and/or (2) by stimulating cell migration and tissue regeneration as a result of the formulation having a more complete contact with the wound bed.

In an embodiment of this invention, the CIP-EMRs of the invention comprise a high-viscosity solution comprising at least one functionalized glucan (e.g., at least one functionalized dextran), and/or at least one acrylate-comprising compound, and/or at least one substituted glucan (e.g., at least one substituted dextran), and/or at least one functionalized acrylate-comprising compound.

Examples of suitable functionalized glucans are glucans functionalized with e.g., ethylamine, allyl carbamate, or mixtures thereof.

Examples of suitable acrylate-comprising compound are polyethylene(glycol)diacrylate, acrylate-polyethylene(glycol)-succinimidyl valeric acid, polyethylene(glycol)acrylate, or mixtures thereof.

Examples of suitable substituted glucans are glucans functionalized with e.g., small molecules comprising a carboxylic acid (e.g., ARBs, antibiotics, and/or analgesics) and/or functionalized with fluorescent labels (e.g., 5-fluorescein amine).

Examples of suitable functionalized acrylate-comprising compounds are acrylate-comprising compounds (e.g., polyethylene(glycol)diacrylate, acrylate-polyethylene(glycol)-succinimidyl valeric acid, polyethylene(glycol)acrylate, or mixtures thereof) that are functionalized with small molecules comprising a carboxylic acid (e.g., ARBs, antibiotics, analgesics), fluorescent labels (e.g., 5-fluorescein amine), a peptide motif, or mixtures thereof.

Suitable peptide motifs include peptide motifs capable of facilitating cell attachment through integrin-regulated pathways. Such peptide motifs include the arginine-glycine-aspartate tripeptide (“RGD”). In a preferred embodiment of the invention, the functionalized acrylate-comprising compound is functionalized with RGD. In a preferred embodiment of the invention, the functionalized acrylate-comprising compound is Arg-Gly-Asp-polyethylene(glycol)acrylate (RGD-PEG-acrylate). In another preferred embodiment, the functionalized acrylate-comprising compound is Asp-Gly-Arg-polyethylene(glycol)acrylate (DGR-PEG-acrylate). See FIG. 10.

In one preferred embodiment of the invention, the Arg-Gly-Asp-polyethylene(glycol)acrylate (RGD-PEG-acrylate) has a molecular weight range between about 300 Da and about 10,000 Da. In one preferred embodiment of the invention, the Arg-Gly-Asp-polyethylene(glycol)acrylate (RGD-PEG-acrylate) has a molecular weight range between about 1,000 Da and about 5,000 Da. In one preferred embodiment of the invention, the Arg-Gly-Asp-polyethylene(glycol)acrylate (RGD-PEG-acrylate) has a molecular weight range between about 2,000 Da and about 4,000 Da.

In one preferred embodiment of the invention, the Asp-Gly-Arg-polyethylene(glycol)acrylate (DGR-PEG-acrylate) has a molecular weight range between about 300 Da and about 10,000 Da. In one preferred embodiment of the invention, the Asp-Gly-Arg-polyethylene(glycol)acrylate (DGR-PEG-acrylate) has a molecular weight range between about 1,000 Da and about 5,000 Da. In one preferred embodiment of the invention, the Asp-Gly-Arg-polyethylene(glycol)acrylate (DGR-PEG-acrylate) has a molecular weight range between about 2,000 Da and about 4,000 Da.

For use in the CIP-EMRs of the invention, the same glucans described above for the interstitial EMR-drug compositions and EMR-drug conjugates of the invention may be used.

Suitable molecular weight ranges for functionalized glucans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. Suitable functionalized glucans have a degree of substitution between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the functionalized glucans have a degree of substitution between about 0.05 and about 0.5. In another preferred embodiment, the functionalized glucans have a degree of substitution between about 0.05 and about 0.25.

Suitable molecular weight ranges for substituted glucans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. Suitable substituted glucans have a degree of substitution between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the functionalized glucans have a degree of substitution between about 0.05 and about 0.5. In another preferred embodiment, the functionalized glucans have a degree of substitution between about 0.05 and about 0.25.

For use in the CIP-EMRs of the invention, the same dextrans described above for the interstitial EMR-drug compositions and EMR-drug conjugates of the invention may be used.

Suitable molecular weight ranges for functionalized dextrans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. Suitable functionalized dextrans have a degree of substitution between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the functionalized dextrans have a degree of substitution between about 0.05 and about 0.5. In another preferred embodiment, the functionalized dextrans have a degree of substitution between about 0.05 and about 0.25.

Suitable molecular weight ranges for substituted dextrans are between about 10,000 Da and about 500,000 Da, between about 25,000 Da and about 250,000 Da, between about 50,000 Da and about 100,000 Da, between about 55,000 Da and about 80,000 Da, and between about 60,000 Da and about 75,000 Da. Suitable substituted dextrans have a degree of substitution between about 0.001 and about 3, between about 0.002 and about 2.75, between about 0.003 and about 2.5, between about 0.004 and about 2.25, between about 0.005 and about 2, between about 0.006 and about 1.75, between about 0.007 and about 1.5, between about 0.008 and about 1.25, between about 0.009 and about 1, between about 0.01 and about 0.9, between about 0.02 and about 0.8, between about 0.05 and about 0.7, between about 0.1 and about 0.6, between about 0.15 and about 0.5. In a preferred embodiment, the substituted dextrans have a degree of substitution between about 0.05 and about 0.5. In another preferred embodiment, the substituted dextrans have a degree of substitution between about 0.05 and about 0.25.

For use in the CIP-EMRs of the invention, the same acrylate-comprising compounds described above for the interstitial EMR-drug compositions and EMR-drug conjugates of the invention may be used.

Suitable acrylate-comprising compounds have a molecular weight range between about 100 Da and about 250,000 Da, between about 150 Da and about 100,000 Da, between about 200 Da and about 50,000 Da, between about 250 Da and about 25,000 Da, between about 300 Da and about 10,000 Da, and between about 350 Da and about 5,000 Da. In one preferred embodiment, the acrylate-comprising compounds have a molecular weight range between about 1,000 Da and about 5,000 Da. In another preferred embodiment, the acrylate-comprising compounds have a molecular weight range between about 2,000 Da and about 4,000 Da.

Suitable functionalized acrylate-comprising compounds have a molecular weight range between about 100 Da and about 250,000 Da, between about 150 Da and about 100,000 Da, between about 200 Da and about 50,000 Da, between about 250 Da and about 25,000 Da, between about 300 Da and about 10,000 Da, and between about 350 Da and about 5,000 Da. In one preferred embodiment, the functionalized acrylate-comprising compounds have a molecular weight range between about 1,000 Da and about 5,000 Da. In another preferred embodiment, the functionalized acrylate-comprising compounds have a molecular weight range between about 2,000 Da and about 4,000 Da.

A preferred embodiment of the CIP-EMRs of the invention comprises a high-viscosity solution comprising a functionalized dextran and an acrylate-comprising compound, in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

Another preferred embodiment of the CIP-EMRs of the invention comprises a high-viscosity solution comprising a mixture of dextramate and PEGDA in a ratio of 1:99 (w/w) to 99:1 (w/w), 10:90 (w/w) to 90:10 (w/w), 20:80 (w/w) to 80:20 (w/w), or 30:70 (w/w) to 70:30 (w/w).

In another embodiment of this invention, the CIP-EMRs of the invention further comprise one or more UV-crosslinking catalysts. Examples of UV-crosslinking catalysts include Irgacure catalysts (e.g., Irgacure 2959) and lithium phenyl-2,4,6-trimethylbenzoylphosphinate (LAP).

In a preferred embodiment of this invention, LAP is included in a concentration between about 0.1 to about 2.5% (w/w) in the solution. A higher concentration of LAP reduces curing time to between 1 and 5 minutes. Without wishing to be bound by theory, it is contemplated that a higher concentration of a UV-crosslinking catalyst such as LAP will result in lower curing time.

In another embodiment of this invention, the CIP-EMRs of the invention further comprise cross-linking catalysts that catalyze curing in the visible spectrum (i.e., in wavelengths ranging from 390 to 700 nm). Examples of visible-light cross-linking catalysts include eosin-Y.

In another embodiment of this invention, the CIP-EMRs of the invention further comprise cells. Examples of suitable cells that may be used in the CIP-EMRs of the invention include mammalian cells. In one embodiment, suitable cells are human cells, simian cells, porcine cells, murine cells, equine cells, bovine cells, ovine cells, feline cells, canine cells, leporine cells, or combinations thereof. In one embodiment, suitable cells are stem cells, such as mesenchymal stem cells (MSCs) (e.g., human mesenchymal stem cells (hMSCs)) which may differentiate into neurogenic, osteogenic, chondrogenic, adipogenic, tenogenic, myogenic, or stromal lineages; fibroblasts, such as primary human dermal fibroblasts (HDFs) and newborn human foreskin fibroblasts (NuFFs)); keratinocytes, such as primary human epidermal keratinocytes (HEKs); endothelial cells, such as primary endothelial cells (e.g., human umbilical vein endothelial cells (HUVECs), endothelial colony forming cells (ECFCs), primary coronary artery endothelial cells (HCAEC), primary pulmonary artery endothelial cells (HPAEC), and primary dermal microvascular endothelial cells (HDMVECs)); nerve cells; adipocytes; chondrocytes; osteocytes; myocytes; and combinations thereof.

The invention provides methods for preparing CIP-EMRs, comprising the steps of preparing a low-viscosity solution comprising at least one functionalized dextran and converting the low-viscosity solution into a high-viscosity solution. The low-viscosity solution may further comprise at least one acrylate-comprising compound and/or a UV-crosslinking catalyst and/or a visible light-crosslinking catalyst. The low-viscosity solution may further comprise cells. Suitable cells that may be used in the low-viscosity solution include mammalian cells. In one embodiment, suitable cells are human cells, simian cells, porcine cells, murine cells, equine cells, bovine cells, ovine cells, feline cells, canine cells, leporine cells, or combinations thereof. In one embodiment, suitable cells are stem cells, such as mesenchymal stem cells, such as mesenchymal stem cells (MSCs) (e.g., human mesenchymal stem cells (hMSCs)) which may differentiate into neurogenic, osteogenic, chondrogenic, adipogenic, tenogenic, myogenic, or stromal lineages; fibroblasts, such as primary human dermal fibroblasts (HDFs) and newborn human foreskin fibroblasts (NuFFs)); keratinocytes, such as primary human epidermal keratinocytes (HEKs); endothelial cells, such as primary endothelial cells (e.g., human umbilical vein endothelial cells (HUVECs), endothelial colony forming cells (ECFCs), primary coronary artery endothelial cells (HCAEC), primary pulmonary artery endothelial cells (HPAEC), and primary dermal microvascular endothelial cells (HDMVECs)); nerve cells; adipocytes; chondrocytes; osteocytes; myocytes; and combinations thereof. In one embodiment, the concentration of cells in the low-viscosity solution is about 1×10² cells/μl to about 1×10⁷ cells/μl. In another embodiment, the concentration of cells in the low-viscosity solution is about 1×10³ cells/μl to about 1×10⁶ cells/μl. In another embodiment, the concentration of cells in the low-viscosity solution is about 1×10⁴ cells/μl to about 1×10⁵ cells/μl.

The low-viscosity solution may further comprise cell culture medium. In one embodiment of the invention, at least one functionalized dextran is mixed with a cell culture medium comprising about 1×10² cells/μl to about 1×10⁷ cells/about 1×10³ cells/μl to about 1×10⁶ cells/μl. In another embodiment, the concentration of cells in the high-viscosity solution is about 1×10⁴ cells/μl to about 1×10⁵ cells/μl or about 1×10³ cells/μl to about 1×10⁶ cells/μl. In another embodiment, the concentration of cells in the high-viscosity solution is about 1×10⁴ cells/μl to about 1×10⁵ cells/μl.

In an embodiment, the low-viscosity solution comprises a polymerizable entity, e.g., a functionalized dextran and/or substituted dextran, in about 25 to about 150 microliters of cell culture medium containing about 10,000 to about 10,000,000 cells, or about 50 to about 100 microliters of cell culture medium containing about 30,000 to about 5,000,000 cells, or are mixed into about 60 to about 85 microliters of cell culture medium containing about 75,000 to about 2,500,000 cells, or about 70 microliters of any suitable cell culture medium containing about 100,000-1,000,000.

Examples of high-viscosity solutions include solutions comprising at least 0.1% (w/w) functionalized dextran and/or substituted dextran; at least 0.5% (w/w) functionalized dextran and/or substituted dextran; at least 1% (w/w) functionalized dextran and/or substituted dextran; at least 5% (w/w) functionalized dextran and/or substituted dextran; at least 10% (w/w) functionalized dextran and/or substituted dextran; at least 20% (w/w) functionalized dextran and/or substituted dextran, at least 30% (w/w) functionalized dextran and/or substituted dextran, at least 40% (w/w) functionalized dextran and/or substituted dextran, and at least 50% (w/w) functionalized dextran and/or substituted dextran. The high viscosity solutions may further comprise an acrylate-comprising compound or a UV-crosslinking catalyst or visible light-crosslinking catalyst or combinations of an acrylate-comprising compound, a UV-crosslinking catalyst and/or a visible light-crosslinking catalyst. For example, the high-viscosity solutions may further comprise at least 0.5% (w/w) acrylate-comprising compound and/or functionalized acrylate-comprising compound, at least 1% (w/w) acrylate-comprising compound and/or functionalized acrylate-comprising compound, at least 5% (w/w) acrylate-comprising compound and/or functionalized acrylate-comprising compound, at least 10% (w/w) acrylate-comprising compound and/or functionalized acrylate-comprising compound, at least 20% (w/w) acrylate-comprising compound and/or functionalized acrylate-comprising compound, at least 30% (w/w) acrylate-comprising compound and/or functionalized acrylate-comprising compound, at least 40% (w/w) acrylate-comprising compound and/or functionalized acrylate-comprising compound, and at least 50% (w/w) acrylate-comprising compound and/or functionalized acrylate-comprising compound. The high-viscosity solutions of this invention may further comprise at least 1% (w/w) UV-crosslinking catalysts and/or visible light-crosslinking catalysts, at least 2% (w/w) UV-crosslinking catalysts and/or visible light-crosslinking catalysts, at least 3% (w/w) UV-crosslinking catalysts and/or visible light-crosslinking catalysts, at least 4% (w/w) UV-crosslinking catalysts and/or visible light-crosslinking catalysts, at least 5% (w/w) UV-crosslinking catalysts and/or visible light-crosslinking catalysts, at least 6% (w/w) UV-crosslinking catalysts and/or visible light-crosslinking catalysts, at least 7% (w/w) UV-crosslinking catalysts and/or visible light-crosslinking catalysts, at least 8% (w/w) UV-crosslinking catalysts and/or visible light-crosslinking catalysts, at least 9% (w/w) UV-crosslinking catalysts and/or visible light-crosslinking catalysts, at least 10% (w/w) UV-crosslinking catalysts and/or visible light-crosslinking catalysts.

The high-viscosity solution may further comprise cells. Suitable cells that may be used in the high-viscosity solution include mammalian cells. In one embodiment, suitable cells are human cells, simian cells, porcine cells, murine cells, equine cells, bovine cells, ovine cells, feline cells, canine cells, leporine cells, or combinations thereof. In one embodiment, suitable cells are stem cells, such as mesenchymal stem cells (MSCs) (e.g., human mesenchymal stem cells (hMSCs)) which may differentiate into neurogenic, osteogenic, chondrogenic, adipogenic, tenogenic, myogenic, or stromal lineages; fibroblasts, such as primary human dermal fibroblasts (HDFs) and newborn human foreskin fibroblasts (NuFFs)); keratinocytes, such as primary human epidermal keratinocytes (HEKs); endothelial cells, such as primary endothelial cells (e.g., human umbilical vein endothelial cells (HUVECs), endothelial colony forming cells (ECFCs), primary coronary artery endothelial cells (HCAEC), primary pulmonary artery endothelial cells (HPAEC), and primary dermal microvascular endothelial cells (HDMVECs)); nerve cells; adipocytes; chondrocytes; osteocytes; myocytes; and combinations thereof. In one embodiment, the concentration of cells in the high-viscosity solution is about 1×10² cells/μl to about 1×10⁷ cells/μl. In another embodiment, the concentration of cells in the high-viscosity solution is about 1×10³ cells/μl to about 1×10⁶ cells/μl. In another embodiment, the concentration of cells in the high-viscosity solution is about 1×10⁴ cells/μl to about 1×10⁵ cells/μl.

The high viscosity solution may further comprise a cell culture medium or a saline solution, e.g., phosphate buffered saline (PBS). In one embodiment of the invention, the high-viscosity solution comprises a polymerizable entity, e.g., a functionalized dextran and/or substituted dextran in a cell culture medium comprising about 1×10² cells/μl to about 1×10⁷ cells/μl, or about 1×10³ cells/μl to about 1×10⁶ cells/μl or about 1×10⁴ cells/μl to about 1×10⁵ cells/μ.

In an embodiment, the high-viscosity solution comprises a polymerizable entity, e.g., functionalized dextran and/or substituted dextran, in about 25 to about 150 microliters of a cell culture medium containing about 10,000 to about 10,000,000 cells, or about 50 to about 100 microliters of a cell culture medium containing about 30,000 to about 5,000,000 cells, or about 60 to about 85 microliters of a cell culture medium containing about 75,000 to about 2,500,000, or about 70 microliters of any suitable cell culture medium containing about 100,000-1,000,000 cells

Examples of suitable cell culture media for use in the CIP-EMRs of the invention include commercially available MSC media (e.g., xeno-free RB Basal-MSC media), Dulbecco's Modified Eagle Medium (DMEM), Basal Medium Eagle (BME), and Minimum Essential Medium (MEM). A preferred embodiment of the invention uses MSC media. Another preferred embodiment of the invention uses DMEM.

In one embodiment of the invention, cell culture medium and cells are added to the low-viscosity solution. In one embodiment of the invention, cell culture medium and cells are added to the high-viscosity solution.

In another embodiment, a cell culture medium may further comprise one or more growth factors to stimulate and promote cell proliferation and/or migration. Growth factors for use in the CIP-EMR-cell compositions of this invention include e.g., epidermal growth factor (EGF) or vascular endothelial growth factor (VEGF; transforming growth factors, such as transforming growth factor alpha (TGF-α) or transforming growth factor beta (TGF-β); platelet derived growth factors (PDGF); fibroblast growth factors (FGFs); keratinocyte growth factors (KGF); and neurotrophins.

The morphology, viability, and proliferation of the cells in the CIP-EMRs comprising cells may be analyzed by any well-known method in the art. Cell morphology may be determined by staining methods such as Eosin staining, Fuchsin staining, or another known assay in the art. Cell viability may be determined by staining methods such as red/green stains wherein cells are stained using cell-permeable dyes and living cells may be distinguished from non-living cells. or another known assay in the art.

Examples of low-viscosity solutions include solutions comprising up to 10% (w/w) functionalized and/or substituted dextran; up to 9% (w/w) functionalized and/or substituted dextran, up to 8% (w/w) functionalized and/or substituted dextran, up to 7.5% (w/w) functionalized and/or substituted dextran, and up to 5% (w/w) functionalized and/or substituted dextran. The low-viscosity solutions may further comprise an acrylate-comprising compound or a UV-crosslinking catalysts and/or visible light-crosslinking catalysts or combinations of the an acrylate-comprising compound or a UV-crosslinking catalysts and/or visible light-crosslinking catalysts. For example, the low-viscosity solutions may include up to 10% (w/w) acrylate-comprising compound and/or functionalized acrylate-comprising compound, up to 9% (w/w) acrylate-comprising compound and/or functionalized acrylate-comprising compound, up to 8% (w/w) acrylate-comprising compound and/or functionalized acrylate-comprising compound, up to 7.5% (w/w) acrylate-comprising compound and/or functionalized acrylate-comprising compound, and up to 5% (w/w) acrylate-comprising compound and/or functionalized acrylate-comprising compound. The low-viscosity solutions may further comprise up to 2.5% (w/w) UV-crosslinking catalysts and/or visible light-crosslinking catalysts, up to 2.4% (w/w) UV-crosslinking catalysts and/or visible light-crosslinking catalysts, up to 2.3% (w/w) UV-crosslinking catalysts and/or visible light-crosslinking catalysts, up to 2.2% (w/w) UV-crosslinking catalysts and/or visible light-crosslinking catalysts, up to 2.1% (w/w) UV-crosslinking catalysts and/or visible light-crosslinking catalysts, up to 2% (w/w) UV-crosslinking catalysts and/or visible light-crosslinking catalysts, up to 1.9% (w/w) UV-crosslinking catalysts and/or visible light-crosslinking catalysts, up to 1.8% (w/w) UV-crosslinking catalysts and/or visible light-crosslinking catalysts, up to 1.7% (w/w) UV-crosslinking catalysts and/or visible light-crosslinking catalysts, up to 1.6% (w/w) UV-crosslinking catalysts and/or visible light-crosslinking catalysts, up to 1.5% (w/w) UV-crosslinking catalysts and/or visible light-crosslinking catalysts, up to 1.4% (w/w) UV-crosslinking catalysts and/or visible light-crosslinking catalysts, up to 1.3% (w/w) UV-crosslinking catalysts and/or visible light-crosslinking catalysts, up to 1.2% (w/w) UV-crosslinking catalysts and/or visible light-crosslinking catalysts, up to 1.1% (w/w) UV-crosslinking catalysts and/or visible light-crosslinking catalysts, up to 1% (w/w) UV-crosslinking catalysts and/or visible light-crosslinking catalysts, up to 0.9% (w/w) UV-crosslinking catalysts and/or visible light-crosslinking catalysts, up to 0.8% (w/w) UV-crosslinking catalysts and/or visible light-crosslinking catalysts, up to 0.75% (w/w) UV-crosslinking catalysts and/or visible light-crosslinking catalysts, and up to 0.5% (w/w) UV-crosslinking catalysts and/or visible light-crosslinking catalysts. Further examples of low-viscosity solutions include solutions comprising 8% allyl carbamate-dextran (dextramate)/2% PEGDA/0.1% Irgacure 2959 (w/w). Viscosity may be determined using a viscometer. Further examples of low-viscosity solutions include solutions with a room-temperature viscosity ranging from 0.0091 poise to 14.12 poise. Examples of high-viscosity solutions are solutions with a room-temperature viscosity higher than 14.12 poise.

In an embodiment of this invention, the low-viscosity solution has a room-temperature viscosity ranging from 0.0091 poise to 14.12 poise, from 0.0091 poise to 12 poise, from 0.0091 poise to 10 poise, from 0.0091 poise to 5 poise, from 0.0091 poise to 2 poise, from 0.0091 poise to 1 poise, from 0.0091 poise to 0.50 poise, from 0.0091 poise to 0.10 poise, from 0.0091 poise to 0.05 poise.

In an embodiment of this invention, the low-viscosity solution is aqueous.

In an embodiment of this invention, the low-viscosity solution is converted into a high-viscosity solution by increasing the concentration of at least one functionalized dextran, and/or at least one substituted dextran, and/or at least one acrylate-comprising compound, and/or at least one functionalized acrylate-comprising compound.

Suitable concentrations may be determined by known methods. For example, the water content of a low-viscosity solution may be titrated down until the solution is saturated with at least one functionalized dextran, and/or at least one substituted dextran, and/or at least one acrylate-comprising compound, and/or at least one functionalized acrylate-comprising compound. An example of high-viscosity solution comprises 5.6 g dextramate, 1.4 g PEGDA, 0.1% Irgacure, and water to bring the solution to 10 mL total.

In an embodiment of this invention, the low-viscosity solution is converted into a high-viscosity solution by adding high-viscosity, non-irritating polar solvents and/or solvent additives. Suitable solvents include, for example, glycerol (1412 cP), medical honey (10,000 cP), and isopropanol (1.96 cP). Suitable solvent additives include, for example, BYK-420 and Garamite-7305. For example, a low-viscosity solution comprising 800 mg dextramate, 200 mg PEGDA, and 0.1% Irgacure in 10 mL water may be converted into a high-viscosity solution by adding glycerol and/or medical honey and/or replacing some or all of water with glycerol and/or medical honey. An example of a high-viscosity solution comprises 800 mg dextramate, 200 mg PEGDA, and 0.1% Irgacure, and 10 mL glycerol.

In an embodiment of this invention, the low-viscosity solution is converted into a high-viscosity solution by partially curing the low-viscosity solution with UV-light and/or visible light. Conditions for partially curing the low-viscosity solutions may be determined using known methods. For example, conditions may be determined by modifying the conditions for curing known EMRs and systematically reducing the curing times and/or UV intensity and/or visible light intensity required for curing those known EMRs.

In an embodiment of this invention, solutions are considered to have a suitably high viscosity when they are capable of filling a wound bed, and/or are capable of conforming to wound boundaries, and/or when they are capable of creating a uniformly-cured EMR upon exposure to UV light and/or visible light.

This invention provides a pharmaceutical composition comprising the CIP-EMRs of the invention and at least one pharmaceutically acceptable excipient.

In an embodiment of this invention, a solution comprising at least one functionalized dextran and optionally an acrylate-comprising compound for making the CIP-EMRs of the invention may be packaged in a light-blocking container as a pre-mixed suspension.

In an embodiment of this invention, the CIP-EMRs of the invention further comprise a drug with a carboxylic acid group, e.g., an ARB, conjugated to the EMR. The ARB may be selected from the group consisting of valsartan, olmesartan, azilsartan, eprosartan, candesartan, telmisartan, carboxylosartan, irbesartan, losartan, and mixtures thereof.

In an embodiment of this invention, the CIP-EMRs comprise at least one substituted dextran that is functionalized with a small molecule, and/or at least one acrylate-comprising compound that is functionalized with a small molecule, and/or at least one acrylate-comprising compound. These small molecules in the CIP-EMRs of the invention also accelerate wound closure, prevent wound infection, and/or provide pain relief.

This invention provides substituted dextrans of formula (XXI), which are functionalized with a small molecule comprising a carboxylic acid group, that may be used in the CIP-EMRs of the invention:

Q₃-X—Y₁ (I-1),

wherein Q₃ is the residue of a small molecule comprising a carboxylic acid group selected from the group consisting of ARBs, antibiotics, analgesics, and mixtures thereof;

X is a linker group selected from the group consisting of a bond,

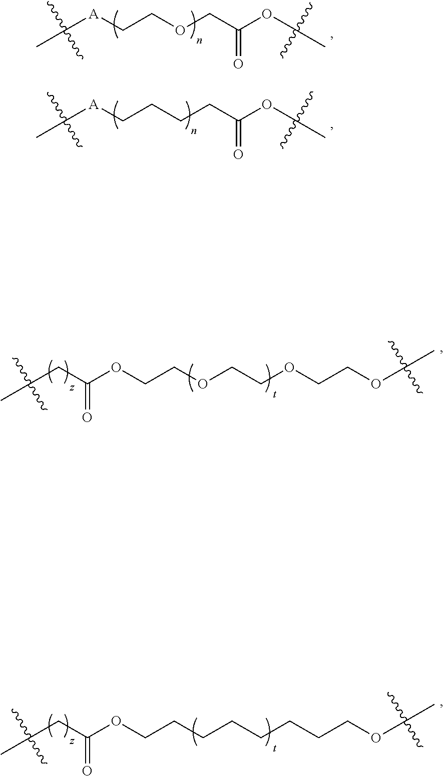

and mixtures thereof, wherein A is O, S, or NH, n is 0 or an integer between 1 and 500, between 1 and 250, between 1 and 200, between 1 and 100, between 1 and 50, or between 1 and 10; t is 0 or an integer between 1 and 500, between 1 and 250, between 1 and 200, between 1 and 100, between 1 and 50, or between 1 and 10; and z is 0 or an integer between 1 and 10; and

Y₁ is a dextran functionalized with ethylamine, allyl carbamate, or mixtures thereof.

In one embodiment, the dextrans of formula (XXI) are prepared by:

(a) reacting a small molecule of formula (XVIII) with a compound of formula (XIXa) or formula (XIXb) to yield a compound of formula (XXa) or (XXb):

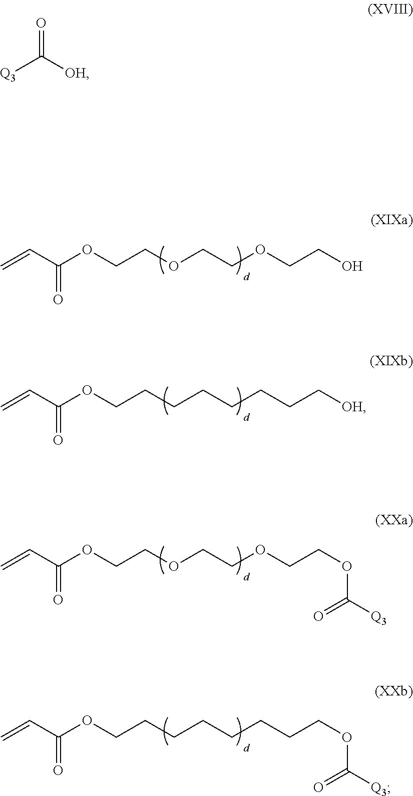

wherein Q₃ is the residue of any small molecule (e.g., a small molecule selected from the group consisting of ARBs, antibiotics, and analgesics) and wherein d is 0 or an integer between 1 and 500, between 1 and 250, between 1 and 200, between 1 and 100, between 1 and 50, or between 1 and 10;

(b) reacting the compound of formula XXa or formula XXb with acryl carbamate groups on a dextran of formula X

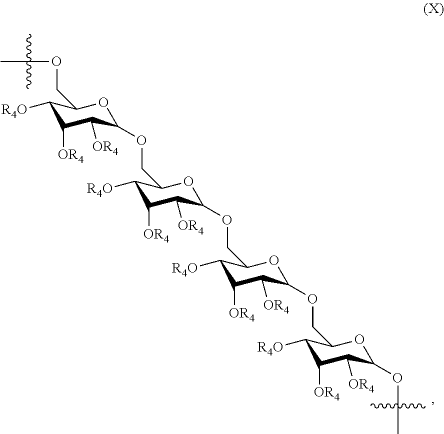

wherein R₄ is H, allyl carbamate, or mixtures thereof.

In one preferred embodiment of the method of making dextrans of formula (XXI), Q₃ is selected from the group consisting of

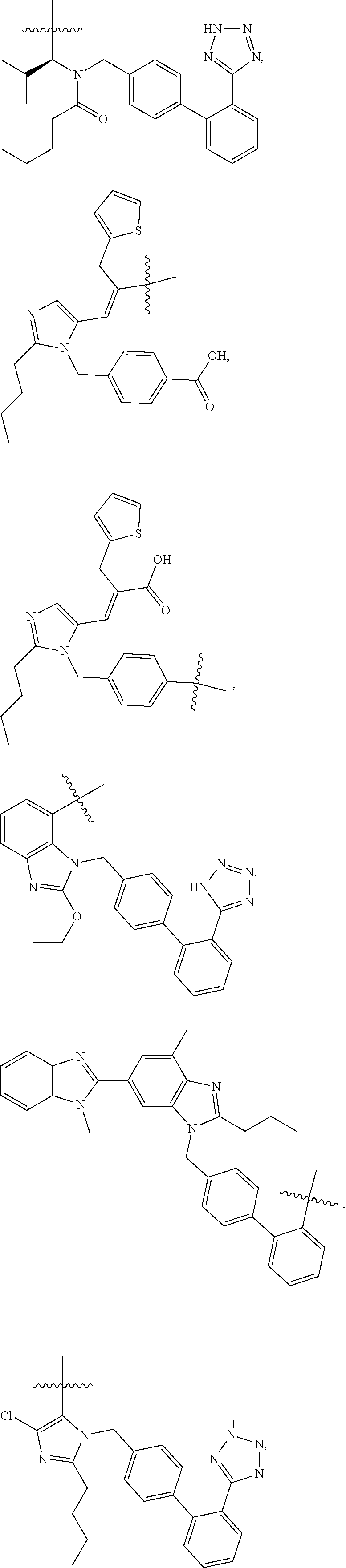

and mixtures thereof.

FIG. 3A illustrates reacting polyethylene(glycol)acrylate (10 mg) with valsartan (8.7 mg) using dicyclohexylcarbodiimide (DCC, 4.95 mg) and dimethylaminopyridine (DMAP, 2.93 mg) in dimethylformamide (DMF, 5 mL). FIG. 4A illustrates curing a mixture of the valsartan-functionalized polyethylene(glycol)acrylate resulting from the reaction of FIG. 3A, polyethylene(glycol)diacrylate, and dextramate to make an EMR-drug conjugate.

In an embodiment of this invention, the CIP-EMRs comprise at least one substituted dextran that is functionalized with an ARB and/or comprise at least one acrylate-comprising compound that is functionalized with an ARB and/or at least one acrylate-comprising compound.

In an embodiment of this invention, CIP-EMRs of the invention comprising an ARB accelerate wound closure.

This invention provides substituted dextrans of formula (I-1), which are functionalized with an ARB, that may be used in the CIP-EMRs of the invention:

Q-X—Y₁ (I-1),

wherein Q is an ARB selected from the group consisting of valsartan, olmesartan, azilsartan, eprosartan, candesartan, telmisartan, carboxylosartan, irbesartan, losartan, and mixtures thereof;

X is a linker group selected from the group consisting of a bond,

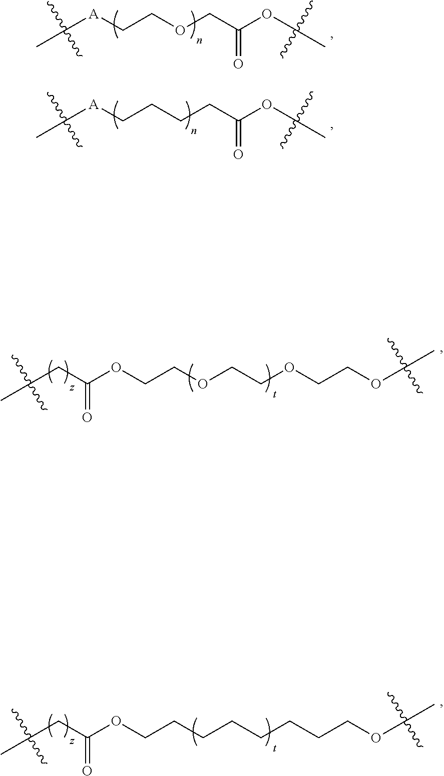

and mixtures thereof, wherein A is O, S, or NH, n is 0 or an integer between 1 and 500, between 1 and 250, between 1 and 200, between 1 and 100, between 1 and 50, or between 1 and 10; t is 0 or an integer between 1 and 500, between 1 and 250, between 1 and 200, between 1 and 100, between 1 and 50, or between 1 and 10; and z is 0 or an integer between 1 and 10; and

Y₁ is a dextran functionalized with ethylamine, allyl carbamate, or mixtures thereof.

This invention provides a method for preparing the substituted dextrans of formula (I-1), which are functionalized with an ARB, comprising:

(a) transforming the ARB with a structure of formula (IIa) into a compound of formula (IIb):

Q₁-OH (IIa)

Q₁-L (IIb),

wherein Q₁ is selected from the group consisting of:

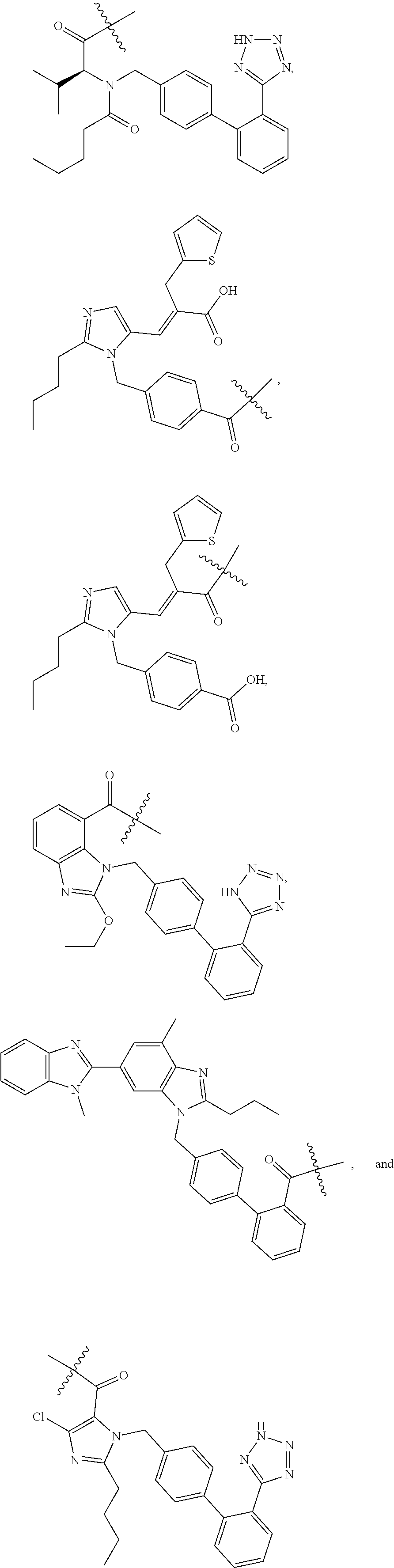

wherein L is selected from the group consisting of: —Cl, —Br, —I, and —OR₁, wherein R₁ is C₁-C₁₀ alkyl or

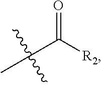

and wherein R₂ is C₁-C₁₀ alkyl;

(b) reacting the compound of formula (IIb) with a compound of formula (Ma) or formula (IIIb) to yield a compound of formula (IVa) or (IVb):

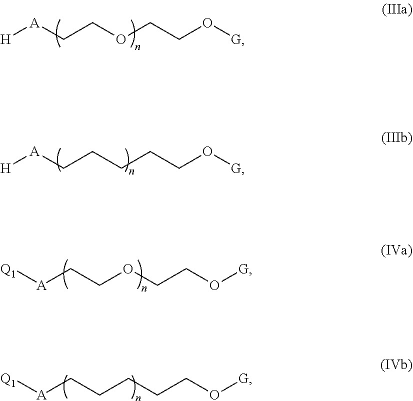

wherein G is a protecting group, A is O, S, or NH, n is 0 or an integer between 1 and 500, between 1 and 250, between 1 and 200, between 1 and 100, between 1 and 50, or between 1 and 10; and Q₁ is the same as defined in step (a);

(c) deprotecting the compound of formula (IVa) or formula (IVb) to yield a compound of formula (Va) or formula (Vb):

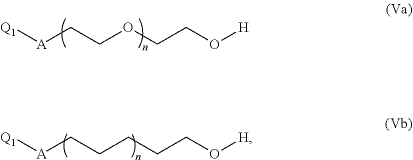

wherein Q₁ is the same as defined in step (a) and A and n are the same as defined in step (b);

(d) oxidizing the compound of formula (IVa) or formula (IVb) to yield a product of formula (VIa) or formula (VIb):

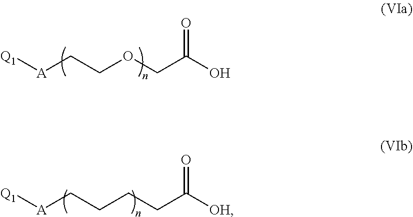

wherein Q₁ is the same as defined in step (a) and A and n are the same as defined in step (b);

(e) transforming the compound of formula (VIa) or formula (VIb) to yield a compound of formula (VIIa) or formula (VIIb):

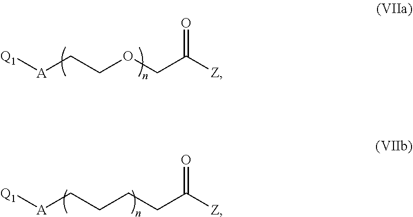

wherein Z is —Cl, —Br, —I, and —OR₃, wherein R₃ is C₁-C₁₀ alkyl, and wherein Q₁ is the same as defined in step (a) and A and n are the same as defined in step (b); and

(f) reacting the product of compound of formula (VIIa) or formula (VIIb) with free hydroxyl groups on a compound of formula (X):

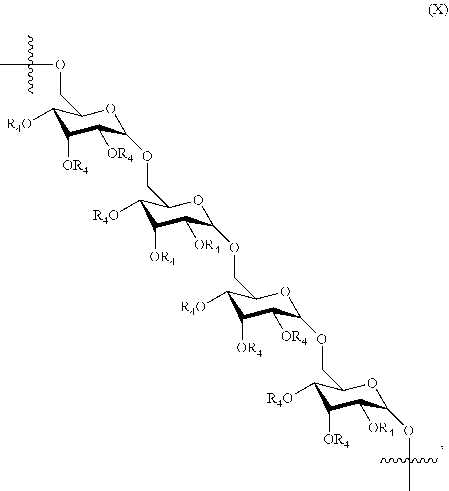

wherein R₄ is H, allyl carbamate, or mixtures thereof.

Another embodiment of the invention is a method for preparing the substituted dextrans of formula (I-1), which are functionalized with an ARB, comprising:

(a) transforming the ARB with a structure of formula (IIc) into a compound of formula (IIa-1):

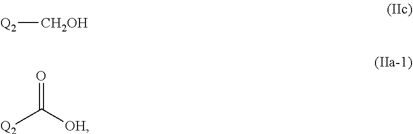

wherein Q₂ is

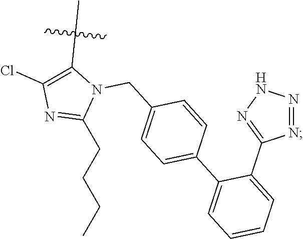 [SMILES: *c1c(Cl)nc(CCCC)n1Cc1ccc(-c2ccccc2-c2nn[nH]n2)cc1]

(b) transforming the compound formula (IIa-1) into a compound of formula (IIb-1):

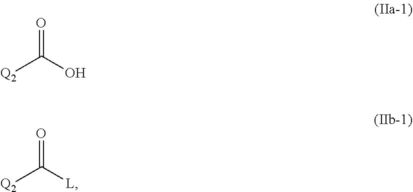

wherein Q₂ is the same as defined in step (a), wherein L is selected from the group consisting of —Cl, —Br, —I, and —OR₁, wherein R₁ is C₁-C₁₀ alkyl or

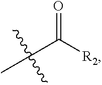

and wherein R₂ is C₁-C₁₀ alkyl;

(c) reacting the compound of formula (IIb-1) with a compound of formula (Ma) or formula (IIIb) to yield a compound of formula (IVa-1) or (IVb-1):

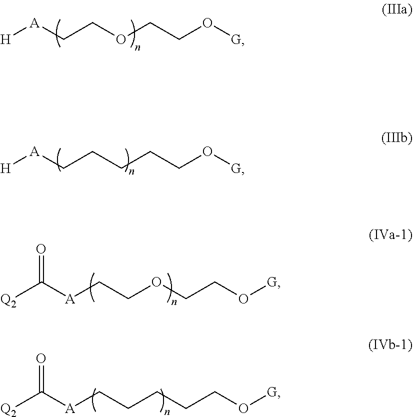

wherein G is a protecting group selected from the group consisting of C₁-C₁₀ unbranched or branched alkyl; —SiMe₃; —SiEt₃; —Si(iPr)₃; —SiPh₃; —SiMe₂iPr; —SiMe₂Et; —SiEt₂iPr; and —CH₂-Ph, wherein the Ph is unsubstituted or substituted with at least one substituent selected from the group consisting of —OMe, —NO₂, —F, —Cl, —Br, —I, —CF₃, —SiMe₃, and —CN,

A is O, S, or NH,

n is 0 or an integer between 1 and 500, between 1 and 250, between 1 and 200, between 1 and 100, between 1 and 50, or between 1 and 10; and Q₂ is the same as defined in step (a);

(d) deprotecting the compound of formula (IVa-1) or formula (IVb-1) to yield a compound of formula (Va-1) or formula (Vb-1):

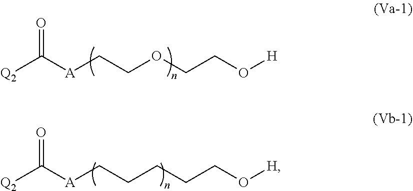

wherein Q₂ is the same as defined in step (a) and A and n are the same as defined in step (c);

(e) oxidizing the compound of formula (IVa-1) or formula (IVb-1) to yield a product of formula (VIa-1) or formula (VIb-1):

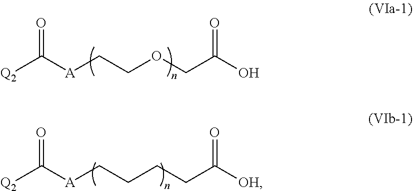

wherein Q₂ is the same as defined in step (a) and A and n are the same as defined in step (c);

(f) transforming the compound of formula (VIa-1) or formula (VIb-1) to yield a compound of formula (VIIa-1) or formula (VIIb-1):

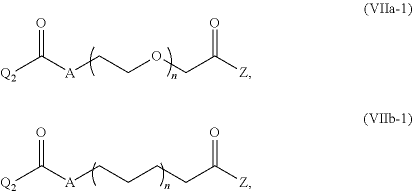

wherein Z is —Cl, —Br, —I, and —OR₃, wherein R₃ is C₁-C₁₀ alkyl, and wherein Q₂ is the same as defined in step (a) and A and n are the same as defined in step (c); and

(g) reacting the product of compound of formula (VIIa-1) or formula (VIIb-1) with free hydroxyl groups on a compound of formula (X):

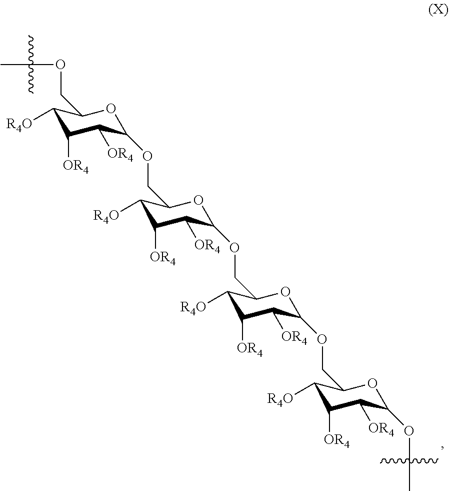

wherein R₄ is H, allyl carbamate, or mixtures thereof.

Another embodiment of the invention is a method for preparing the substituted dextrans of formula (I-1), which are functionalized with an ARB, comprising:

(a) reacting a compound of formula (IIa) with a structure of formula (XIVa) or formula (XIVb) to yield a compound of formula (XVa) or formula (XVb):

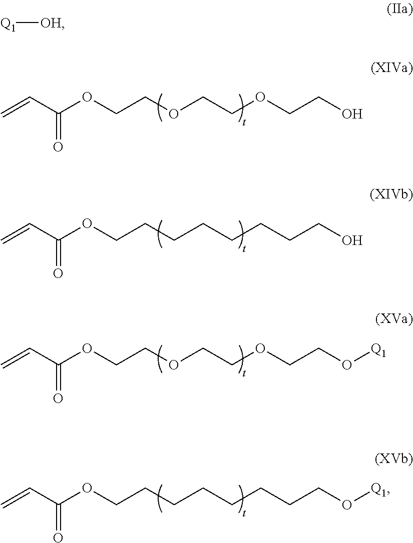

wherein t is 0 or an integer between 1 and 500, between 1 and 250, between 1 and 200, between 1 and 100, between 1 and 50, or between 1 and 10; and Q₁ is selected from the group consisting of:

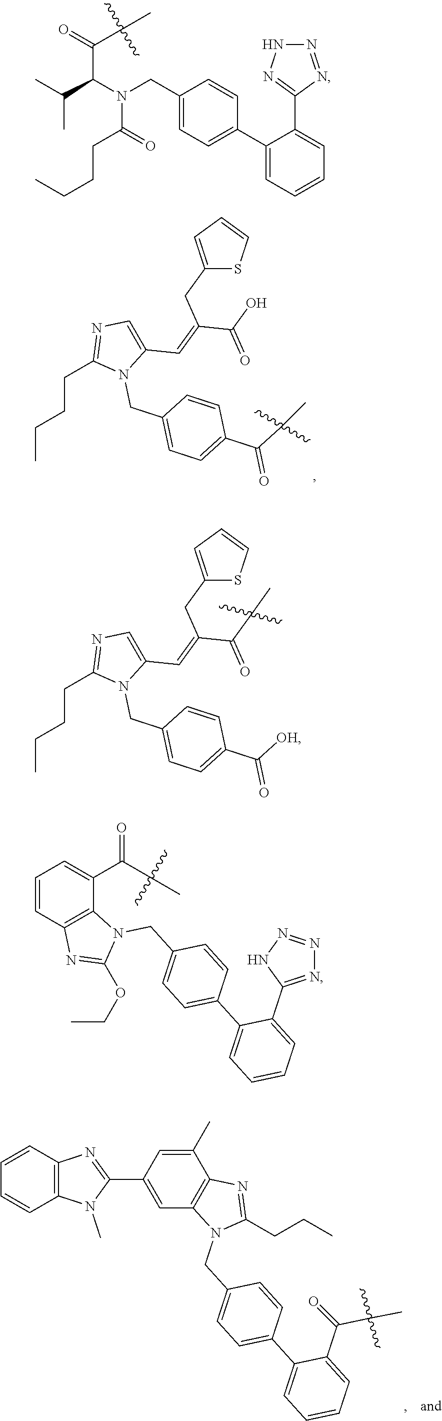 [SMILES: *C(=O)[C@H](C(C)C)N(Cc1ccc(-c2ccccc2-c2nn[nH]n2)cc1)C(=O)CCCC ; *C(=O)c1ccc(Cn2c(/C=C(\Cc3cccs3)C(=O)O)cnc2CCCC)cc1 ; *C(=O)/C(=C/c1cnc(CCCC)n1Cc1ccc(C(=O)O)cc1)Cc1cccs1 ; *C(=O)c1cccc2nc(OCC)n(Cc3ccc(-c4ccccc4-c4nnn[nH]4)cc3)c12 ; *C(=O)c1ccccc1-c1ccc(Cn2c(CCC)nc3c(C)cc(-c4nc5ccccc5n4C)cc32)cc1]

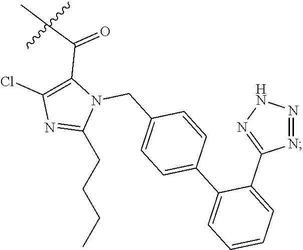 [SMILES: *C(=O)c1c(Cl)nc(CCCC)n1Cc1ccc(-c2ccccc2-c2nn[nH]n2)cc1]

and

(b) mixing the compound of formula (XVa) or formula (XVb) with a compound of formula (X):

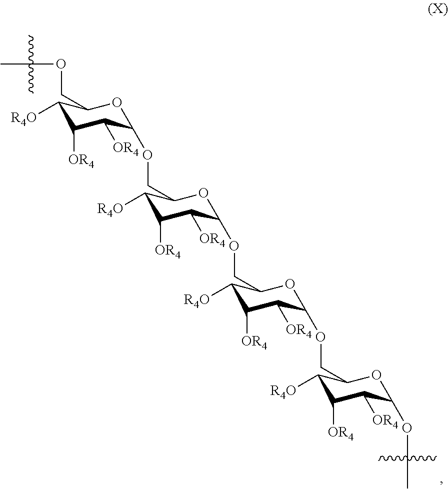

wherein R₄ is H, allyl carbamate, or mixtures thereof.

Another embodiment of the invention is a method for preparing the substituted dextrans of formula (I-1), which are functionalized with an ARB, comprising:

(a) transforming a compound of formula (XIVa) or formula (XIVb) into a compound of formula (XIVa-1) or formula (XIVb-1):

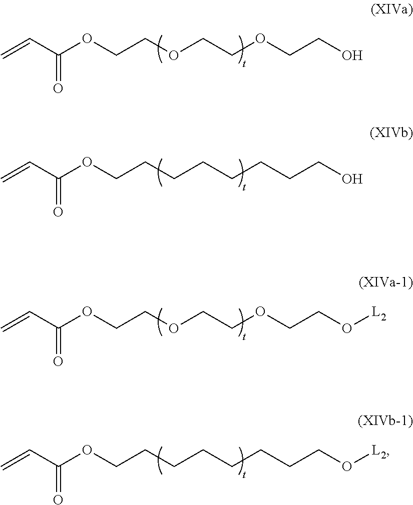

wherein t is 0 or an integer between 1 and 500, between 1 and 250, between 1 and 200, between 1 and 100, between 1 and 50, or between 1 and 10; and L₂ is a group selected from the group consisting of C₁-C₁₀ alkyl or

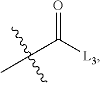

and wherein L₃ is C₁-C₁₀ alkyl;

(b) reacting the compound of formula (XIVa-1) or formula (XIVb-1) with a compound of formula (IIc):

Q₂-CH₂OH (IIc)

to yield a compound of formula (XIVa-2) or formula (XIVb-2):

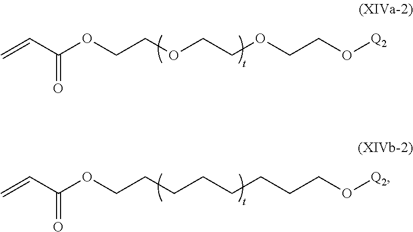

wherein Q₂ is

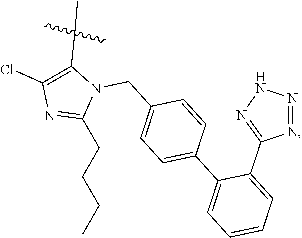 [SMILES: *c1c(Cl)nc(CCCC)n1Cc1ccc(-c2ccccc2-c2nn[nH]n2)cc1]

and wherein t and L₂ are as defined in step (a); and

(c) mixing the compound of formula (XIVa-2) or formula (XIVb-2) with a compound of formula (X):

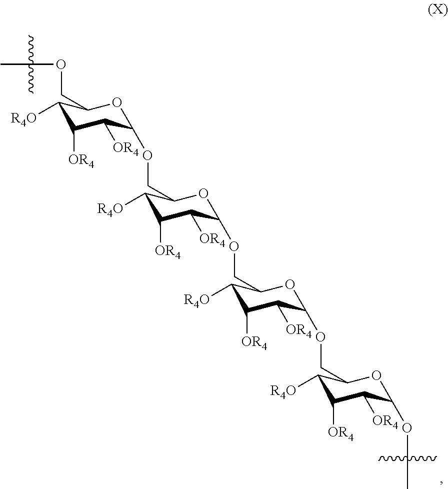

wherein R₄ is H, allyl carbamate, or mixtures thereof.

In an embodiment of this invention, the CIP-EMRs of the invention further comprise a fluorescent label selected from at least one dye with emission wavelengths in the range of 350 to 2500 nm.

In an embodiment of this invention, the CIP-EMRs of the invention are a high-viscosity solution comprising at least one substituted dextran that is functionalized with a fluorescent label, and/or at least one acrylate-comprising compound that is functionalized with a fluorescent label, and/or at least one acrylate-comprising compound.

In an embodiment of this invention, CIP-EMRs of the invention further comprising a fluorescent label facilitate measurement of wound healing progress via, for example, fluorescence microscopy studies measuring the change in fluorescence in the CIP-EMR and/or the surrounding wound environment.

This invention provides substituted dextrans of formula (I-2), which are functionalized with a fluorescent label, that may be used in the CIP-EMRs of the invention:

W-J-Y₁ (I-2),

wherein W is a fluorescent label selected from at least one dye with emission wavelengths in the range of 350 to 2500 nm;

J is

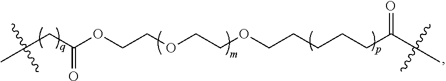

wherein q is 0 or an integer between 1 and 10, m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10; and

Y₁ is a dextran functionalized with ethylamine, allyl carbamate, or mixtures thereof.

This invention provides a method for preparing the substituted dextrans of formula (I-2), which are functionalized with a fluorescent label, comprising:

(a) reacting a fluorescent label of formula (XI-a) with a structure of formula (XIIa) to yield a compound of formula (XIIIa):

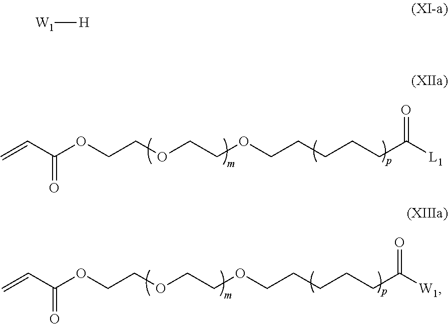

wherein m is 0 or an integer between 1 and 500, between 1 and 250, between 1 and 200, between 1 and 100, between 1 and 50, or between 1 and 10; p is 0 or an integer between 1 and 500, between 1 and 250, between 1 and 200, between 1 and 100, between 1 and 50, or between 1 and 10; L₁ is selected from the group consisting of —Cl, —Br, —I, and —ORₐ, wherein Rₐ is H, C₁-C₁₀ alkyl,

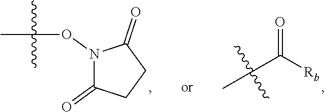

and wherein R_{b} is C₁-C₁₀ alkyl; m is 0 or an integer between 1 and 500, between 1 and 250, between 1 and 200, between 1 and 100, between 1 and 50, or between 1 and 10; and p is 0 or an integer between 1 and 500, between 1 and 250, between 1 and 200, between 1 and 100, between 1 and 50, or between 1 and 10; and W₁ is selected from the group consisting of:

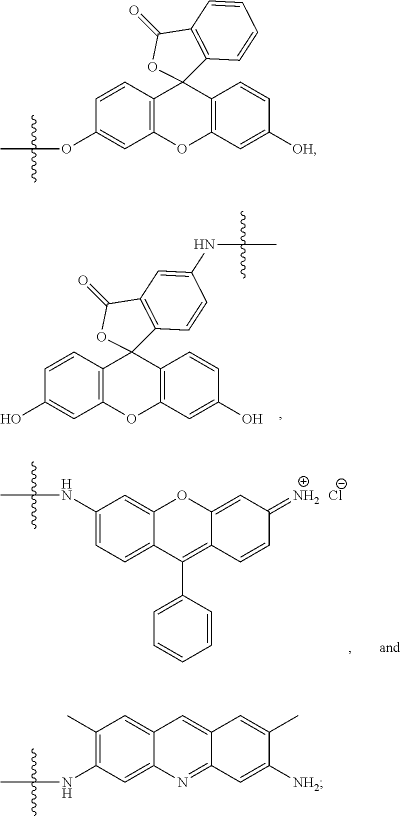 [SMILES: *Oc1ccc2c(c1)Oc1cc(O)ccc1C21OC(=O)c2ccccc21 ; *Nc1ccc2c(c1)C(=O)OC21c2ccc(O)cc2Oc2cc(O)ccc21 ; *Nc1ccc2c(-c3ccccc3)c3ccc(=[NH2+])cc-3oc2c1 ; [Cl-] ; *Nc1cc2nc3cc(N)c(C)cc3cc2cc1C]

and

(b) mixing the compound of formula (XIIIa) with a compound of formula (X):

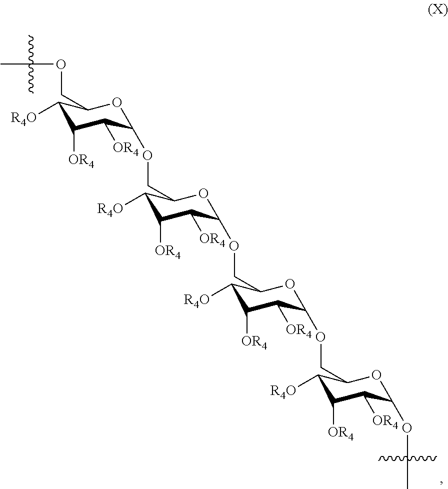

wherein R₄ is H, allyl carbamate, or mixtures thereof.

In another embodiment, the substituted dextran of formula (I-2), which is functionalized with a fluorescent label, is prepared by a method comprising the steps of:

(a) mixing a compound of formula (X):

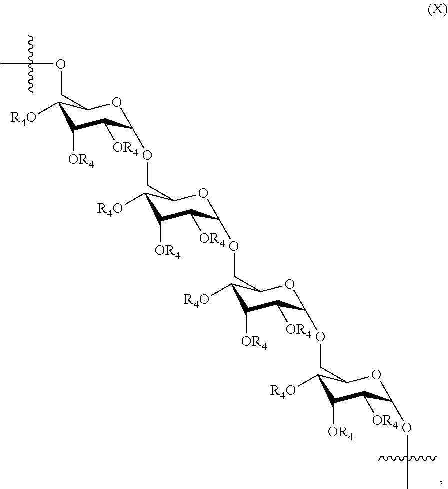

wherein R₄ is H, allyl carbamate, or mixtures thereof,

with a compound selected from the group consisting of polyethylene(glycol)diacrylate, acrylate-polyethylene(glycol)-succinimidyl valeric acid, polyethylene(glycol)acrylate, or mixtures thereof; and

(b) reacting the product of step (a) with a fluorescent label of formula (XI-a):

W₁-H (XI-a),

wherein W₁ is selected from the group consisting of:

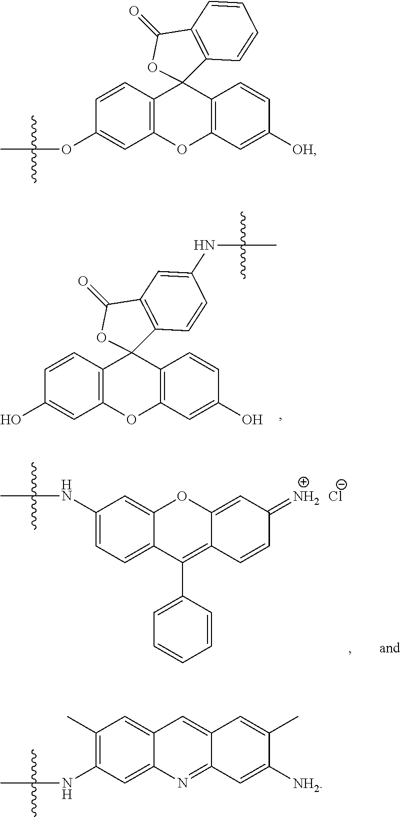 [SMILES: *Oc1ccc2c(c1)Oc1cc(O)ccc1C21OC(=O)c2ccccc21 ; *Nc1ccc2c(c1)C(=O)OC21c2ccc(O)cc2Oc2cc(O)ccc21 ; *Nc1ccc2c(-c3ccccc3)c3ccc(=[NH2+])cc-3oc2c1 ; [Cl-] ; *Nc1cc2nc3cc(N)c(C)cc3cc2cc1C]

An embodiment of the invention is a low-viscosity solution comprising (1) from about 0.5% (w/w) to about 10% (w/w) acrylate-comprising compound and/or functionalized acrylate-comprising compound selected from the group consisting of polyethylene(glycol)diacrylate, acrylate-polyethylene(glycol)-succinimidyl valeric acid, polyethylene(glycol)acrylate; (2) from about 0.5% (w/w) to about 10% (w/w) of a functionalized dextran; (3) from about 0.01% (w/w) to about 2.5% (w/w) of a UV-crosslinking catalyst and/or a visible light-crosslinking catalyst; and (4) from about 1×10² cells/μl to about 1×10⁷ cells/μl. In another embodiment, the concentration of cells is about 1×10³ cells/μl to about 1×10⁶ cells/μl. In another embodiment, the concentration of cells is about 1×10⁴ cells/μl to about 1×10⁵ cells/μl. The cells may be mammalian cells. In one embodiment, suitable cells are human cells, simian cells, porcine cells, murine cells, equine cells, bovine cells, ovine cells, feline cells, canine cells, leporine cells, or combinations thereof. In one embodiment, suitable cells are stem cells, such as mesenchymal stem cells (MSCs) (e.g., human mesenchymal stem cells (hMSCs)) which may differentiate into neurogenic, osteogenic, chondrogenic, adipogenic, tenogenic, myogenic, or stromal lineages; fibroblasts, such as primary human dermal fibroblasts (HDFs) and newborn human foreskin fibroblasts (NuFFs)); keratinocytes, such as primary human epidermal keratinocytes (HEKs); endothelial cells, such as primary endothelial cells (e.g., human umbilical vein endothelial cells (HUVECs), endothelial colony forming cells (ECFCs), primary coronary artery endothelial cells (HCAEC), primary pulmonary artery endothelial cells (HPAEC), and primary dermal microvascular endothelial cells (HDMVECs)); nerve cells; adipocytes; chondrocytes; osteocytes; myocytes; and combinations thereof. In one embodiment, this low-viscosity solution is converted to a high viscosity solution by increasing the concentration of the functionalized dextran and/or the acrylate-comprising compound and/or the functionalized acrylate-comprising compound; and/or by adding a high-viscosity, non-irritating polar solvent and/or solvent additive; and/or by partially curing the low-viscosity solution with UV-light and/or visible light.

An embodiment of the invention is a high-viscosity solution comprising (1) from about 0.5% (w/w) to about 10% (w/w) acrylate-comprising compound and/or functionalized acrylate-comprising compound selected from the group consisting of polyethylene(glycol)diacrylate, acrylate-polyethylene(glycol)-succinimidyl valeric acid, polyethylene(glycol)acrylate; (2) from about 0.5% (w/w) to about 20% (w/w) of a functionalized dextran; (3) from about 0.01% (w/w) to about 10% (w/w) of a UV-crosslinking catalyst and/or a visible light-crosslinking catalyst; and (4) from about 1×10² cells/μl to about 1×10⁷ cells/μl. In another embodiment, the concentration of cells is about 1×10³ cells/μl to about 1×10⁶ cells/μl. In another embodiment, the concentration of cells is about 1×10⁴ cells/μl to about 1×10⁵ cells/μl. The cells may be mammalian cells. In one embodiment, suitable cells are human cells, simian cells, porcine cells, murine cells, equine cells, bovine cells, ovine cells, feline cells, canine cells, leporine cells, or combinations thereof. In one embodiment, suitable cells are stem cells, such as mesenchymal stem cells (MSCs) (e.g., human mesenchymal stem cells (hMSCs)) which may differentiate into neurogenic, osteogenic, chondrogenic, adipogenic, tenogenic, myogenic, or stromal lineages; fibroblasts, such as primary human dermal fibroblasts (HDFs) and newborn human foreskin fibroblasts (NuFFs)); keratinocytes, such as primary human epidermal keratinocytes (HEKs); endothelial cells, such as primary endothelial cells (e.g., human umbilical vein endothelial cells (HUVECs), endothelial colony forming cells (ECFCs), primary coronary artery endothelial cells (HCAEC), primary pulmonary artery endothelial cells (HPAEC), and primary dermal microvascular endothelial cells (HDMVECs)); nerve cells; adipocytes; chondrocytes; osteocytes; myocytes; and combinations thereof.

In an embodiment, after the cells are mixed in the high viscosity solution the mixture is cured with UV-light, wherein the wavelength of the UV light is about 300 nm to about 400 nm. In another embodiment, after the cells are mixed in the high viscosity solution the mixture is cured with UV-light, wherein the wavelength of the UV light is about 350 nm to about 375 nm. In another embodiment, after the cells are mixed in the high viscosity solution the mixture is cured with UV-light, wherein the wavelength of the UV light is about 355 nm to about 370 nm. In another embodiment, after the cells are mixed in the high viscosity solution the mixture is cured with UV-light, wherein the wavelength of the UV light is about 365 nm.

In an embodiment, after the cells are mixed in the high viscosity solution the mixture is cured with visible light, wherein the wavelength of the visible light is about 400 to about 490 nm. In an embodiment, after the cells are mixed in the high viscosity solution the mixture is cured with visible light, wherein the wavelength of the visible light is about 405 nm to about 430 nm. In an embodiment, after the cells are mixed in the high viscosity solution the mixture is cured with visible light, wherein the wavelength of the visible light is about 410 nm to about 420 nm. In an embodiment, after the cells are mixed in the high viscosity solution the mixture is cured with visible light, wherein the wavelength of the visible light is about 415 nm.

In one embodiment, after the cells are mixed in the high viscosity solution the mixture is cured with UV light and/or visible light for about 1 min to about 100 min. In one embodiment, after the cells are mixed in the high viscosity solution the mixture is cured with UV light and/or visible light for about 5 min to about 60 min. In one embodiment, the high-viscosity solution is cured with UV light and/or visible light for about 5 min to about 50 min. In one embodiment, after the cells are mixed in the high viscosity solution the mixture is cured with UV light and/or visible light for about 5 min to about 45 min. In one embodiment, after the cells are mixed in the high viscosity solution the mixture is cured with UV light and/or visible light for about 5 min to about 40 min. In one embodiment, the high-viscosity solution is cured with UV light and/or visible light for about 5 min to about 35 min. In one embodiment, t after the cells are mixed in the high viscosity solution the mixture is cured with UV light and/or visible light for about 5 min to about 30 min. In one embodiment, after the cells are mixed in the high viscosity solution the mixture is cured with UV light and/or visible light for about 10 min to about 30 min. In one embodiment, after the cells are mixed in the high viscosity solution the mixture is cured with UV light and/or visible light for about 15 min to about 30 min. In one embodiment, the high-viscosity solution is cured with UV light and/or visible light for about 20 min to about 30 min.

Proliferation of the cells in the high viscosity solution before or after curing may be measured using a WST assay, luminescent assay, Trypan Blue assay, or another known assay in the art.

In one embodiment, the cells in the low-viscosity solution are viable for up to three days, for up to five days, for up to ten days, for up to fifteen days, or for up to thirty days. It is contemplated that the cells in the low-viscosity solution may be cryopreserved to maintain viability. It is also contemplated that cells in the low-viscosity solution may be maintained at a temperature range of about −250° C. to about 0° C. with the addition of a cryopreservation chemical, e.g., 0.1% to 20% DMSO.

In one embodiment, the cells in the high-viscosity solution are viable for up to three days, for up to five days, for up to ten days, for up to fifteen days, or for up to thirty days. It is contemplated that the cells in the high-viscosity solution may be cryopreserved to maintain viability. It is also contemplated that cells in the high-viscosity solution may be maintained at a temperature range of about −250° C. to about 0° C. with the addition of a cryopreservation chemical, e.g., 0.1% to 20% DMSO.

In one embodiment, after curing a low-viscosity solution comprising the polymerizable entities and cells and/or a high-viscosity solution comprising the polymerizable entities and cells with UV light and/or visible light, the cells in the cured EMR are viable for up to three days, for up to five days, for up to ten days, for up to fifteen days, or for up to thirty days. Without wishing to be bound to theory, it is contemplated that the solution may be stored in a freezer at below 0 celcius to maintain viability. It is also contemplated that cells in the cured EMR may be maintained in an incubator to maintain viability. The cells in the cured EMR may be maintained at a temperature range of about −50° C. to about 50° C. In one embodiment, the cells in the cured EMR may be maintained at a temperature range of about 35° C. to about 42° C. In one preferred embodiment, the cells in the cured EMR may be maintained at a temperature of about 37° C.

Without wishing to be bound by theory, it is contemplated that curing with a concentrated LED light source may reduce the curing time (e.g., about 1-10 min) compared to a conventional light source (e.g., a UV ballast, a mercury lamp, a halogen lamp).

The cell-containing CIP-EMRs of the invention also ensure patient compliance. The cell-containing CIP-EMRs of the invention provide mechanical support to the wound and increase cell migration and revascularization of the wound site. The cell-containing CIP-EMRs of the invention comprising ARBs further provide ARB release into the wound for a prolonged time period. The prolonged time period is at least 5 days, preferably at least 7 days, preferably at least 8 days, preferably at least 10 days, preferably at least 12 days, preferably at least 14 days, preferably at least 20 days. Thus, with the cell-containing CIP-EMRs of the invention, patients also do not have to clean and apply a dressing or drug to a wound daily, which leads to increased patient compliance and faster healing.

This invention provides a method of treating wounds, comprising applying an effective amount of the cell-containing CIP-EMRs of the invention to a wound in a patient in need thereof and curing the CIP-EMRs in the wound by exposing the cell-containing CIP-EMRs to UV-light and/or visible light for a suitable exposure time and intensity.

An effective amount of the cell-containing CIP-EMR of the invention is an amount that fills a wound bed, and/or conforms to wound boundaries, and/or creates a uniformly-cured EMR upon exposure to UV-light and/or visible light. Further, an effective amount of the cell-containing CIP-EMR is an amount such that wound healing occurs faster for wounds treated with the cell-containing CIP-EMR than occurs for a control, e.g., an untreated wound or a pre-cured EMR without a drug or the cells. A patient in need thereof includes e.g., a mammal having a wound as described herein. The mammal may be, e.g., a primate, e.g., a human or a monkey, a horse, a cow, a pig, a dog, a cat, or a mouse.

Suitable UV-light and/or visible light exposure times for curing the CIP-EMR in the wound, including those with or without cells, are those that create a uniformly-cured EMR and that do not inhibit the proliferation, migration, and survival of cell types that are critical to wound healing and/or that limit cytotoxicity.

Suitable UV light intensities for curing the CIP-EMR, including those with or without cells, in the wound are those that create a uniformly-cured EMR and that do not inhibit the proliferation, migration, and survival of cell types that are critical to wound healing and/or that limit cytotoxicity.

In an embodiment of this invention, exposing the CIP-EMR of the invention, including those with or without cells, to UV light is conducted using a commercially-available, handheld, long-wavelength UV lamp of adjustable intensities. Examples of suitable UV lamps are 365 nm handheld units. Other examples of suitable UV lamps are 400 W/200 WPI mercury lamps, which are capable of curing unfunctionalized EMRs in less than two minutes at a height of 6.5 inches above the material (dose=7 J/cm²), and 15 W benchtop units, which are capable of curing unfunctionalized EMRs in approximately thirty minutes at a height of 6 inches above the material (dose=10 MW/cm²). See, e.g., Sun et al., JBMA 2009. UV dosage is directly proportional to UV intensity (W/cm2) and time.

Suitable visible light intensities for curing the CIP-EMR in the wound are those that create a uniformly-cured EMR and that do not inhibit the proliferation, migration, and survival of cell types that are critical to wound healing and/or that limit cytotoxicity.

In an embodiment of this invention, exposing the CIP-EMR of the invention, including those with or without cells, to visible light is conducted using a commercially-available, handheld, visible light lamp of adjustable intensities. Visible light dosage is directly proportional to visible light intensity (W/cm2) and time. Examples of suitable visible light lamps are lamps which have a wavelength of 415 nm and/or lamps which have an output power between about 20 mW/cm² and about 55 mW/cm² and/or lamps which apply a total dose between about 10 J/cm² and about 25 J/cm².

In an embodiment of this invention, the CIP-EMRs of the invention, including those with or without cells, are cured with UV light and/or visible light at a distance of about 0.1 cm to about 100 cm from the UV light and/or visible light source, about 0.5 cm to about 50 cm from the UV light and/or visible light source, about 1 cm to about 10 cm from the UV light and/or visible light source, or about 5 cm from the UV light and/or visible light source.

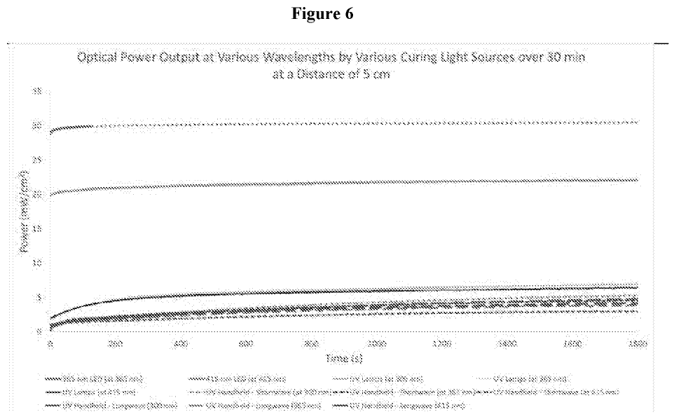

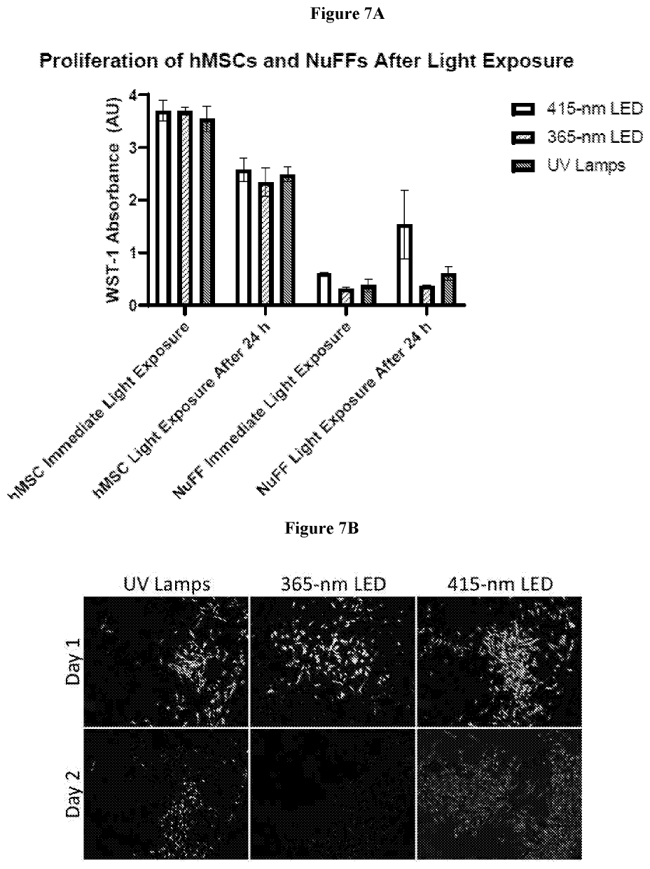

The optical power output at various wavelengths by various light sources (curing over 30 min at a distance of 5 cm) are illustrated in FIG. 6. The proliferation of exemplary cells, hMSCs and NuFFs, after having been exposed to 415 nm LED, 365 nm LED, and UV Lamps is shown in FIG. 7A and FIG. 7B. As shown in FIG. 7B, although there is noticeable cell death after 2 days in the cells exposed to a UV lamp, many cells remained viable. In the cells exposed to a 365-nm LED light, there are no obvious live cells. There was no noticeable effect on cell viability after exposure to 415-nm light.

Another embodiment of the invention is a method of treating wounds by applying an effective amount of the CIP-EMRs this invention, wherein wound healing occurs within between 1 day and 100 days after applying the CIP-EMRs to the wound. In the methods for treating wounds described herein, the CIP-EMRs of this invention include the CIP-EMRs that comprise cells.

Another embodiment of the invention is a method of treating wounds by applying an effective amount of the CIP-EMRs of this invention to the wounds, wherein wound healing occurs within between 1 day and 10 days after applying the CIP-EMRs to the wound.

Another embodiment of the invention is a method of treating wounds by applying an effective amount of the CIP-EMR of this invention to the wounds, wherein the CIP-EMR is applied twice daily, once daily, twice weekly, once weekly, twice monthly, or once monthly.

Another embodiment of the invention is a method of treating wounds by applying an effective amount of the CIP-EMR of this invention to the wounds, wherein the wounds are acute wounds or chronic wounds.

Another embodiment of the invention is a method of treating wounds by applying an effective amount of the CIP-EMR of this invention to the wounds, wherein the wounds are excision wounds or burn wounds.

Another embodiment of the invention is a method of treating wounds by applying an effective amount of the EMR-cell composition of this invention to the wounds, wherein the wounds are chronic excisional wounds, acute excisional wounds, or burn excisional wounds.

Another embodiment of the invention is a method of treating wounds by applying an effective amount of the CIP-EMR of this invention to the wounds, wherein the wounds are diabetic ulcers or pressure wounds.

In another embodiment of this invention, the CIP-EMRs of the invention are applied to surgical wounds in sharp debridement procedures. For example, as a last step in a surgery, a patient receiving operating room wound debridement may have his or her wound filled with the CIP-EMRs of the invention. These CIP-EMRs in the wound are then cured with UV light and/or visible light. The treated wound is then dressed and monitored in the hospital clinic for healing.

Another embodiment of the invention is a method of treating wounds by applying an effective amount of the CIP-EMR of this invention to the wounds, wherein the polymers of the cured EMR resulting from curing CIP-EMR with UV light and/or visible light are degraded by endogenous enzyme activity in the wound bed as healing proceeds.

This invention also provides a method of measuring the progress of wound treatment, comprising applying an effective amount of a CIP-EMR comprising a fluorescent label to a wound, curing the CIP-EMR comprising a fluorescent label with UV light and/or visible light, and measuring the amount of fluorescence in the cured EMR or in the surrounding wound via fluorescence microscopy wherein a change in the fluorescence in the wound is indicative of healing of the wound. Measuring may occur twice a day, once a day, twice a week, once a week, twice a month, or once a month.

In the methods of this invention, CIP-EMRs of this invention may be applied to the wound in the form of a pharmaceutical composition comprising the CIP-EMR and a pharmaceutically acceptable excipient.

The physical characteristics and wound healing effects of the CIP-EMRs of this invention may be assessed using any method discussed above for the interstitial EMR-drug compositions, EMR-drug conjugates, and/or EMR-fluorescent label conjugates.

The viscosity of partially-cured low-viscosity solutions of this invention is monitored over time after UV and/or visible light exposure to determine whether they continue to react after UV and/or visible light exposure. These partially-cured solutions are exposed again to UV and/or visible light to convert them from partially-cured solutions to fully-cured EMRs. The fully-cured EMRs are analyzed by testing the EMR's swelling ratio, measuring tensile strength, imaging with SEM, and calculating pore size and uniformity and cell viability and/or proliferation, and comparing the results of these analyses to control samples (e.g., unfunctionalized, cured EMRs with or without cells).

The cure rates of the CIP-EMRs of the invention, comprising or not comprising cells, are assayed in wounds of various sizes and shapes to define the relationship between wound size and curing parameters (e.g., UV and/or visible light strength and UV and/or visible light exposure time) that mimics controls (e.g., unfunctionalized EMRs). The cure rates are tested in vitro using polydimethylsiloxane (PDMS) molds of various sizes (up to 9 cm²), depths (up to 3 mm), and shapes (randomly generated). These molds are chosen to model clinically common full-thickness wound sizes.

The effect of mixing time and mixing temperatures on cure rates and outcomes of the CIP-EMRs of the invention, comprising or not comprising cells, is evaluated by varying the curing time (e.g., between 1 and 90 minutes) and/or by varying the curing temperature (e.g., between 4 C-37 C). The characteristics of the cured products resulting from the CIP-EMRs of the invention and their effects on wound healing are evaluated by, e.g., swelling ratio tests, tensile strength measurements, SEM imaging, pore-size and uniformity calculations, oxygen permeability, in wound degradation, cytotoxicity, fibroblast recruitment as described herein. Suitable unfunctionalized and/or functionalized, benchtop-cured (i.e., previously cured and not cured in place) EMRs are used as controls.

The CIP-EMRs of the invention are mechanically characterized by two methods: gelation viscosity and compressive modulus. The gelation viscosity is determined using a rheometer (e.g., a MCR 302 rheometer from Anton-Paar, Ashland, Va.) to measure changing viscosity and mechanics over UV and/or visible light exposure time. The compressive modulus, which is the destructive mechanical testing metric for quality control for unfunctionalized EMR products, is determined by unconfined parallel plate compression of the samples in a phosphate buffered saline bath using a dynamic mechanical analyzer (DMA) (e.g., a Q800 DMA from TA Instruments, New Castle, Del.). Suitable unfunctionalized and/or functionalized, benchtop-cured EMRs are used as controls.

After curing, the CIP-EMRs of the invention are tested for unreacted monomer content, aqueous swelling, DMA compression, and oscillating rheology. Suitable unfunctionalized and/or functionalized, benchtop-cured EMRs are used as controls. After curing, the products resulting from the CIP-EMRs of the invention have a shear storage modulus between about 10 Pa and about 2000 Pa, between about 10 Pa and about 1500 Pa, between about 10 Pa and about 1000 Pa, between about 10 Pa and about 500 Pa, and between about 10 Pa and about 250 Pa. Further, after curing, the products resulting from the CIP-EMRs of the invention have full oxygen permeability under normoxic conditions.

An embodiment of this invention is a method for regenerating tissue in a subject in need thereof by applying an effective amount of an EMR-cell composition of this invention and/or CIP-EMR-cell compositions of this invention to a site in tissue in need of regeneration. An effective amount of EMR-cell composition and CIP-EMR-cell compositions of this invention is an amount the increases the amount of tissue regeneration as compared to the amount of tissue regeneration obtained without treatment with the EMR-cell composition and CIP-EMR-cell compositions of the invention. In an embodiment of the invention the cells of the EMR-cell composition are stem cells that differentiate into bone linage cells, e.g., mesenchymal stem cells, or the cells are bone cells, e.g., osteoblasts, osteoclasts or osteocytes, and the tissue in need of regeneration is bone tissue. In an embodiment to the invention the cells of the EMR-cell composition are stem cells that differentiate into adipose linage cells, e.g., mesenchymal stem cells, or the cells are adipose cells, e.g., adipocyte or lipocytes, and the tissue in need of regeneration is fat tissue. In an embodiment of the invention the cells of the EMR-cell composition are stem cells that differentiate into cartilage lineage cells, e.g., mesenchymal stem cells, or the cells are cartilage cells, e.g., chondrocytes, and the tissue in need of regeneration is cartilage. The mesenchymal stem cells (MSCs) may be derived from any suitable source, e.g., the MSCs may be bone marrow-derived MSCs or adipose tissue-derived MSCs by methods known in the art. In an embodiment of the invention the MSCs may be derived from the subject to be treated, i.e., autologous MSCs, or may be derived from another subject genetically identical to the subject to be treated, i.e., syngeneic cells, or may be derived from a genetically different donor of the same species as the subject to be treated, i.e., allogeneic MSCs.

### Definitions

As used herein, the following terms have the following meanings. If not defined, a term will have its accepted meaning in the scientific and medical community.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio.

“Substituted phenyl” refers to a phenyl group which is substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio.

A “pharmaceutically acceptable excipients” is any safe, non-toxic substance that may serve as a vehicle or carrier for the EMR-drug conjugates of the invention. A pharmaceutically acceptable excipient may also be any safe, non-toxic substance that is known in the pharmaceutical industry to be useful for preparing pharmaceutical compositions, including fillers, diluents, agglutinants, binders, lubricating agents, glidants, stabilizer, colorants, wetting agents, and disintegrants. The pharmaceutically acceptable excipient may be, for example, water, saline solution, and/or polyethylene glycol.

The term “degree of substitution” is explained as follows: Each monomer unit of unfunctionalized dextran has three hydroxyl groups. If the sum of the integrated intensities of the hydroxyl peaks is 11, and the integrated intensity of the anomeric proton is 4, the ratio of the integrated intensities of the hydroxyl to the integrated intensity of the anomeric proton is 2.75. This value (2.75) is subtracted from the total number of hydroxyls (3), to calculate the degree of substitution (3−2.75=0.25). The calculated value (0.25) for the degree of substitution also corresponds to an average of one substituted hydroxyl group for every 4 monomer units.

The term “unfunctionalized EMR” represents EMRs that do not comprise a drug and/or a fluorescent label. Further, the term “unfunctionalized EMR” represents EMRs that do not comprise cells entrapped within the EMR. Suitable examples of unfunctionalized EMRs, as well as methods for preparing and characterizing unfunctionalized EMRs may be found in, e.g., U.S. Pat. Nos. 8,900,868 and 9,655,844, U.S. Pre-Grant Publication Nos. 2013/0102531 and 2015/0174154, and Sun et al., PNAS 2011, 108, 20976-20981 and Shen et al., Acellular Hydrogels for Regenerative Burn Wound Healing: Translation from a Porcine Model, Journal of Investigative Dermatology 2015.

The term “functionalized dextran” is a dextran that has at least one substituted hydroxyl group, wherein the substituent may be selected from a polymerizable compound.

The term “substituted dextran” is a dextran that has at least one substituted hydroxyl group, wherein the substituent may be, for example, a small molecule comprising a carboxylic acid, a fluorescent label, a peptide motif, and mixtures thereof. Suitable peptide motifs include peptide motifs capable of facilitating cell attachment through integrin-regulated pathways. Such peptide motifs include, for example, the arginine-glycine-aspartate tripeptide (“RGD”). In a preferred embodiment of the invention, the substituted dextran comprises an RGD substituent. In a preferred embodiment of the invention, the substituted dextran is Arg-Gly-Asp-dextran (RGD-dextran). In another preferred embodiment, the substituted dextran is Asp-Gly-Arg-dextran (DGR-dextran).

The terms “transformed” and “transforming” mean that a compound has been reacted with a suitable reagent to effect a desired chemical change. For example, a compound with an alcohol functional group may be transformed (i.e., oxidized) into a compound with a carboxylic acid functional group by reacting the compound with an alcohol functional group with a suitable oxidizing reagent. As another example, a compound with a carboxylic acid functional group may be transformed into a compound with an acyl chloride functional group by reacting the compound with a carboxylic acid functional group with a suitable chlorinating reagent (e.g., thionyl chloride). By seeing the structures of the starting compound (i.e., the compound to be transformed) and the transformed compound, a person of ordinary skill in the art would recognize the transformation and would recognize the reagents necessary to effect the transformation.

Polyethylene(glycol)diacrylate may be abbreviated as “PEGDA.” Polyethylene(glycol)diacrylate and PEGDA are used interchangeably herein.

Acrylate-polyethylene(glycol)-succinimidyl valeric acid may be abbreviated as “acrylate-PEG-SVA.” Acrylate-polyethylene(glycol)-succinimidyl valeric acid and acrylate-PEG-SVA are used interchangeably herein.

The foregoing description has been set forth merely to illustrate the invention and is not meant to be limiting. Since modifications of the described embodiments incorporating the spirit and the substance of the invention may occur to persons skilled in the art, the invention should be construed broadly to include all variations within the scope of the claims and equivalents thereof.

The contents of all references, issued patents, and published patent applications cited through this application are hereby incorporated by reference. The appropriate component, process and methods of those patents, applications and other documents may be selected for the invention and embodiments thereof.

### EXAMPLES

### Example 1—EMR Functionalization with a Fluorescent Label

Dextramate was prepared by reacting dextran (70 kDa) with allyl isocyanate molecules in DMSO to produce a dextran that is functionalized with allyl carbamate groups. Subsequently, an aqueous solution of dextramate was mixed with polyethylene(glycol)diacrylate and acrylate-polyethylene(glycol)-succinimidyl valeric acid in a polyethylene(glycol)diacrylate:acrylate-polyethylene(glycol)-succinimidyl valeric acid ratio of 80:18:2. The mixture was cured with UV light using standard curing conditions (e.g., curing at room temperature using a 15-W ballast from a height of 6 inches to deliver 15 mW/cm² over 30 minutes).

The cured product was reacted with 5-fluorescein amine. Without wishing to be bound by theory, it is contemplated that the N-hydroxysuccinimide groups of the cured product were replaced by the primary amine of 5-fluorescein amine.

### Example 2—Functionalization with an ARB

Polyethlene(glycol)acrylate (MW 5000) (0.002 mmol) was reacted with valsartan (0.02 mmol) in a ratio of 1:10 (moles polyethylene(glycol)acrylate: moles valsartan) in the presence of dicyclohexylcarbodiimide (DCC) (0.024 mol), dimethylaminopyridine (DMAP) (0.024 mmol), and dimethylformamide (DMF) (5 mL). The reaction was allowed to proceed overnight. The valsartan-functionalized polyethlene(glycol)acrylate was purified by dialysis in diH₂O for 3 days at 3500 MWCO. The purified valsartan-functionalized polyethlene(glycol)acrylate was reacted with EMR dextran PEGDA precursor mixture using cross-linking chemistry. Without wishing to be bound by theory, it is contemplated that the mechanism was photo-initiated radical cross-linking, that the initiator degraded and was covalently bound to the initiation site, and that the reaction proceeded via radical carbon-carbon bond formation until all sites were quenched.

### Example 3—Biocompatibility of the UV Curing of CIP-EMRs

To verify the biocompatibility of the UV curing of the CIP-EMRs of the invention, in vitro cell viability and proliferation are quantified under varying intensities of UV light.

A confluent layer of primary dermal fibroblasts (HDFs, ATCC) under standard cell culture conditions is exposed to increasing strengths of UV light over a range of time points. One set of primary dermal fibroblasts are exposed to UV light in the presence of the CIP-EMRs of the invention to determine if UV absorbance during curing protects the underlying cells from UV-induced cell damage. As a control, one set of primary dermal fibroblasts are exposed to UV light in the absence of the CIP-EMRs of the invention.

In vitro cell viability is quantified using a live/dead fluorescent assay (Calcein & Ethd-1, Invitrogen). The live/dead fluorescent assay detects cell lysis or death events caused by exposure to UV light.

UV exposure intensity is titrated down to identify the tolerance threshold of the cells (exposure conditions under which there is negligible adverse response). In vitro cell proliferation is quantified using a WST-1 proliferation assay (Abnova) against a control culture with no precursor or UV exposure.

### Example 4—Biocompatibility of the Visible Light Curing of CIP-EMRs

To verify the biocompatibility of the visible light curing of the CIP-EMRs of the invention, in vitro cell viability and proliferation are quantified under varying intensities of visible light.

A confluent layer of primary dermal fibroblasts (HDFs, ATCC) under standard cell culture conditions is exposed to increasing strengths of visible light over a range of time points. One set of primary dermal fibroblasts are exposed to visible light in the presence of the CIP-EMRs of the invention to determine if visible light absorbance during curing protects the underlying cells from visible light-induced cell damage. As a control, one set of primary dermal fibroblasts are exposed to visible light in the absence of the CIP-EMRs of the invention.

In vitro cell viability is quantified using a live/dead fluorescent assay (Calcein & Ethd-1, Invitrogen). The live/dead fluorescent assay detects cell lysis or death events caused by exposure to visible light.

Visible light exposure intensity is titrated down to identify the tolerance threshold of the cells (exposure conditions under which there is negligible adverse response). In vitro cell proliferation is quantified using a WST-1 proliferation assay (Abnova) against a control culture with no precursor or visible light exposure.

### Example 5—In Vitro Studies of Wound Healing with CIP-EMRs

Wound healing using the CIP-EMRs of the invention is assessed in a porcine excisional wound healing assay. Suitable unfunctionalized and/or functionalized, benchtop-cured EMRs are used as controls. In porcine excisional wound healing assays, macroscopic closure rate is quantified and the associated microscopic cellular mechanisms are determined.

Full thickness, 2.5 cm wounds are generated by circular scalpel excision after the dorsal-lateral area is shaved and sterilized. Alternatively, non-circular, irregularly shaped wounds are generated. Eight wounds (four per side) are generated between the crest of the shoulders and the coccygeal tuberosity. One side of the body (four wounds) is treated, and the other side of the body (four wounds) is the control. On one set of animals, unfunctionalized EMR is applied. On another set of animals, CIP-EMRs of the invention are applied.

To address the overall effect of the CIP-EMRs of the invention on healing, full-thickness excisional histology sections are taken at days 7, 14, 21, and at wound closure. All wounds are photographed and measured every three days during wound dressing changes.

Wounds treated with the CIP-EMRs show increased granulation tissue deposition, increased collagen organization, and complete re-epithelialization for 2.5 cm diameter, full-thickness excisional wounds as compared to an EMR control. The enhanced effects achieved by the CIP-EMRs are contemplated to be due to improved contact between the CIP-EMRs and the wound bed.

The microscopic and mechanistic performance of the CIP-EMRs of the invention is also evaluated. Histology sections, stained with Masson's Trichrome and H&E, reflect collagen deposition and remodeling, re-epithelialization, and closure rate.

Immunohistochemistry is used to stain for specific cellular markers associated with inflammatory responses and vascular reconstruction in the wound bed. Stains are also performed for macrophages (M1/M2, EMR1), neutrophils (MPO), keratinocytes, and vascular markers (CD31). qRT-PCR is also conducted on tissue removed from the center of the wound and wound edge to quantify specific markers in the wound healing arrays. Tensiometry testing (Electromatic, Cedarhurst, N.Y.) is also performed on the healed skin at closure to determine the elasticity and strength of the healed skin.

### Example 6—In Vitro Studies of Wound Healing with CIP-EMRs

Cytotoxicity of the CIP-EMRs of the invention is evaluated by culturing human fibroblasts with the CIP-EMRs of the invention in wells and then quantifying fibroblast viability, morphology, and proliferation in the presence of the CIP-EMRs of the invention.

Keratinocytes and neutrophil-like cells are cultured individually and in co-culture. CIP-EMRs of the invention are cured in several small, sterile PDMS molds with sterile-filtered precursor solution using various handheld UV lamps. A controlled number of cells are seeded onto the cell scaffold (keratinocytes) and/or into the surrounding media. Cellular infiltration and migration is characterized using time-lapse contrast microscopy. The rate of CIP-EMR degradation is characterized by measuring the mass of CIP-EMR over multiple time-points in culture.

Quantitative real-time PCR (qRT-PCR, LifeTech, Carlsbad, Calif.) is used to quantify levels of predictive wound healing markers (e.g. MMPs, TGFβ, etc.) in the cell culture and surrounding media. The ratio of the mass of the dry starting material to the mass of the freeze-dried degraded material is calculated to determine the rate of scaffold degradation over time.

### Example 7—Preparation of an EMR-Drug Conjugate Comprising Valsartan Entrapped in EMR

Dextramate (24.2 mL of a 23.1 w/w % solution), PEGDA (16.3 mL of a 8.6 w/w % solution), Irgacure (14 mL of a 0.5 w/w % solution), and valsartan (2.82 mL or 1.41 mL of a 24.8 w/w % solution or 2.82 mL of a 1:10 dilution of a 24.8 w/w % solution) are mixed together. The mixture is cured for 10 minutes. After curing, 100 mL of dH₂O is added to the cured product to create a swelled gel. Unexpectedly, valsartan does not exhibit reactivity under the curing conditions.

### Example 8—Preparation of an EMR-Cell Composition Using an Irgacure Catalyst

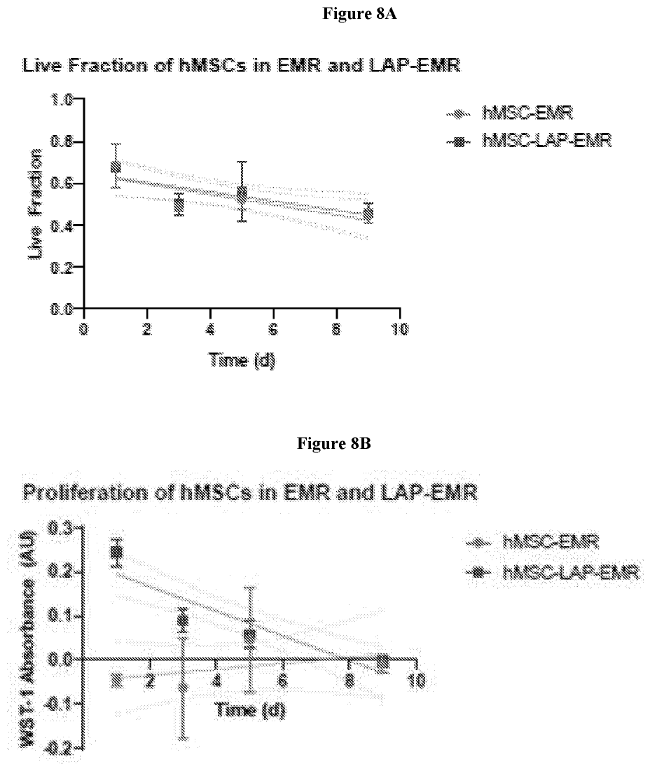

Dextramate (8.0 w/w %), PEGDA (2.0 w/w %), Irgacure (0.1% w/w % solution), and hMSCs (1,000 cells/μL in xeno-free RB Basal-MSC medium) were mixed together. The mixture (100 μl) was then poured (plated) into each well (0.32 cm²) of a 96-well plate. The plated solution was then cured for up to 30 minutes under UV light and/or visible light (about 365 nm to about 415 nm). The distance of the plated solution to the UV light and/or visible light was at least about 5 cm. After curing, 100 μL of medium (e.g., xeno-free RB Basal-MSC medium) were added to the cured EMR-cell composition. Viability of the cells was assayed immediately after curing and daily for ten days via a two-color fluorescence cell viability assay that visualizes both viable and non-viable cells. Proliferation was assayed with a WST-1 assay. The hMSCs within the cured EMR solution remained viable for up to 10 days. See FIGS. 8A and 8B.

### Example 9—Preparation of an EMR-Cell Composition Using a LAP Catalyst

Dextramate (8.0 w/w %), PEGDA (2.0 w/w %), LAP (0.1% w/w % solution), and hMSCs (1,000 cells/μL in xeno-free RB Basal-MSC medium) were mixed together. The mixture (100 μl) was then poured (plated) into each well (0.32 cm²) of a 96-well plate. The plated solution was then cured for up to 30 minutes under UV light and/or visible light (about 365 nm to about 415 nm). The distance of the plated solution to the UV light and/or visible light was at least about 5 cm. After curing, 100 μL of medium (e.g., xeno-free RB Basal-MSC medium) were added to the cured EMR-cell composition. Viability of the cells was assayed immediately after curing and daily for ten days via a two-color fluorescence cell viability assay that visualizes both viable and non-viable cells. Proliferation was assayed with a WST-1 assay. The hMSCs within the cured EMR solution remained viable for up to 10 days. See FIGS. 8A and 8B.

### Example 10—Preparation of an EMR-Cell Composition with HDFs, Using an Irgacure Catalyst

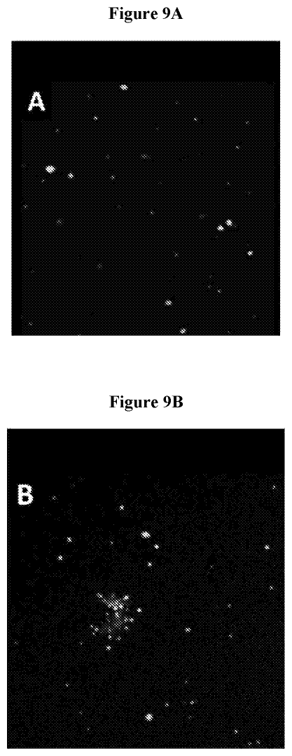

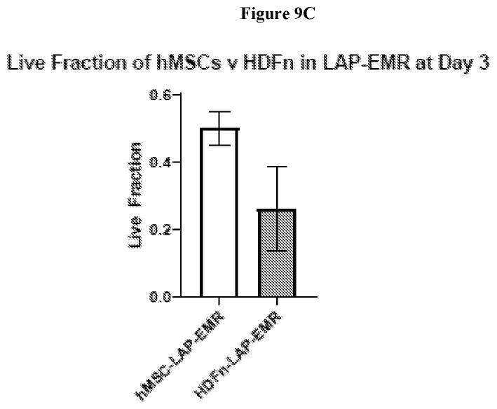

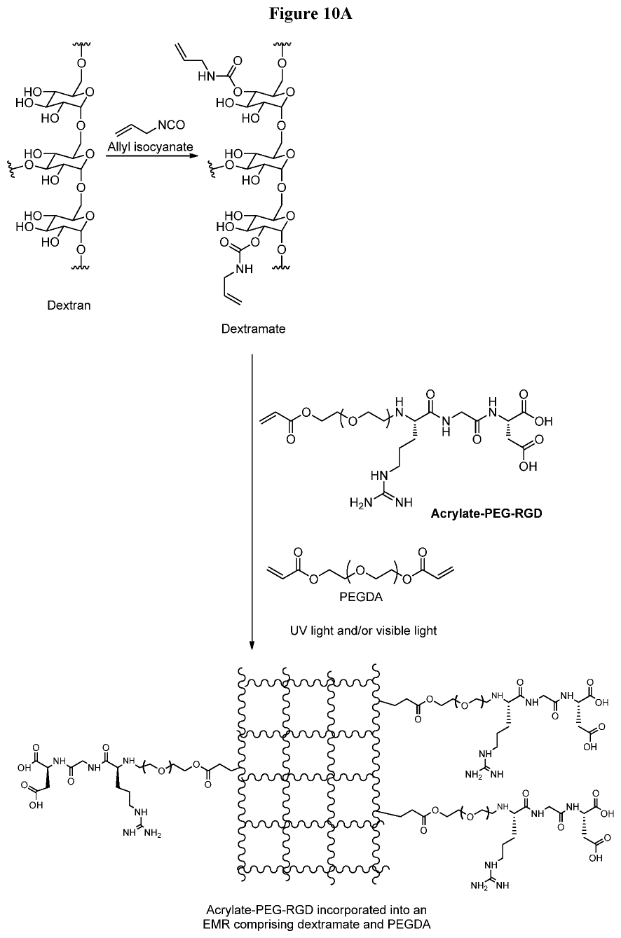

Dextramate (8.0 w/w %), PEGDA (2.0 w/w %), Irgacure (0.1% w/w % solution), and hMSCs (1,000 cells/μL in xeno-free RB Basal-MSC medium) were mixed together. The mixture (100 μl) was then poured (plated) into each well (0.32 cm²) of a 96-well plate. The plated solution was then cured for up to 30 minutes under UV light and/or visible light (about 365 nm to about 415 nm). The viability of the HDF cells was then assayed 3 days after curing and compared to the viability of hMSCs, as shown in FIG. 9A and FIG. 9C.

### Example 11—Example with Partially Cured CIP-EMR

Functionalized dextran A, B, or C, PEGDA, and LAP or Ingracure, are mixed together in a saline solution to produce a low-viscosity solution comprising functionalized dextran A, B, or C (8.0 w/w %), PEGDA (2.0 w/w %), and LAP (0.1% w/w % solution) or Irgacure (0.1% w/w % solution). The low-viscosity solution is partially cured to produce a high-viscosity solution by exposing the low-viscosity solution to UV light and/or visible light (about 365 nm to about 415 nm) for less than 30 minutes. The high-viscosity solution is then added to a wound. hMSCs (about 1,000 cells/μL to about 10,000 cells/μL in xeno-free RB Basal-MSC medium or PBS) are added to the high-viscosity solution. Thereafter, the high-viscosity solution comprising hMSCs is fully cured by exposing the partially cured solution to UV light and/or visible light (about 365 nm to about 415 nm).

Alternatively, functionalized dextran A, B, or C, PEGDA, and LAP or Ingracure, are mixed together in a saline solution to produce a low-viscosity solution comprising functionalized dextran A, B, or C (8.0 w/w %), PEGDA (2.0 w/w %), and LAP (0.1% w/w % solution) or Irgacure (0.1% w/w % solution). hMSCs (about 1,000 cells/μL to about 10,000 cells/μL in xeno-free RB Basal-MSC medium or PBS) are mixed into the low-viscosity solution. The low-viscosity solution is partially cured to produce a high-viscosity solution by exposing the low-viscosity solution to UV light and/or visible light (about 365 nm to about 415 nm). Thereafter, the high-viscosity solution comprising hMSCs is poured into a wound and then fully cured by exposing the partially cured cell containing solution to UV light and/or visible light (about 365 nm to about 415 nm).

Functionalized dextran A is functionalized with ethylamine, allyl carbamate, or mixtures thereof and has a degree of substitution between about 0.5 and about 1.

Functionalized dextran B is functionalized with ethylamine, allyl carbamate, or mixtures thereof and has a degree of substitution between about 0.25 and about 0.5.

Functionalized dextran C is functionalized with ethylamine, allyl carbamate, or mixtures thereof and has a degree of substitution between about 0.05 and about 0.25.

### Example 12—Example with Partially Cured CIP-EMR

Functionalized dextran A, B, or C, PEGDA, hMSCs, and LAP or Ingracure, are mixed together in xeno-free RB Basal-MSC medium or PBS to produce a low-viscosity solution comprising functionalized dextran A, B, or C (8.0 w/w %), PEGDA (2.0 w/w %), LAP (0.1% w/w % solution) or Irgacure (0.1% w/w % solution), and hMSCs (about 1,000 cells/μL). The low-viscosity solution is partially cured to produce a high-viscosity solution by exposing the low-viscosity solution to UV light and/or visible light (about 365 nm to about 415 nm). A sufficient amount of the high-viscosity solution is then added to a wound to fill the wound bed. Thereafter, the high-viscosity solution is fully cured by exposing the high-viscosity solution comprising hMSCs to UV light and/or visible light (about 365 nm to about 415 nm).

Functionalized dextran A is functionalized with ethylamine, allyl carbamate, or mixtures thereof and has a degree of substitution between about 0.5 and about 1.

Functionalized dextran B is functionalized with ethylamine, allyl carbamate, or mixtures thereof and has a degree of substitution between about 0.25 and about 0.5.

Functionalized dextran C is functionalized with ethylamine, allyl carbamate, or mixtures thereof and has a degree of substitution between about 0.05 and about 0.25.

### Example 13—Preparation of an EMR-Cell Composition with Acrylate-PEG-RGD or Acrylate-PEG-DGR

Functionalized dextran A, B, or C, PEGDA, Ingracure or LAP, acrylate-PEG-RGD or acrylate-PEG-DGR, and hMSCs are mixed together in xeno-free RB Basal-MSC medium or PBS to produce a mixture comprising functionalized dextran A, B, or C (8.0 w/w %), PEGDA (2.0 w/w %), Irgacure or LAP (0.1% w/w % solution), acrylate-PEG-RGD or acrylate-PEG-DGR (about 2000 Da, 10 mM), and hMSCs (1,000 cells/μL). Thereafter, the mixture is plated by distributing the mixture into the wells of any commonly used tissue culture plate, e.g., the wells (0.32 cm²) of a 96-well plate, and is then cured by exposure to UV light and/or visible light (about 365 nm to about 415 nm) for up to 30 minutes.

Functionalized dextran A is functionalized with ethylamine, allyl carbamate, or mixtures thereof and has a degree of substitution between about 0.5 and about 1.

Functionalized dextran B is functionalized with ethylamine, allyl carbamate, or mixtures thereof and has a degree of substitution between about 0.25 and about 0.5.

Functionalized dextran C is functionalized with ethylamine, allyl carbamate, or mixtures thereof and has a degree of substitution between about 0.05 and about 0.25.

### Example 14—Preparation of a CIP-EMR with Acrylate-PEG-DGR or Acrylate-PEG-RGD

Functionalized dextran A, B, or C, PEGDA, acrylate-PEG-DGR or acrylate PEG-RGD, and LAP or Ingracure are mixed together in a saline solution to produce a low-viscosity solution comprising functionalized dextran A, B, or C (8.0 w/w %), PEGDA (2.0 w/w %), LAP or Irgacure (0.1% w/w % solution), and acrylate-PEG-DGR or acrylate-PEG-RGD (about 2000 Da, 10 mM). The low-viscosity solution is partially cured to produce a high-viscosity solution by exposing the low-viscosity solution to UV light and/or visible light (about 365 nm to about 415 nm). hMSCs (about 1,000 cells/μL to about 10,000 cells/μL in xeno-free RB Basal-MSC medium or PBS) are mixed into the high-viscosity solution. A sufficient amount of the high-viscosity solution is then applied to a wound to fill the wound bed. Thereafter the high-viscosity solution is cured by exposure to UV light and/or visible light (about 365 nm to about 415 nm) for up to 30 minutes. Alternatively the high-viscosity solution is plated by distributing the high-viscosity solution into the wells of any commonly used tissue culture plate, e.g., the wells (0.32 cm²) of a 96-well plate, and is then cured by exposure to UV light and/or visible light (about 365 nm to about 415 nm) for up to 30 minutes.

Alternatively, functionalized dextran A, B, or C, PEGDA, acrylate-PEG-DGR or acrylate PEG-RGD, hMSCs, and LAP or Ingracure are mixed together in a saline solution to produce a low-viscosity solution comprising functionalized dextran A, B, or C (8.0 w/w %), PEGDA (2.0 w/w %), LAP or Irgacure (0.1% w/w % solution), hMSCs (about 1,000 cells/μL to about 10,000 cells/μL in xeno-free RB Basal-MSC medium or PBS), and acrylate-PEG-DGR or acrylate-PEG-RGD (about 2000 Da, 10 mM). The low-viscosity solution is partially cured to produce a high-viscosity solution by exposing the low-viscosity solution to UV light and/or visible light (about 365 nm to about 415 nm). A sufficient amount of the high-viscosity solution is then added to a wound to fill the wound bed. Thereafter the high-viscosity solution is cured by exposure to UV light and/or visible light (about 365 nm to about 415 nm) for up to 30 minutes. Alternatively the high-viscosity solution is plated by distributing the high-viscosity solution into the wells of any commonly used tissue culture plate, e.g., the wells (0.32 cm²) of a 96-well plate, and is then cured by exposure to UV light and/or visible light (about 365 nm to about 415 nm) for up to 30 minutes.

Functionalized dextran A may be functionalized with ethylamine, allyl carbamate, or mixtures thereof and has a degree of substitution between about 0.5 and about 1.

Functionalized dextran B may be functionalized with ethylamine, allyl carbamate, or mixtures thereof and has a degree of substitution between about 0.25 and about 0.5.

Functionalized dextran C may be functionalized with ethylamine, allyl carbamate, or mixtures thereof and has a degree of substitution between about 0.05 and about 0.25.

### Example 15—In Vivo Studies of Wound Healing

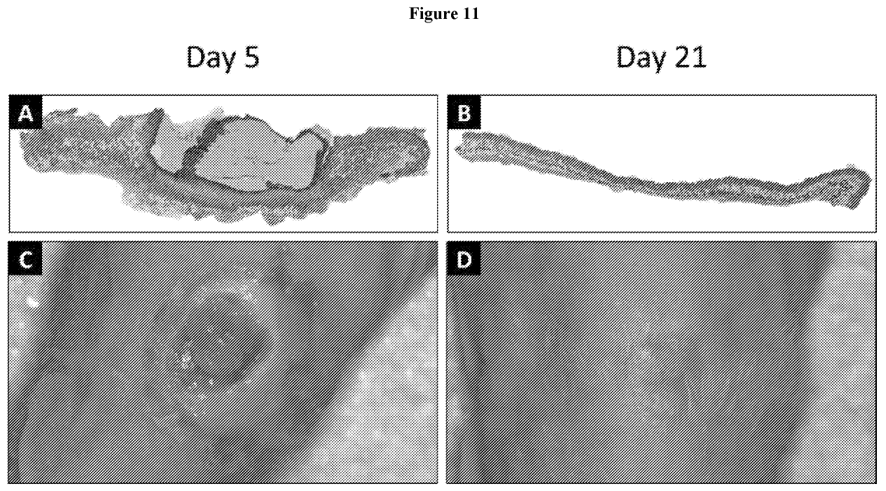

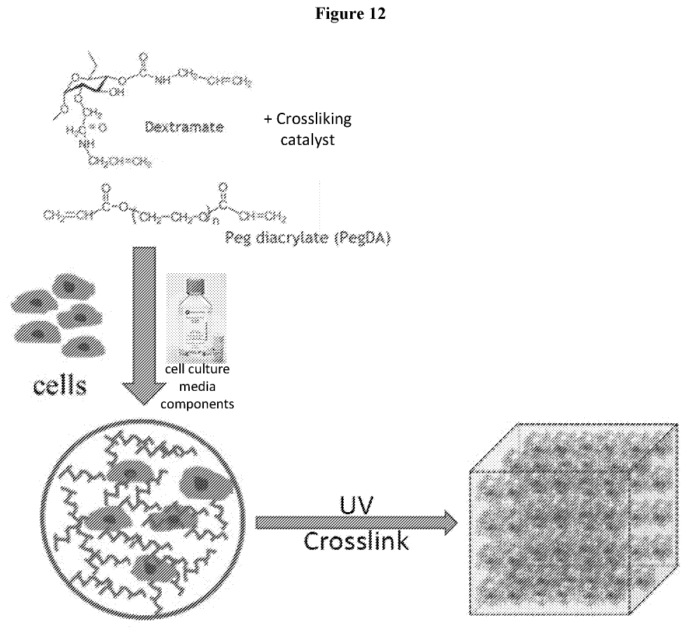

hMSCs were encapsulated in the EMR essentially as described in Example 12 above. Briefly, hMSCs from three different donors in xeno-free RB Basal-MSC medium were each mixed with a dextramate with 0.2 degree of substitution, polyethylene(glycol)diacrylate (PEGDA), and a crosslinking catalyst. The resulting mixture was poured into the wells of a 96 well plate, and then crosslinked with UV light for up to 30 minutes to form the EMR-cell compositions. The encapsulation of cells into an EMR material is illustrated in FIG. 12. The EMR-cell compositions were then implanted into third degree burns in a murine model. The implanted EMR-cell compositions contained either 100 thousand (100K) hMSCs or 1 million (1M) hMSCs. The EMR-cell compositions increased the rate of wound healing and reduced scarring compared to a control EMR (i.e., an EMR without functionalization and without encapsulated cells). Representative images from this murine study are shown in FIG. 11A-D. FIG. 11A is a histological section after 5 days of implantation. This histological section shows the EMR-cell composition filling the wound void and cell penetration. FIG. 11B is a histological section after 21 days of implantation. This histological section shows that the EMR-cell composition has degraded, and the skin, with nascent hair follicles, is restored. FIG. 11C is an image of a wound 5 days after wounding, showing the presence of the EMR-cell composition in the still-open wound. FIG. 11D is an image of a wound 21 days after wounding, showing that the EMR-cell composition has degraded and the wound has fully closed with minimal scarring.

The hMSCs in these EMR-cell compositions were viable for up to 8 days in normal culture conditions (i.e., 37° C., 5% CO₂ in an incubator) and overnight at room temperature (about 25° C.).

The hMSCs were chosen from donors with protein expression profiles most advantageous for wound healing, which can be determined by methods known in the art. See Rodgers, K. & Jadhav, S. S. Adv. Drug Deliv. Rev. (2018); Lee, D. E., Ayoub, N. & Agrawal, D. K. Stem Cell Res. Ther. (2016); King, A., Balaji, S., Keswani, S. G. & Crombleholme, T. M. Adv. Wound Care (2014); and Isackson, D., Cook, K. J., McGill, L. D. & Bachus, K. N. Med Eng Phys (2013).

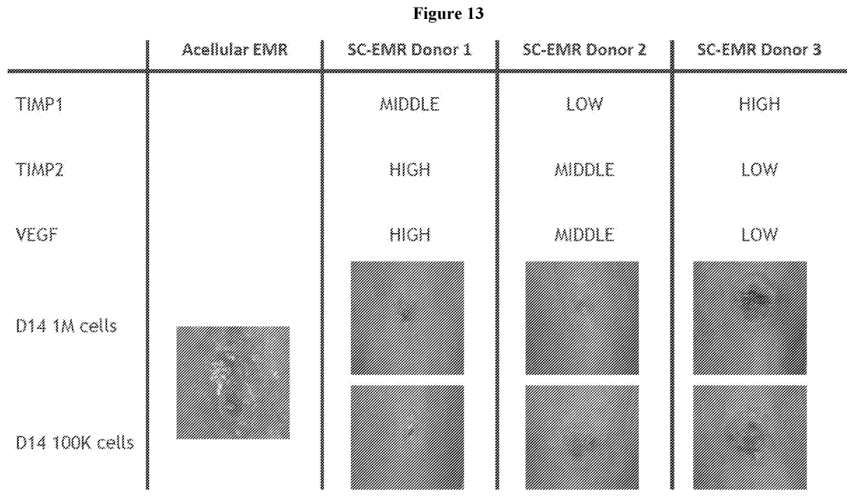

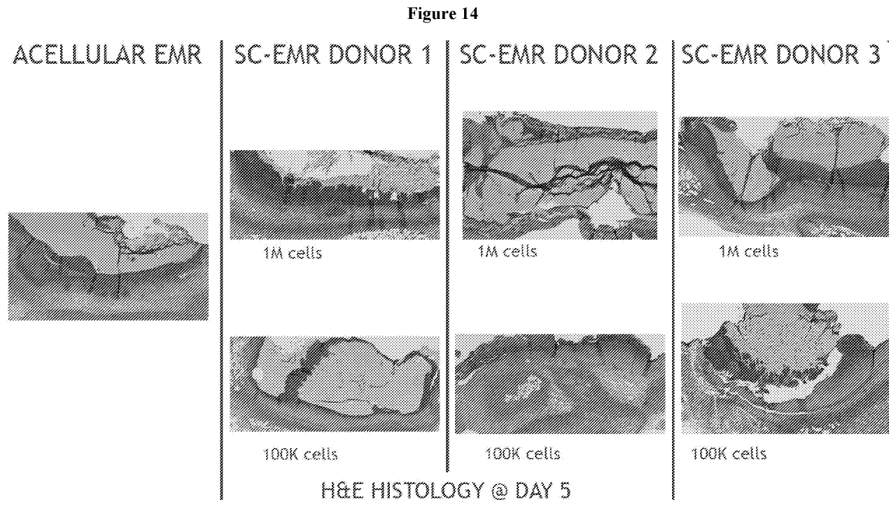

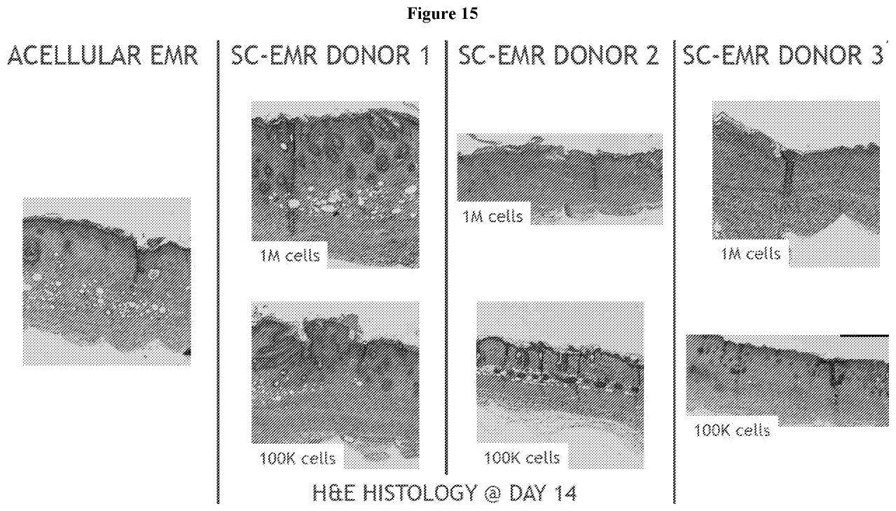

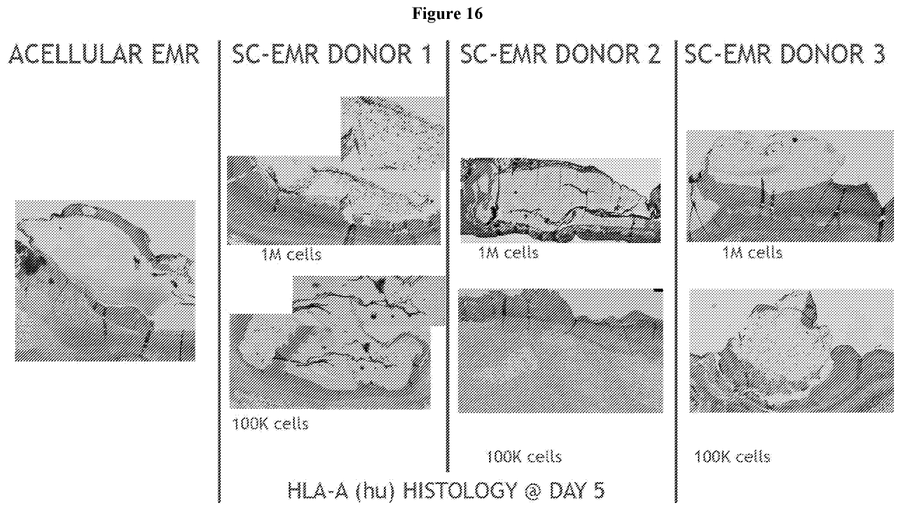

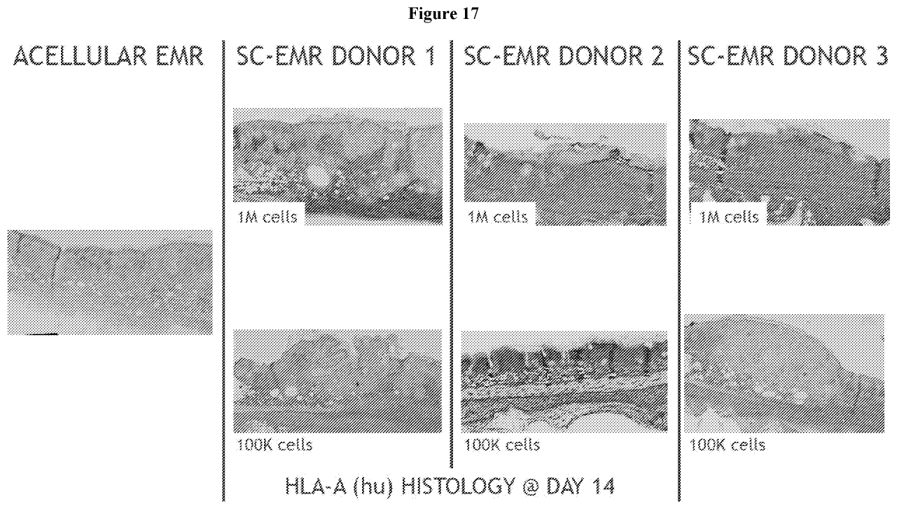

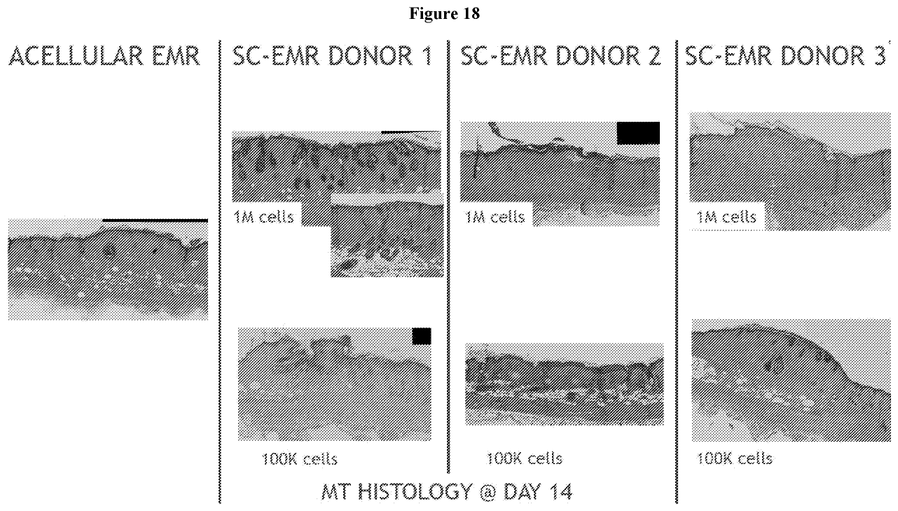

FIG. 13 shows a comparison of the of EMR-cell compositions comprising three different donor cell lines having different expression levels of TIMP1, TIMP2 and VEGF that were used in this murine study. FIG. 13 also shows a comparison of the healing of third degree burns 14 days after implanting a control EMR into the murine model and the healing of third degree burns after implanting a EMR-cell compositions comprising the three different donor cell lines at two different concentrations. FIGS. 14-18 shows different histologies of the control EMR compared to the EMR-cell compositions comprising the three different donor cell lines at two different concentrations.

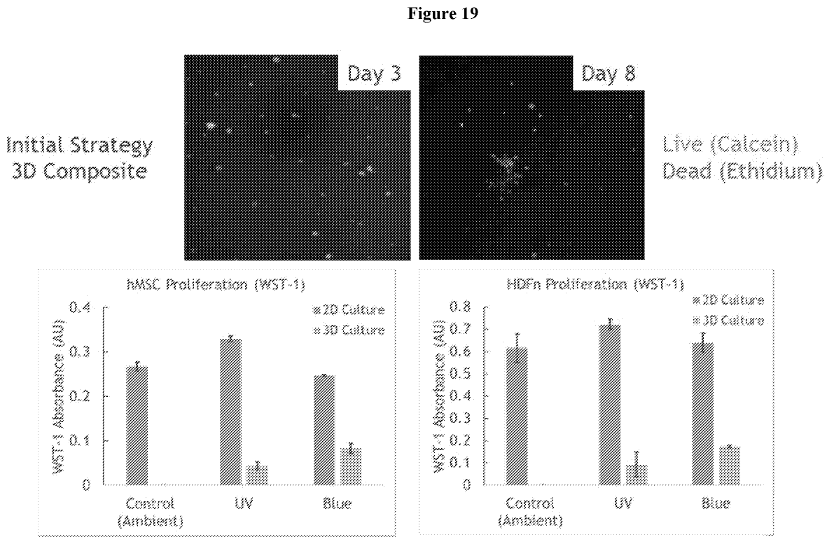

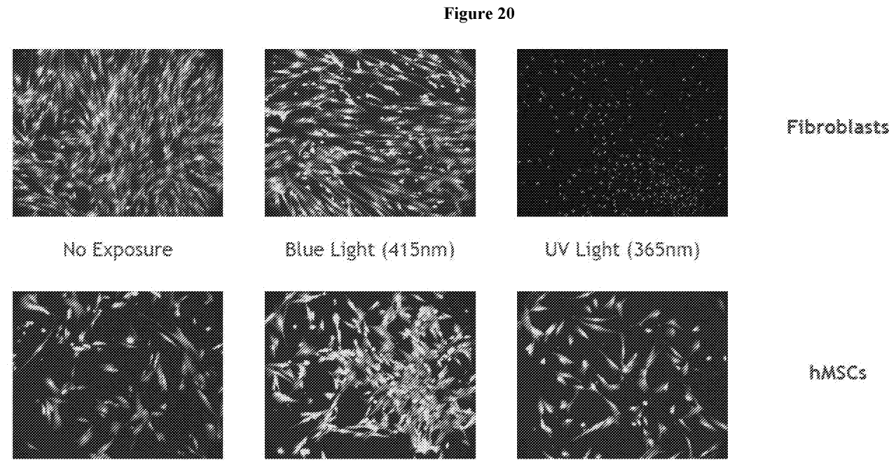

As shown in FIGS. 19 and 20, hMSCs were suitable for these studies because blue and UV light, which are needed to cure the EMR-cell composition, have no significant effect on hMSC viability. In addition, hMSCs are more resilient to light exposure than control fibroblasts.

1. A cure-in-place extracellular matrix replacement (CIP-EMR) comprising a high-viscosity solution wherein the high viscosity solution comprises:
(a) at least one functionalized dextran, wherein the functionalized dextran is a dextran functionalized with ethylamine, allylcarbamate, or mixtures thereof;
(b) cells that are stem cells; fibroblasts; keratinocytes; endothelial cells; nerve cells; adipocytes; chondrocytes; osteocytes; myocytes; or combinations thereof; and
(c) optionally comprising an acrylate-comprising compound and/or functionalized acrylate-comprising compound selected from the group consisting of polyethylene(glycol)diacrylate, polyethylene(glycol)acrylate, and acrylate-polyethylene(glycol)-succinimidyl valeric acid, Arg-Gly-Asp-polyethylene(glycol)acrylate (RGD-PEG-acrylate);
Asp-Gly-Arg-polyethylene(glycol)acrylate (DGR-PEG-acrylate); and mixtures thereof.

2. The CIP-EMR of claim 1, wherein the functionalized dextran is a dextran functionalized with allylcarbamate (functionalized dextran).

3. The CIP-EMR of claim 1, wherein the cells are stem cells which may differentiate into neurogenic, osteogenic, chondrogenic, adipogenic, tenogenic, myogenic, or stromal lineages.

4.-9. (canceled)

10. The CIP-EMR of claim 1, wherein the cells are endothelial cells.

11.-29. (canceled)

30. A method of preparing the CIP-EMR of claim 1, comprising preparing a low-viscosity solution comprising the functionalized dextran and converting the low-viscosity solution into a high-viscosity solution by increasing the concentration of the functionalized dextran and/or the acrylate-comprising compound and/or the functionalized acrylate-comprising compound; and/or by adding a high-viscosity, non-irritating polar solvent and/or solvent additive; and/or by partially curing the low-viscosity solution with UV-light and/or visible light.

31. The method of preparing the CIP-EMR of claim 30, wherein the low-viscosity solution further comprises the cells.

32. The method of preparing the CIP-EMR of claim 30, wherein the low-viscosity solution further comprises an acrylate-comprising compound and/or functionalized acrylate-comprising compound selected from the group consisting of polyethylene(glycol)diacrylate, polyethylene(glycol)acrylate, and acrylate-polyethylene(glycol)-succinimidyl valeric acid, Arg-Gly-Asp-polyethylene(glycol)acrylate (RGD-PEG-acrylate); Asp-Gly-Arg-polyethylene(glycol)acrylate (DGR-PEG-acrylate); and mixtures thereof.

33. The method of preparing the CIP-EMR of claim 30, wherein the low-viscosity solution further comprises at least one UV-crosslinking catalyst; and/or at least one visible light-crosslinking catalyst.

34-43. (canceled)

44. The method of preparing the CIP-EMR of claim 30, further comprising adding cells to the high-viscosity solution, wherein the cells are selected from the group consisting of stem cells; fibroblasts; keratinocytes; endothelial cells; nerve cells; adipocytes; chondrocytes; osteocytes; myocytes; and combinations thereof.

45. The method of preparing the CIP-EMR of claim 44, wherein the stem cells are stem cells which may differentiate into neurogenic, osteogenic, chondrogenic, adipogenic, tenogenic, myogenic, or stromal lineages.

46.-54. (canceled)

55. A method of treating wounds, comprising applying an effective amount of the CIP-EMR of claim 1 to a wound in a subject in need thereof and curing the CIP-EMRs by exposing the CIP-EMR in the wound to UV-light and/or visible light for a suitable exposure time and a suitable UV light intensity and/or visible light intensity to cure the CIP-EMR in the wound.

56. The method of treating wounds of claim 55, wherein the CIP-EMR is applied to and cured in the wound twice daily, once daily, twice weekly, once weekly, twice monthly, or once monthly.

57.-62. (canceled)

63. A composition comprising an extracellular matrix replacement and cells (an EMR-cell composition), wherein the EMR has been prepared by
(a) mixing
(i) at least one functionalized dextran, wherein the functionalized dextran is a dextran functionalized with ethylamine, allylcarbamate, or mixtures thereof;
(ii) cells, wherein the cells are selected from the group consisting of stem cells; fibroblasts; keratinocytes; endothelial cells; nerve cells; adipocytes; chondrocytes; osteocytes; myocytes; and combinations thereof;
(iii) optionally comprising an acrylate-comprising compound and/or functionalized acrylate-comprising compound selected from the group consisting of polyethylene(glycol)diacrylate, polyethylene(glycol)acrylate, and acrylate-polyethylene(glycol)-succinimidyl valeric acid, Arg-Gly-Asp-polyethylene(glycol)acrylate (RGD-PEG-acrylate); Asp-Gly-Arg-polyethylene(glycol)acrylate (DGR-PEG-acrylate); and mixtures thereof; and
(iv) optionally comprising at least one UV-crosslinking catalyst and/or at least one visible light-crosslinking catalyst; and
(b) curing the mixture obtained in step (a) with UV light and/or visible light.

64. The EMR-cell composition of claim 63, wherein the functionalized dextran is a dextran functionalized with allylcarbamate (functionalized dextran).

65. The EMR-cell composition of claim 63, wherein the stem cells are stem cells which may differentiate into neurogenic, osteogenic, chondrogenic, adipogenic, tenogenic, myogenic, or stromal lineages.

66.-79. (canceled)

80. A method of treating wounds, comprising applying an effective amount of the EMR-cell composition of claim 63 to a wound in a subject in need thereof.

81. The method of treating wounds of claim 80, wherein the EMR-cell composition is applied to the wound twice daily, once daily, twice weekly, once weekly, twice monthly, or once monthly.

82. The method of treating wounds of claim 80, wherein the wounds are acute wounds, chronic wounds, excision wounds, burn wounds, diabetic ulcers, or pressure wounds.

83. (canceled)

84. (canceled)

85. A method for regenerating tissue, comprising applying an effective amount of the CIP-EMR of claim 1, to a tissue in need of regeneration and curing CIP-EMRs in the tissue by exposing the CIP-EMRs in the tissue to UV-light and/or visible light for a suitable exposure time and a suitable UV light intensity and/or visible light intensity.

86.-91. (canceled)

92. A method for regenerating tissue, comprising applying an effective amount of the EMR-cell composition of claim 63 to a tissue in need of regeneration and curing EMRs in the tissue by exposing the EMRs in the tissue to UV-light and/or visible light for a suitable exposure time and a suitable UV light intensity and/or visible light intensity.
